SEQUENCE LISTING (68 sequences)

SEQ ID NO: 1 - protein, 345 aa, synthetic construct
MVQTSLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 2 - DNA, 1038 bp, synthetic construct
  source 1..1038: mol_type = other DNA
atggtccaaactagtttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 3 - protein, 423 aa, synthetic construct
MPQFGILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 4 - DNA, 1272 bp, synthetic construct
  source 1..1272: mol_type = other DNA
ttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgaaagaaatgaggtcatcaaatgtcttccaatgtgagattttgggccattttttatagcaaagattgaataaggcgcatttttcttcaaagctttattgtacgatctgactaagttatcttttaataattggtattcctgtttattgcttgaagaattgccggtcctatttactcgttttaggactggttcagaattcctcaaaaattcatccaaatatacaagtggatcgatcctaccccttgcgctaaagaagtatatgtgcctactaacgcttgtctttgtctctgtcactaaacactggattattactcccagatacttattttggactaatttaaatgatttcggatcaacgttcttaatatcgctgaatcttccacaattgatgaaagtagctaggaagaggaattggtataaagtttttgtttttgtaaatctcgaagtatactcaaacgaatttagtattttctcagtgatctcccagatgctttcaccctcacttagaagtgctttaagcatttttttactgtggctatttcccttatctgcttcttccgatgattcgaactgtaattgcaaactacttacaatatcagtgatatcagattgatgtttttgtccatagtaaggaataattgtaaattcccaagcaggaatcaatttctttaatgaggcttccagaattgttgctttttgcgtcttgtatttaaactggagtgatttattgacaatatcgaaactcagcgaattgcttatgatagtattatagctcatgaatgtggctctcttgattgctgttccgttatgtgtaatcatccaacataaataggttagttcagcagcacataatgctattttctcacctgaaggtctttcaaacctttccacaaactgacgaacaagcaccttaggtggtgttttacataatataccaaattgtggcat

SEQ ID NO: 5 - DNA, 6286 bp, synthetic construct
  source 1..6286: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaattcaaacactagtgaagttcctattcttcaaatagtataggaacttcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtccggcctatacactcacagtggtttggcatatatttggtgaaattttttaaggaaaaattagtgttggtttcgatatatggtagctttttctctaacataatttgaataattcagcaaagccctactaccagctgtacttctgcagcctcttccattctttccagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtaggaaggtttaaacttcctgcagcccgggctgggcataaaagtcagggcagagccatctattgcttacatttgcttctcttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggctagcgccaccatgtctagtgatgatgaggctactgctgactctcaacattctactcctccaaaaaagaagagaaaggtagaagaccccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggaggccgctacctggcggacttcaagaccacctacaaggccaagaagcccgtgcagatgcccggcgcctacaacgtggaccgcaagttggacatcacctcccacaacgaggactacaccgtggtggaacagtacgaacgctccgagggccgccactccaccggcggcatggacgagctgtataagtaagwwsswwswwsswsswvhdbcctgcgttgttgatattgtggaccaattattcgtatagcatacattatacgaagttatgtagacaatcctttggtccgaagtatgtacaacatttgcggcctaaagacaaaccgctccatggtgaaaacgactaagggtacccaggagaatatgagctataaattgctataatgtatgctatacgaagttatctagagcgttgtaccctattcagaggttacacgaccgaattgggattcaatcgttcgaagttcctattcttcaaatagtataggaacttcaccggtgcgcgtcctggattcgcgttcgcgcgtacatccagctgacgagtcccaaataggacgaaacgcgcgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcagcgctagtgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 34 bp, unidentified
  source 1..34: mol_type = unassigned DNA
gaagttcctattctctagaaagtataggaacttc

SEQ ID NO: 7 - DNA, 34 bp, synthetic construct
  source 1..34: mol_type = other DNA
gaagttcctattctctagatagtataggaacttc

SEQ ID NO: 8 - DNA, 34 bp, synthetic construct
  source 1..34: mol_type = other DNA
gaagttcctattctctacttagtataggaacttc

SEQ ID NO: 9 - DNA, 34 bp, synthetic construct
  source 1..34: mol_type = other DNA
gaagttcctattcttcaaatagtataggaacttc

SEQ ID NO: 10 - DNA, 34 bp, synthetic construct
  source 1..34: mol_type = other DNA
gaagttcctattctctagaaggtataggaacttc

SEQ ID NO: 11 - DNA, 34 bp, synthetic construct
  source 1..34: mol_type = other DNA
gaagttcctattcttcaaaaggtataggaacttc

SEQ ID NO: 12 - DNA, 34 bp, synthetic construct
  source 1..34: mol_type = other DNA
gaagttcctattctcatataagtataggaacttc

SEQ ID NO: 13 - DNA, 34 bp, synthetic construct
  source 1..34: mol_type = other DNA
gaagttcctattctatcagaagtataggaacttc

SEQ ID NO: 14 - DNA, 34 bp, synthetic construct
  source 1..34: mol_type = other DNA
gaagttcctattctctaaaaagtataggaacttc

SEQ ID NO: 15 - DNA, 34 bp, synthetic construct
  source 1..34: mol_type = other DNA
gaagttcctatagtttctagactataggaacttc

SEQ ID NO: 16 - DNA, 34 bp, synthetic construct
  source 1..34: mol_type = other DNA
gaagtttctatagtttctagactatagaaacttc

SEQ ID NO: 17 - DNA, 34 bp, synthetic construct
  source 1..34: mol_type = other DNA
gaagtttctattctctagaaagtatagaaacttc

SEQ ID NO: 18 - DNA, 34 bp, unidentified
  source 1..34: mol_type = unassigned DNA
  misc_difference 14: note = a, c, t, g, unknown or other
  misc_difference 15: note = a, c, t, g, unknown or other
  misc_difference 16: note = a, c, t, g, unknown or other
  misc_difference 19: note = a, c, t, g, unknown or other
  misc_difference 20: note = a, c, t, g, unknown or other
  misc_difference 21: note = a, c, t, g, unknown or other
ataacttcgtatannntannntatacgaagttat

SEQ ID NO: 19 - DNA, 34 bp, unidentified
  source 1..34: mol_type = unassigned DNA
ataacttcgtatagcatacattatacgaagttat

SEQ ID NO: 20 - DNA, 34 bp, unidentified
  source 1..34: mol_type = unassigned DNA
taccgttcgtatagcatacattatacgaagttat

SEQ ID NO: 21 - DNA, 34 bp, unidentified
  source 1..34: mol_type = unassigned DNA
ataacttcgtatagcatacattatacgaacggta

SEQ ID NO: 22 - DNA, 34 bp, unidentified
  source 1..34: mol_type = unassigned DNA
aattattcgtatagcatacattatacgaagttat

SEQ ID NO: 23 - DNA, 34 bp, unidentified
  source 1..34: mol_type = unassigned DNA
ataacttcgtatagcatacattatacgaataatt

SEQ ID NO: 24 - DNA, 34 bp, unidentified
  source 1..34: mol_type = unassigned DNA
taacgttcgtatagcatacattatacgaagttat

SEQ ID NO: 25 - DNA, 34 bp, unidentified
  source 1..34: mol_type = unassigned DNA
ataacttcgtatagcatacattatacgaacgtta

SEQ ID NO: 26 - DNA, 34 bp, unidentified
  source 1..34: mol_type = unassigned DNA
ataaattgctatagcatacattatacgaagttat

SEQ ID NO: 27 - DNA, 34 bp, unidentified
  source 1..34: mol_type = unassigned DNA
ataacttcgtatagcatacattatagcaatttat

SEQ ID NO: 28 - DNA, 13 bp, unidentified
  source 1..13: mol_type = unassigned DNA
ataacttcgtata

SEQ ID NO: 29 - DNA, 13 bp, unidentified
  source 1..13: mol_type = unassigned DNA
tatacgaagttat

SEQ ID NO: 30 - DNA, 13 bp, unidentified
  source 1..13: mol_type = unassigned DNA
taccgttcgtata

SEQ ID NO: 31 - DNA, 13 bp, unidentified
  source 1..13: mol_type = unassigned DNA
tatacgaacggta

SEQ ID NO: 32 - DNA, 69 bp, synthetic construct
  source 1..69: mol_type = other DNA
gcgcgtcctggattccactgcttcggcaggtacatccagctgacgagtcccaaataggacgaaacgcgc

SEQ ID NO: 33 - DNA, 65 bp, synthetic construct
  source 1..65: mol_type = other DNA
gcgcgtcctggattccactttcgaggtacatccagctgacgagtcccaaataggacgaaacgcgc

SEQ ID NO: 34 - DNA, 63 bp, synthetic construct
  source 1..63: mol_type = other DNA
gcgcgtcctggattccacttcgggtacatccagctgacgagtcccaaataggacgaaacgcgc

SEQ ID NO: 35 - DNA, 61 bp, synthetic construct
  source 1..61: mol_type = other DNA
gcgcgtcctggattccattcggtacatccagctgacgagtcccaaataggacgaaacgcgc

SEQ ID NO: 36 - DNA, 63 bp, synthetic construct
  source 1..63: mol_type = other DNA
gcgcgtcctggattcgcattcgcgtacatccagctgacgagtcccaaataggacgaaacgcgc

SEQ ID NO: 37 - DNA, 65 bp, synthetic construct
  source 1..65: mol_type = other DNA
gcgcgtcctggattcgcgattccgcgtacatccagctgacgagtcccaaataggacgaaacgcgc

SEQ ID NO: 38 - DNA, 67 bp, synthetic construct
  source 1..67: mol_type = other DNA
gcgcgtcctggattcgcgcattcgcgcgtacatccagctgacgagtcccaaataggacgaaacgcgc

SEQ ID NO: 39 - DNA, 65 bp, synthetic construct
  source 1..65: mol_type = other DNA
gcgcgtcctggattcgcgttcgcgcgtacatccagctgacgagtcccaaataggacgaaacgcgc

SEQ ID NO: 40 - DNA, 65 bp, synthetic construct
  source 1..65: mol_type = other DNA
gcgcgtcctggattcgcggaaacgcgtacatccagctgacgagtcccaaataggacgaaacgcgc

SEQ ID NO: 41 - DNA, 65 bp, synthetic construct
  source 1..65: mol_type = other DNA
gcgcgtcctggattcgcgtcaccgcgtacatccagctgacgagtcccaaataggacgaaacgcgc

SEQ ID NO: 42 - DNA, 70 bp, synthetic construct
  source 1..70: mol_type = other DNA
gcgcgtcctggattcgcgagaggaggccgcgtacatccagctgacgagtcccaaataggacgaaacgcgc

SEQ ID NO: 43 - DNA, 72 bp, synthetic construct
  source 1..72: mol_type = other DNA
gcgcgtcctggattcggccagaggaggcggccgtacatccagctgacgagtcccaaataggacgaaacgcgc

SEQ ID NO: 44 - DNA, 20 bp, synthetic construct
  source 1..20: mol_type = other DNA
tttttttttttttttttttt

SEQ ID NO: 45 - DNA, 22 bp, synthetic construct
  source 1..22: mol_type = other DNA
  misc_difference 21: note = a, c, t, g, unknown or other
ttttttttttttttttttttnv

SEQ ID NO: 46 - DNA, 59 bp, synthetic construct
  source 1..59: mol_type = other DNA
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
ctttccctacacgacgctcttccgatctnnnnnnnnggcatggacgagctgtataagta

SEQ ID NO: 47 - DNA, 49 bp, synthetic construct
  source 1..49: mol_type = other DNA
ggagttcagacgtgtgctcttccgatctaagcagcgtatccacatagcg

SEQ ID NO: 48 - DNA, 58 bp, synthetic construct
  source 1..58: mol_type = other DNA
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
ctttccctacacgacgctcttccgatctnnnnnnnggcatggacgagctgtataagta

SEQ ID NO: 49 - DNA, 50 bp, synthetic construct
  source 1..50: mol_type = other DNA
  misc_difference 29: note = a, c, t, g, unknown or other
ggagttcagacgtgtgctcttccgatctnaagcagcgtatccacatagcg

SEQ ID NO: 50 - DNA, 57 bp, synthetic construct
  source 1..57: mol_type = other DNA
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
ctttccctacacgacgctcttccgatctnnnnnnggcatggacgagctgtataagta

SEQ ID NO: 51 - DNA, 51 bp, synthetic construct
  source 1..51: mol_type = other DNA
  misc_difference 29: note = a, c, t, g, unknown or other
  misc_difference 30: note = a, c, t, g, unknown or other
ggagttcagacgtgtgctcttccgatctnnaagcagcgtatccacatagcg

SEQ ID NO: 52 - DNA, 56 bp, synthetic construct
  source 1..56: mol_type = other DNA
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
ctttccctacacgacgctcttccgatctnnnnnggcatggacgagctgtataagta

SEQ ID NO: 53 - DNA, 52 bp, synthetic construct
  source 1..52: mol_type = other DNA
  misc_difference 29: note = a, c, t, g, unknown or other
  misc_difference 30: note = a, c, t, g, unknown or other
  misc_difference 31: note = a, c, t, g, unknown or other
ggagttcagacgtgtgctcttccgatctnnnaagcagcgtatccacatagcg

SEQ ID NO: 54 - DNA, 55 bp, synthetic construct
  source 1..55: mol_type = other DNA
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
ctttccctacacgacgctcttccgatctnnnnggcatggacgagctgtataagta

SEQ ID NO: 55 - DNA, 53 bp, synthetic construct
  source 1..53: mol_type = other DNA
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
ggagttcagacgtgtgctcttccgatctnnnnaagcagcgtatccacatagcg

SEQ ID NO: 56 - DNA, 54 bp, synthetic construct
  source 1..54: mol_type = other DNA
  misc_difference 29: note = a, c, t, g, unknown or other
  misc_difference 30: note = a, c, t, g, unknown or other
  misc_difference 31: note = a, c, t, g, unknown or other
ctttccctacacgacgctcttccgatctnnnggcatggacgagctgtataagta

SEQ ID NO: 57 - DNA, 54 bp, synthetic construct
  source 1..54: mol_type = other DNA
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
ggagttcagacgtgtgctcttccgatctnnnnnaagcagcgtatccacatagcg

SEQ ID NO: 58 - DNA, 53 bp, synthetic construct
  source 1..53: mol_type = other DNA
  misc_difference 29: note = a, c, t, g, unknown or other
  misc_difference 30: note = a, c, t, g, unknown or other
ctttccctacacgacgctcttccgatctnnggcatggacgagctgtataagta

SEQ ID NO: 59 - DNA, 55 bp, synthetic construct
  source 1..55: mol_type = other DNA
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
ggagttcagacgtgtgctcttccgatctnnnnnnaagcagcgtatccacatagcg

SEQ ID NO: 60 - DNA, 52 bp, synthetic construct
  source 1..52: mol_type = other DNA
  misc_difference 29: note = a, c, t, g, unknown or other
ctttccctacacgacgctcttccgatctnggcatggacgagctgtataagta

SEQ ID NO: 61 - DNA, 56 bp, synthetic construct
  source 1..56: mol_type = other DNA
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
ggagttcagacgtgtgctcttccgatctnnnnnnnaagcagcgtatccacatagcg

SEQ ID NO: 62 - DNA, 436 bp, synthetic construct
  source 1..436: mol_type = other DNA
  misc_difference 66: note = a, c, t, g, unknown or other
  misc_difference 67: note = a, c, t, g, unknown or other
  misc_difference 68: note = a, c, t, g, unknown or other
  misc_difference 69: note = a, c, t, g, unknown or other
  misc_difference 70: note = a, c, t, g, unknown or other
  misc_difference 71: note = a, c, t, g, unknown or other
  misc_difference 72: note = a, c, t, g, unknown or other
  misc_difference 73: note = a, c, t, g, unknown or other
  misc_difference 74: note = a, c, t, g, unknown or other
  misc_difference 75: note = a, c, t, g, unknown or other
  misc_difference 76: note = a, c, t, g, unknown or other
  misc_difference 77: note = a, c, t, g, unknown or other
  misc_difference 78: note = a, c, t, g, unknown or other
  misc_difference 79: note = a, c, t, g, unknown or other
  misc_difference 80: note = a, c, t, g, unknown or other
  misc_difference 81: note = a, c, t, g, unknown or other
  misc_difference 82: note = a, c, t, g, unknown or other
  misc_difference 83: note = a, c, t, g, unknown or other
  misc_difference 84: note = a, c, t, g, unknown or other
agtacgaacgctccgagggccgccactccaccggcggcatggacgagctgtataagtaagatatcnnnnnnnnnnnnnnnnnnnaagcttctgcgttgttgatattgtggacctcggaattcaattattcgtatagcatacattatacgaagttatgtagacaatcctttggtccgaagtatgtacaacatttgcggcctaaagacaaaccgctccatggtgaaaacgactaagggtacccaggagaatatgagctataaattgctataatgtatgctatacgaagttatgaattcatcgataatcaacctctggattacaaaatttgtgaaagattgactggtattcttaactatgttgctccttttacgctatgtggatacgctgctttaatgcctttgtatcatgctattgcttcccgtatggctttcatttt

SEQ ID NO: 63 - DNA, 436 bp, synthetic construct
  source 1..436: mol_type = other DNA
  misc_difference 66: note = a, c, t, g, unknown or other
  misc_difference 67: note = a, c, t, g, unknown or other
  misc_difference 68: note = a, c, t, g, unknown or other
  misc_difference 69: note = a, c, t, g, unknown or other
  misc_difference 70: note = a, c, t, g, unknown or other
  misc_difference 71: note = a, c, t, g, unknown or other
  misc_difference 72: note = a, c, t, g, unknown or other
  misc_difference 73: note = a, c, t, g, unknown or other
  misc_difference 74: note = a, c, t, g, unknown or other
  misc_difference 75: note = a, c, t, g, unknown or other
  misc_difference 76: note = a, c, t, g, unknown or other
  misc_difference 77: note = a, c, t, g, unknown or other
  misc_difference 78: note = a, c, t, g, unknown or other
  misc_difference 79: note = a, c, t, g, unknown or other
  misc_difference 80: note = a, c, t, g, unknown or other
  misc_difference 81: note = a, c, t, g, unknown or other
  misc_difference 82: note = a, c, t, g, unknown or other
  misc_difference 83: note = a, c, t, g, unknown or other
  misc_difference 84: note = a, c, t, g, unknown or other
agtacgaacgctccgagggccgccactccaccggcggcatggacgagctgtataagtaagatatcnnnnnnnnnnnnnnnnnnnaagcttctgcgttgttgatattgtggacctcggaattcaattattcgtatagcatacattatagcaatttatagctcatattctcctgggtacccttagtcgttttcaccatggagcggtttgtctttaggccgcaaatgttgtacatacttcggaccaaaggattgtctacataacttcgtataatgtatgctatacgaagttatgaattcatcgataatcaacctctggattacaaaatttgtgaaagattgactggtattcttaactatgttgctccttttacgctatgtggatacgctgctttaatgcctttgtatcatgctattgcttcccgtatggctttcatttt

SEQ ID NO: 64 - DNA, 19 bp, synthetic construct
  source 1..19: mol_type = other DNA
sswswwwsswswwswvhdb

SEQ ID NO: 65 - DNA, 19 bp, synthetic construct
  source 1..19: mol_type = other DNA
wsswssswswsswwsvhdb

SEQ ID NO: 66 - DNA, 19 bp, synthetic construct
  source 1..19: mol_type = other DNA
swswsswwsswwswsvhdb

SEQ ID NO: 67 - protein, 258 aa, synthetic construct
MSSDDEATADSQHSTPPKKKRKVEDPMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 68 - protein, 286 aa, synthetic construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

(19) United States
(12) Patent Application Publication
(10) Pub. No.: US 2025/0034556 A1
(43) Pub. Date: Jan. 30, 2025

(54) COMPOSITIONS AND METHODS FOR SCREENING CIS REGULATORY ELEMENTS

(71) Applicant: The Broad Institute, Inc., Cambridge, MA (US)

(72) Inventors: Binhui Zhao, Cambridge, MA (US); Benjamin E. Deverman, Cambridge, MA (US)

(21) Appl. No.: 18/912,282

(22) Filed: Oct. 10, 2024

Related U.S. Application Data
(63) Continuation of application No. PCT/US2023/018291, filed on Apr. 12, 2023.
(60) Provisional application No. 63/330,078, filed on Apr. 12, 2022.

(51) Int. Cl. C12N 15/10; C12N 15/86; C12Q 1/6869

(52) U.S. Cl. CPC C12N 15/1093; C12N 15/86; C12Q 1/6869; C12N 2750/14143; C12N 2800/30; C12N 2830/48; C12N 2830/50; C12N 2830/52

(57) ABSTRACT

The invention provides compositions and methods that are useful for screening gene regulatory elements for cell type-specific expression in vivo.

### CROSS-REFERENCE TO RELATED APPLICATIONS

This application is a continuation of International Application No. PCT/US2023/018291 filed Apr. 12, 2023, which claims priority to and the benefit of U.S. Provisional Application No. 63/330,078, filed Apr. 12, 2022, the disclosures of which are incorporated herein by reference in their entireties.

### STATEMENT OF RIGHTS TO INVENTIONS MADE UNDER FEDERALLY SPONSORED RESEARCH

This invention was made with government support under Grant No. MH120096 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on May 23, 2023, is named “167741-024202US_SequenceListing.xml” and is 113 kilobytes in size.

### BACKGROUND OF THE INVENTION

Functional characterization of cis regulatory elements (e.g., enhancers) is important for understanding cell specification and how noncoding genetic variation contributes to disease. Enhancers have utility as part of gene therapy vectors designed to target gene expression to select cell types.

In vivo enhancer/promoter assessment has typically been performed in low-throughput single-vector assays. Effectively screening large pools of enhancers across the array of cell types in the brain or other organs would accelerate the development of useful enhancers, but faces multiple impediments. First, it is necessary to deliver the library of enhancers to a large enough population of cells to ensure the recovery of quantitative expression information from both abundant and rare cell populations. Second, each enhancer needs to be scored for specificity across different cell types. Third, enhancer interference must be obviated, as enhancers co-administered to individual cells can interfere with each other, muddling their individual activities.

Existing methods available for screening enhancers are often tedious and provide inadequate solutions to these challenges. For example, while single-cell RNA sequencing enables assessments across different cell types, it is severely constrained by sparse RNA capture and the inability to assess a large library of enhancers across a sufficient number of cells. Further, executing cell type-specific cis regulatory element screening in vivo has been challenging due to gene delivery limitations, tissue heterogeneity, and technical issues related to sequence recovery.

Therefore, there is a need for improved methods and vectors for screening large pools of enhancers across cell types.

### SUMMARY OF THE INVENTION

As described below, the present invention features compositions and methods that are useful for screening gene regulatory elements for cell type-specific expression in vivo.

In one aspect, the invention of the disclosure features a vector containing a polynucleotide. The polynucleotide contains a cis regulatory element and a promoter sequence, each within a region of the polynucleotide defined by two recombinase recognition sites. Contacting the polynucleotide with a recombinase forms a mini-circle and stabilizes mRNA transcribed from the polynucleotide in a cell.

In another aspect, the invention of the disclosure features an isolated recombinant adeno-associated virus (rAAV) particle containing the polynucleotide of the above aspect, or embodiments thereof.

In another aspect, the invention of the disclosure features a polynucleotide library of cis regulatory sequences. The cis regulatory sequences in the library are each encoded by a vector of any of the above aspects, or embodiments thereof.

In another aspect, the invention of the disclosure features a composition containing the vector of any of the above aspects, or embodiments thereof, the rAAV particle of any of the above aspects, or embodiments thereof, or the polynucleotide library of the above aspect, or embodiments thereof.

In another aspect, the invention of the disclosure features, a method for screening cis regulatory elements for cell type-specific expression. The method involves administering to a subject a vector containing a polynucleotide containing the following disposed within a region of the polynucleotide defined by two recombinase recognition sites: a cis regulatory element, a promoter sequence, a barcode, and an invertible spacer sequence that is disposed within a second set of recombinase recognition sites.

In another aspect, the invention of the disclosure features a method for screening cis regulatory elements for cell type-specific expression. The method involves: contacting cells with a vector comprising a polynucleotide containing the following disposed within a region of the polynucleotide defined by two recombinase recognition sites: a cis regulatory element, a promoter sequence, a barcode, and an invertible spacer sequence that is disposed within a second set of recombinase recognition sites.

In another aspect, the invention of the disclosure features a vector containing in order from 5′ to 3′: a flippase recognition target (FRT) site, a 3′ UTR, a cis regulatory element, a promoter, a (S/W)₁₅VHDB bar code, a lox site, an invertible spacer sequence, a second lox site, and a second flippase recognition target (FRT) site.

In another aspect, the invention of the disclosure features a vector containing in order from 5′ to 3′: a flippase recognition target (FRT) site, a 3′ UTR, a cis regulatory element, a promoter, a (S/W)₁₅VHDB bar code, a lox site, an invertible spacer sequence, a second lox site, a second flippase recognition target (FRT) site, and an mRNA destabilizing element.

In another aspect, the invention of the disclosure features a vector containing in order from 5′ to 3′: a flippase recognition target (FRT) site, a cis regulatory element, a promoter, a (S/W)₁₅VHDB bar code, a lox site, an invertible spacer sequence, a second lox site, a second flippase recognition target (FRT) site, and an mRNA destabilizing element.

In another aspect, the invention of the disclosure features a method for screening cis regulatory elements for cell type-specific expression. The method involves a) administering to a rodent one or more vectors of any of the above aspects, or embodiments thereof, where the rodent expresses a Cre and a flippase (FLP). The method further involves b) sequencing mRNA from the rodent to detect the barcodes and inversion status of the invertible spacer sequences.

In any of the above aspects, or embodiments thereof, the polynucleotide contains a viral genome. In embodiments, the viral genome is an adeno-associated virus (AAV) genome or a lentivirus genome. In any of the above aspects, or embodiments thereof, the polynucleotide is an AAV genome.

In any of the above aspects, or embodiments thereof, the vector contains a viral particle containing a viral capsid. In any of the above aspects, or embodiments thereof, the polynucleotide is encapsidated by a viral capsid. In embodiments, the capsid is a recombinant adeno-associated virus (rAAV), adenovirus, lentivirus capsid. In embodiments, the pseudotype of the rAAV capsid is selected from one or more of AAV-PHP.eB. AAVF. AAV-PHP.C1. AAV-PHP.B4, AAV-PHP.B5, AAV-PHP.B6, AAV-PHP.B7, AAV-PHP.B8, AAV-PHP.C1. AAV-PHP.C2, AAV-PHP.C3, CAP-B10, and CAP-B22.

In any of the above aspects, or embodiments thereof, the cis regulatory element is an enhancer. In any of the above aspects, or embodiments thereof, the promoter is a constitutive promoter. In any of the above aspects, or embodiments thereof, the promoter is selected from one or more of the cytomegalovirus (CMV) promoter and the cytomegalovirus enhancer/chicken beta-actin/Rabbit β-globin (CAG) promoter.

In any of the above aspects, or embodiments thereof, the two recombinase recognition sites are flippase recognition target (FRT) sites.

In any of the above aspects, or embodiments thereof, the polynucleotide further contains a 3′ untranslated region (UTR) within the region defined by the two recombinase recognition sites and 5′ of the cis regulatory sequence and promoter.

In any of the above aspects, or embodiments thereof, mini-circle formation positions the 3′ UTR 3′ of the cis regulatory element and promoter sequence so that the mRNA includes the 3′ UTR. In any of the above aspects, or embodiments thereof, the 3′ UTR contains a polyadenylation signal. In any of the above aspects, or embodiments thereof, the 3′ UTR contains a Woodchuck Hepatitis Virus Posttrascriptional Regulatory Element (WPRE). In any of the above aspects, or embodiments thereof, the 3′ UTR stabilizes the mRNA transcribed from the polynucleotide in the cell. In any of the above aspects, or embodiments thereof, the 3′ UTR stabilizes the mRNA transcript in the cell.

In any of the above aspects, or embodiments thereof, the polynucleotide further contains an mRNA destabilizing element outside of the region defined by the two recombinase recognition sites and 3′ of the cis regulatory element and promoter sequence. In any of the above aspects, or embodiments thereof, the mRNA destabilizing element destabilizes the mRNA prior to mini-circle formation. In any of the above aspects, or embodiments thereof, the mRNA after mini-circle formation does not contain the mRNA destabilizing element. In any of the above aspects, or embodiments thereof, the mRNA destabilizing element contains an AU-rich element (ARE) and/or a ribozyme sequence. In embodiments, the ribozyme sequence is selected from one or more of a T3H36, a T3H37, a T3H38, a T3H39, a T3H43, a T3H44, a T3H45, a T3H47, a T3H48, a T3H49, a T3H50, and a T3H52 sequence. In embodiments, the ribozyme sequence is a T3H47 sequence.

In any of the above aspects, or embodiments thereof, the polynucleotide further encodes a reporter polypeptide that is transcribed under the control of the promoter. In embodiments, the reporter polypeptide is a fluorescent protein. In embodiments, the fluorescent protein is GFP or mScarlet.

In any of the above aspects, or embodiments thereof, the polynucleotide further contains a barcode that is transcribed under the control of the promoter. In any of the above aspects, or embodiments thereof, the barcode contains a readable sequence at least about 5, 10, 15, or 20 nucleotides in length. In any of the above aspects, or embodiments thereof, the readable sequence contains a series of S's and W's. In any of the above aspects, or embodiments thereof, at least three of the series of S's and W's contain at least three distinct position-specific nucleotides that can be used to identify an enhancer associated with the readable sequence. In any of the above aspects, or embodiments thereof, the barcode contains an additional sequence. In any of the above aspects, or embodiments thereof, the additional sequence is at least about 1, 2, 3, 4, 5, or 10 nucleotides in length. In any of the above aspects, or embodiments thereof, the additional sequence contains the nucleotide sequence VHDB. In any of the above aspects, or embodiments thereof, the additional sequence is 3′ of the readable sequence. In any of the above aspects, or embodiments thereof, the barcode has the sequence (S/W)₁₅VHDB. In any of the above aspects, or embodiments thereof, the barcode does not contain more than three consecutive S's or W's.

In any of the above aspects, or embodiments thereof, the polynucleotide further contains an invertible spacer sequence transcribed under the control of the promoter. In any of the above aspects, or embodiments thereof, the invertible spacer sequence is disposed between a second set of recombinase recognition sites, which are within the region defined by the region defined within the first two recombinase recognition sites. In any of the above aspects, or embodiments thereof, the second set of recombinase recognition sites are Lox sites. In any of the above aspects, or embodiments thereof, the Lox sites are selected from one or more of loxP, lox71, lox66, loxJT15, loxJT15 right, loxJT510, loxJT510 right, loxJTZ17 left, and loxJTZ17. In any of the above aspects, or embodiments thereof, the Lox sites contain loxJT15 and loxJTZ17.

In any of the above aspects, or embodiments thereof, the invertible spacer sequence is 3″ or 5′ of the barcode. In any of the above aspects, or embodiments thereof, the barcode is proximal to the invertible spacer sequence. In any of the above aspects, or embodiments thereof, the barcode is within 1, 2, 3, 4, 5, 10, 15, 20, or 25 nucleotides of one of the second set of recognition sites. In any of the above aspects, or embodiments thereof, the invertible spacer sequence is at least about 5, 10, 15, 20, 25, or 50 nucleotides in length. In any of the above aspects, or embodiments thereof, the invertible spacer sequence is at least about 10, 15, 20, 25, 50, 100, 200, or 1000 nucleotides in length.

In any of the above aspects, or embodiments thereof, the cell is a mammalian cell. In embodiments, the cell is a rodent cell.

In any of the above aspects, or embodiments thereof, the polynucleotide library contains at least about 10, 20, 30, 40, 50, 100, 200, 300, 400, 500, 1000, 5000, or 10000 distinct cis regulatory sequences. In any of the above aspects, or embodiments thereof, the cis regulatory sequences are enhancer sequences.

In any of the above aspects, or embodiments thereof, the composition further contains a carrier, excipient, or diluent.

In any of the above aspects, or embodiments thereof, contacting the polynucleotide with a recombinase forms a mini-circle and stabilizes mRNA transcribed from the polynucleotide in a cell.

In any of the above aspects, or embodiments thereof, the method further involves isolating RNA from an organ or tissue of the subject.

In any of the above aspects, or embodiments thereof, the organ or tissue is part of the central nervous system. In any of the above aspects, or embodiments thereof, the organ or tissue is from the cortex or the cerebellum. In any of the above aspects, or embodiments thereof, the organ or tissue contains parvalbumin (PV) interneurons, somatostatin (SST) expressing neurons, or vesicular glutamate transport (Vglut) neurons.

In any of the above aspects, or embodiments thereof, the method further involves sequencing bulk mRNA isolated from the subject. In any of the above aspects, or embodiments thereof, the method involves determining barcode sequences and/or invertible spacer sequences transcribed in the subject from the polynucleotide.

In any of the above aspects, or embodiments thereof, each cis regulatory element corresponds to a unique readable sequence.

In any of the above aspects, or embodiments thereof, the invertible spacer sequence is transcribed under the control of the promoter.

In any of the above aspects, or embodiments thereof, the method is associated with a reduction in cross-talk between the cis regulatory sequences relative to a method using vectors that do not contain the first set of two recombinase recognition sites.

In any of the above aspects, or embodiments thereof, the first set of two recombinase recognition sites are flippase recognition target (FRT) sites.

In any of the above aspects, or embodiments thereof, the method further involves detecting the inversion status of the invertible spacer sequence in mRNA from the subject. In any of the above aspects, or embodiments thereof, the method further involves detecting, with near single-cell specificity, transcription of the barcode and/or invertible spacer in the subject.

In any of the above aspects, or embodiments thereof, the polynucleotide further contains a 3′ untranslated region (UTR) within the region defined by the first set of two recombinase recognition sites and 5′ of the cis regulatory sequence and promoter.

In any of the above aspects, or embodiments thereof, mini-circle formation positions the 3′ UTR 3′ of the cis regulatory element and promoter sequence so that the mRNA transcript transcribed under the control of the promoter includes the 3′ UTR.

In any of the above aspects, or embodiments thereof, the mRNA destabilizing element destabilizes the mRNA transcript transcribed under the control of the promoter in a cell prior to mini-circle formation. In any of the above aspects, or embodiments thereof, the mRNA transcript transcribed under the control of the promoter in the cell after mini-circle formation does not contain the mRNA destabilizing element.

In any of the above aspects, or embodiments thereof, the vectors collectively contain at least about 10, 20, 30, 40, 50, 100, 200, 300, 400, 500, 1000, 5000, or 10000 distinct cis regulatory sequences. In embodiments, the cis regulatory sequences are enhancer sequences.

In any of the above aspects, or embodiments thereof, the subject is a mammal. In any of the above aspects, or embodiments thereof, the subject is a rodent that expresses a Cre and a flippase (FLP). In embodiments, the rodent is Cre line.

In any of the above aspects, or embodiments thereof, the flippase is introduced to the cell using an AAV vector.

In any of the above aspects, or embodiments thereof, the vectors contain viral particles containing viral capsids.

In any of the above aspects, or embodiments thereof, the polynucleotides are encapsidated by viral capsids. In any of the above aspects, or embodiments thereof, the capsids are recombinant adeno-associated virus (rAAV) capsids.

In any of the above aspects, or embodiments thereof, the method further involves sequencing mRNA transcribed from the vector. In any of the above aspects, or embodiments thereof, the method further involves converting mRNA from an organ or tissue of the subject to cDNA.

In any of the above aspects, or embodiments thereof, the 3′ UTR contains a Woodchuck Hepatitis Virus Posttrascriptional Regulatory Element (WPRE) and a polyadenylation signal.

In any of the above aspects, or embodiments thereof, the vector is a recombinant adeno-associated virus (rAAV) particle with a pseudotype selected from one or more of AAV-PHP.eB. AAVF. AAV-PHP.C1, AAV-PHP.B4, AAV-PHP.B5, AAV-PHP.B6, AAV-PHP.B7, AAV-PHP.B8, AAV-PHP.C1, AAV-PHP.C2, AAV-PHP.C3, CAP-B10, and CAP-B22.

In any of the above aspects, or embodiments thereof, the mRNA destabilizing element contains a ribozyme. In any of the above aspects, or embodiments thereof, the mRNA destabilizing element contains a T3H47 sequence. In any of the above aspects, or embodiments thereof, the promoter is selected from one or more of the cytomegalovirus (CMV) promoter and the cytomegalovirus enhancer/chicken beta-actin/Rabbit β-globin (CAG) promoter.

In any of the above aspects, or embodiments thereof, the vectors collectively contain at least about 10, 20, 30, 40, 50, 100, 200, 300, 400, 500, 1000, 5000, or 10000 distinct cis regulatory elements.

In any of the above aspects, or embodiments thereof, the method involves detecting with near single-cell specificity transcription of the barcode and/or invertible spacer in the rodent.

The invention provides compositions and methods that are useful for screening gene regulatory elements for cell type-specific expression in vivo. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988): The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Cre recombinase (Cre) polypeptide” is meant a polypeptide or fragment thereof having at least about 85% amino acid identity to GenBank Accession No. BAL61207.1, which is reproduced below, and having recombinase activity.

>BAL61207.1 Cre recombinase [Cre-expression vector pHVX2-cre]
(SEQ ID NO: 1)
MVQTSLLTVHQNLPALPVDATSDEVRKNLMDMFRDRQAFSEHTWKMLLSVCRSWAAWCKLNNRK
WFPAEPEDVRDYLLYLQARGLAVKTIQQHLGQLNMLHRRSGLPRPSDSNAVSLVMRRIRKENVD
AGERAKQALAFERTDFDQVRSLMENSDRCQDIRNLAFLGIAYNTLLRIAEIARIRVKDISRTDG
GRMLIHIGRTKTLVSTAGVEKALSLGVTKLVERWISVSGVADDPNNYLFCRVRKNGVAAPSATS
QLSTRALEGIFEATHRLIYGAKDDSGORYLAWSGHSARVGAARDMARAGVSIPEIMQAGGWTNV
NIVMNYIRNLDSETGAMVRLLEDGD

By “Cre recombinase (Cre) polynucleotide” is meant a polynucleotide encoding a Cre polypeptide. An exemplary Cre polynucleotide sequence is provided at GenBank Accession No. AB517728.1, which is reproduced below.

>AB517728.1:7412-8449 Cre-expression vector pHVX2-cre DNA,
complete sequence
(SEQ ID NO: 2)
ATGGTCCAAACTAGTTTACTGACCGTACACCAAAATTTGCCTGCATTACCGGTCGATGCAACGA
GTGATGAGGTTCGCAAGAACCTGATGGACATGTTCAGGGATCGCCAGGCGTTTTCTGAGCATAC
CTGGAAAATGCTTCTGTCCGTTTGCCGGTCGTGGGCGGCATGGTGCAAGTTGAATAACCGGAAA
TGGTTTCCCGCAGAACCTGAAGATGTTCGCGATTATCTTCTATATCTTCAGGCGCGCGGTCTGG
CAGTAAAAACTATCCAGCAACATTTGGGCCAGCTAAACATGCTTCATCGTCGGTCCGGGCTGCC
ACGACCAAGTGACAGCAATGCTGTTTCACTGGTTATGCGGCGGATCCGAAAAGAAAACGTTGAT
GCCGGTGAACGTGCAAAACAGGCTCTAGCGTTCGAACGCACTGATTTCGACCAGGTTCGTTCAC
TCATGGAAAATAGCGATCGCTGCCAGGATATACGTAATCTGGCATTTCTGGGGATTGCTTATAA
CACCCTGTTACGTATAGCCGAAATTGCCAGGATCAGGGTTAAAGATATCTCACGTACTGACGGT
GGGAGAATGTTAATCCATATTGGCAGAACGAAAACGCTGGTTAGCACCGCAGGTGTAGAGAAGG
CACTTAGCCTGGGGGTAACTAAACTGGTCGAGCGATGGATTTCCGTCTCTGGTGTAGCTGATGA
TCCGAATAACTACCTGTTTTGCCGGGTCAGAAAAAATGGTGTTGCCGCGCCATCTGCCACCAGC
CAGCTATCAACTCGCGCCCTGGAAGGGATTTTTGAAGCAACTCATCGATTGATTTACGGCGCTA
AGGATGACTCTGGTCAGAGATACCTGGCCTGGTCTGGACACAGTGCCCGTGTCGGAGCCGCGCG
AGATATGGCCCGCGCTGGAGTTTCAATACCGGAGATCATGCAAGCTGGTGGCTGGACCAATGTA
AATATTGTCATGAACTATATCCGTAACCTGGATAGTGAAACAGGGGCAATGGTGCGCCTGCTGG
AAGATGGCGATTAG

By “Flp recombinase (FLP) polypeptide” is meant a polypeptide or fragment thereof having at least about 85% amino acid identity to Genbank Accession No. AAT08996.1, which is reproduced below, and having recombinase activity.

(SEQ ID NO: 3)
>AAT08996.1 Flp recombinase [Flp expression vector pFLP3]
MPQFGILCKTPPKVLVRQFVERFERPSGEKIALCAAELTYLCWMITHNGTAIKRATFMSYNTII
SNSLSFDIVNKSLQFKYKTQKATILEASLKKLIPAWEFTIIPYYGQKHQSDITDIVSSLQLQFE
SSEEADKGNSHSKKMLKALLSEGESIWEITEKILNSFEYTSRFTKTKTLYQFLFLATFINCGRF
SDIKNVDPKSFKLVQNKYLGVIIQCLVTETKTSVSRHIYFFSARGRIDPLVYLDEFLRNSEPVL
KRVNRTGNSSSNKQEYQLLKDNLVRSYNKALKKNAPYSIFAIKNGPKSHIGRHLMTSFLSMKGL
TELTNVVGNWSDKRASAVARTTYTHQITAIPDHYFALVSRYYAYDPISKEMIALKDETNPIEEW
QHIEQLKGSAEGSIRYPAWNGIISQEVLDYLSSYINRRI

By “Flp recombinase (FLP) polynucleotide” is meant a polynucleotide encoding a Flp polypeptide. An exemplary Flp polynucleotide sequence is provided at GenBank Accession No. AY597273.1, which is reproduced below.

>AY597273.1:6054-7325 Flp expression vector pFLP3, complete sequence
(SEQ ID NO: 4)
TTATATGCGTCTATTTATGTAGGATGAAAGGTAGTCTAGTACCTCCTGTGATATTATCCCATTC
CATGCGGGGTATCGTATGCTTCCTTCAGCACTACCCTTTAGCTGTTCTATATGCTGCCACTCCT
CAATTGGATTAGTCTCATCCTTCAATGCTATCATTTCCTTTGATATTGGATCATATGCATAGTA
CCGAGAAACTAGTGCGAAGTAGTGATCAGGTATTGCTGTTATCTGATGAGTATACGTTGTCCTG
GCCACGGCAGAAGCACGCTTATCGCTCCAATTTCCCACAACATTAGTCAACTCCGTTAGGCCCT
TCATTGAAAGAAATGAGGTCATCAAATGTCTTCCAATGTGAGATTTTGGGCCATTTTTTATAGC
AAAGATTGAATAAGGCGCATTTTTCTTCAAAGCTTTATTGTACGATCTGACTAAGTTATCTTTT
AATAATTGGTATTCCTGTTTATTGCTTGAAGAATTGCCGGTCCTATTTACTCGTTTTAGGACTG
GTTCAGAATTCCTCAAAAATTCATCCAAATATACAAGTGGATCGATCCTACCCCTTGCGCTAAA
GAAGTATATGTGCCTACTAACGCTTGTCTTTGTCTCTGTCACTAAACACTGGATTATTACTCCC
AGATACTTATTTTGGACTAATTTAAATGATTTCGGATCAACGTTCTTAATATCGCTGAATCTTC
CACAATTGATGAAAGTAGCTAGGAAGAGGAATTGGTATAAAGTTTTTGTTTTTGTAAATCTCGA
AGTATACTCAAACGAATTTAGTATTTTCTCAGTGATCTCCCAGATGCTTTCACCCTCACTTAGA
AGTGCTTTAAGCATTTTTTTACTGTGGCTATTTCCCTTATCTGCTTCTTCCGATGATTCGAACT
GTAATTGCAAACTACTTACAATATCAGTGATATCAGATTGATGTTTTTGTCCATAGTAAGGAAT
AATTGTAAATTCCCAAGCAGGAATCAATTTCTTTAATGAGGCTTCCAGAATTGTTGCTTTTTGC
GTCTTGTATTTAAACTGGAGTGATTTATTGACAATATCGAAACTCAGCGAATTGCTTATGATAG
TATTATAGCTCATGAATGTGGCTCTCTTGATTGCTGTTCCGTTATGTGTAATCATCCAACATAA
ATAGGTTAGTTCAGCAGCACATAATGCTATTTTCTCACCTGAAGGTCTTTCAAACCTTTCCACA
AACTGACGAACAAGCACCTTAGGTGGTGTTTTACATAATATACCAAATTGTGGCAT.

By “administering” is meant giving, supplying, dispensing a composition, agent, therapeutic product, and the like to a subject, or applying or bringing the composition and the like into contact with the subject. Administering or administration may be accomplished by any of a number of routes, such as, for example, without limitation, parenteral or systemic, intravenous (IV), (injection), subcutaneous, intrathecal, intracranial, intramuscular, dermal, intradermal, inhalation, rectal, intravaginal, topical, oral, subcutaneous, intramuscular, or intraocular. In embodiments, administration is systemic, such as by inoculation, injection, or intravenous injection.

By “agent” is meant any small molecule chemical compound, antibody, nucleic acid molecule, or polypeptide, or fragments thereof. A non-limiting example of an agent is a selection vector of the present disclosure.

By “alteration” is meant a change in the expression levels or activity of a gene or polypeptide as detected by standard art known methods such as those described herein. The alteration can be an increase or a decrease. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising.” “containing” and “having” and the like can have the meaning ascribed to them in U.S. Patent law and can mean “includes,” “including,” and the like: “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “cis regulatory element (CRE)” is meant a polynucleotide sequence that operates in part, or in whole, to regulate expression of a gene on the same strand of DNA as the CRE. Non-limiting cis regulatory elements include promoters, enhancers, silencers, and operators. Enhancers are cis regulatory elements that influence the transcription of genes on the same molecule of DNA. Enhancer may be localized upstream, downstream, within introns, or even relatively far away from a gene they regulate. In some instances, a cis regulatory element is a cis-regulatory RNA element (e.g., an iron response element, a frameshift element, or a riboswitch). Typically, the binding of a DNA binding protein(s) or transcription factor(s) to an enhancer alters transcription of an associated gene.

In this disclosure, IUPAC degenerate base symbol notation may be used, along with standard IUPAC notation for nucleobases (e.g., “A” for adenine, “C” for cytosine, “G” for guanine. “T” for thymine, etc.). IUPAC degenerate base symbol notation is explained, for example, in Cornish-Bowden A. “Nomenclature for incompletely specified bases in nucleic acid sequences: recommendations 1984.” Nucleic Acids Res. 1985 May 10; 13 (9): 3021-30. In some embodiments, “S”, as used herein, is the bases G or C. In some embodiments, “W”, as used herein, is the bases A or T. In some embodiments, “V”, as used herein, is the bases G, C, or A. In some embodiments, “H”, as used herein, is the bases A, C, or T. In some embodiments, “D”, as used herein, is the bases G, A, or T. In some embodiments, “B”, as used herein, is the bases G, T, or C.

Enhancers have been described as clusters of DNA sequences capable of binding combinations of transcription factors that then interact with components of the mediator complex or TFIID to help recruit RNA polymerase II (RNAPII). To accomplish this, enhancer-bound transcription factors loop out the intervening sequences and contact the promoter region of a gene, thus allowing enhancers to act in a distance-independent fashion. In addition, activation of eukaryotic genes requires de-compaction of the chromatin fiber, which is carried out by enhancer-bound transcription factors that can recruit histone modifying enzymes or ATP-dependent chromatin remodeling complexes to alter chromatin structure and increase the accessibility of the DNA to other proteins. For a review of enhancer function, see, e.g., Ong. C.-T. and Corces, V. G., 2011, Nat. Rev. Genetics, 12 (4): 283-293, which is incorporated herein by reference.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence, or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “flippase” is meant a recombinase which recognizes a flippase recognition target (“FRT”). An exemplary flippase is the Flp recombinase (FLP) polypeptide, as described herein.

By “fragment” is meant a portion of a polypeptide or nucleic acid molecule. This portion contains at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “gene” is meant a region of a polynucleotide that is transcribed as a single unit. Typically, a gene is transcribed to produce a single RNA molecule.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increase” is meant to alter positively by at least 5% relative to a reference. An increase may be by 5%, 10%, 25%, 30%, 50%, 75%, or even by 100%.

The terms “isolated,” “purified.” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector: into an autonomously replicating plasmid or virus: or into the genomic DNA of a prokaryote or eukaryote: or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a developmental state, condition, disease, or disorder.

By “mini-circle” is meant a small circular polynucleotide. In some embodiments, mini-circles are generated from vectors of the present disclosure, preferably through recombination events, such as those produced by the recombinases described herein. Exemplary mini-circles are about 3 kb, 4 kb, or 5 kb in size.

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “promoter” is meant a polynucleotide sequence sufficient to drive transcription of a downstream protein.

As used herein, the term “pseudotyped” refers to a viral vector that contains one or more foreign viral structural proteins. Non-limiting examples of foreign viral structural proteins include envelope glycoproteins. A pseudotyped virus may be one in which the envelope glycoproteins of an enveloped virus or the capsid proteins of a non-enveloped virus originate from a virus that differs from the source of the original virus genome and the genome replication apparatus. (D. A. Sanders, 2002, Curr. Opin. Biotechnol., 13:437-442). The foreign viral envelope proteins of a pseudotyped virus can be utilized to alter host tropism or to increase or decrease the stability of the virus particles. Examples of pseudotyped viral vectors include a virus that contains one or more envelope glycoproteins that do not naturally occur on the exterior of the wild-type virus. Pseudotyped viral vectors can infect cells and express and produce proteins. RNA transcripts, and/or molecules encoded by polynucleotides, e.g., reporter or effector proteins or molecules, contained within the viral vectors.

By “recombinase” is meant enzymes that catalyse site-specific recombination events within polynucleotides. Recombination events may involve strand breakage, strand exchange between homologous segments, and rejoining.

The term “recombinant” as used herein in the context of proteins or nucleic acids refers to proteins or nucleic acids that do not occur in nature or in a naturally occurring protein or nucleic acid sequence, but are the product of human engineering, often or typically utilizing molecular biological or molecular genetic tools and techniques practiced by the skilled practitioner in the art. For example, in some embodiments, a recombinant protein or nucleic acid molecule comprises an amino acid or nucleotide sequence that comprises at least one, at least two, at least three, at least four, at least five, at least six, at least seven, or at least eight mutations as compared to any naturally occurring sequence.

By “reduce” is meant to alter negatively by at least 5% relative to a reference. A reduction may be by 5%, 10%, 25%, 30%, 50%, 75%, or even by 100%.

By “reference” is meant a standard or control condition. In embodiments, a reference is a cell or animal that does not express a particular recombinase (e.g., Cre or FLP). In some embodiments, the reference is a cell or animal that has not been contacted with or administered a screening vector. A further non-limiting example of a reference is a cis regulatory element lacking activity or having low activity in a target cell.

A “reference sequence” is a defined sequence used as a basis for sequence comparison. A reference sequence may be a subset of or the entirety of a specified sequence: for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that can be transcribed into an mRNA molecule or that encodes a polypeptide of the invention or a fragment thereof. In embodiments, the mRNA contains a sequence corresponding to a barcode and/or invertible spacer of the present disclosure. In embodiments, nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl. G. M. and S. L. Berger (1987) Methods Enzymol. 152:399; Kimmel, A. R. (1987) Methods Enzymol. 152:507). In some instances, the nucleic acid molecule encodes a polypeptide that is not endogenous to a target cell or animal. In some cases, the nucleic acid molecule encodes corresponds to a screening vector or a fragment thereof.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C. more preferably of at least about 37° C. and most preferably of at least about 42° C. Varying additional parameters, such as hybridization time, the concentration of detergent. e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature.

As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C. more preferably of at least about 42° C. and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology. Wiley Interscience. New York, 2001): Berger and Kimmel (Guide to Molecular Cloning Techniques, 1987. Academic Press. New York); and Sambrook et al., Molecular Cloning: A Laboratory Manual. Cold Spring Harbor Laboratory Press. New York.

By “substantially identical” is meant a polypeptide or nucleic acid molecule exhibiting at least 50% identity to a reference amino acid sequence or nucleic acid sequence. In embodiments, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine: valine, isoleucine, leucine: aspartic acid, glutamic acid, asparagine, glutamine: serine, threonine: lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “stabilizes” means increases the stability of a polynucleotide relative to a reference polynucleotide. In embodiments, a stabilized mRNA is maintained for a longer period of time in a cell relative to a reference mRNA (e.g., unstabilized). In other embodiments, a stabilized RNA is maintained for weeks (e.g., 1, 3, 6, 10 weeks), months (e.g., 1, 2, 3, 4, 6, 8, 10, or 11 months), or years (e.g., 1, 2, 3, 4, 5) longer than a reference mRNA.

By “subject” is meant an organism. In embodiments, the organism is a mammal. Non-limiting examples of a subject include a human or non-human mammal, such as a non-human primate (e.g., a marmoset), or a non-human mammal, such as a bovine, equine, canine, ovine, or feline mammal, or a sheep, goat, llama, camel, or a rodent (rat, mouse), ferret, gerbil, hamster, or zebrafish. In embodiments, the subject expresses one or more recombinases, such as FLP and/or Cre.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“Transduction” refers to a process by which a polynucleotide is introduced or transferred into a cell. In embodiments, a cell is transduced by a virus or viral vector. In embodiments, the transduced polynucleotide (e.g., RNA, DNA) is expressed in the transduced cell.

As used herein, the term “vehicle” refers to a solvent, diluent, or carrier component of a pharmaceutical composition.

By “viral genome” is meant a polynucleotide molecule suitable for encapsidation by a viral capsid. A non-limiting example of a viral genome is a polynucleotide (e.g., single-stranded DNA) containing and/or flanked by two adeno-associated virus inverted terminal repeats (ITR's). In some cases, a viral genome contains a rep open reading frame and/or a cap open reading frame. In embodiments, the viral capsid is an adeno-associated virus capsid or a lentivirus capsid. In various instances, the viral genome is of sufficient size for encapsidation by a viral capsid (e.g., less than 4.7 kilobases long).

Unless specifically stated or obvious from context, as used herein, the term “or” is understood to be inclusive. Unless specifically stated or obvious from context, as used herein, the terms “a”. “an”, and “the” are understood to be singular or plural.

Unless specifically stated or obvious from context, as used herein, the term “about” is understood as within a range of normal tolerance in the art, for example within 2 standard deviations of the mean. Unless otherwise clear from context, all numerical values provided herein are modified by the term about.

In any of the above aspects, or embodiments thereof, the cells form part of an organoid or virtual organ. In any of the above aspects, or embodiments thereof, the cells contain two or more different cell types.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1G provide schematics, bar graphs, a chart, and histograms showing development and validation of a Cre-based high-throughput enhancer screening system. Cre-lox recombination enables within and between Cre line scoring of enhancer activity in vivo. FIG. 1A provides a schematic showing the basic design of the screening system. Features are indicated in the inset boxes. FIG. 1B provides a schematic highlighting how the screening system can assess the specificity and activity of different enhancers by next generation sequencing (NGS) of barcoded and Cre-tagged mRNAs. In FIGS. 1A and 1B “BC” represents “barcode” and “poly A” represents a “long chain of adenine nucleotides.” FIGS. 1C and 1D provide bar graphs showing the number of total and inverted unique BCs for each enhancer when AAV-PHP.eB carrying either a DLX (FIG. 1C) or E2 (FIG. 1D) driven barcode library was injected into the indicated Cre line and RNA was recovered for inversion rate analysis. The number of total unique barcodes (light bars) or inverted, unique barcodes (filled in bars) are shown. Adult mice were injected with a vector containing a DLX (interneuron specific) or E2 (PV neuron specific) enhancer driven reporter AAV containing a Cre-invertible element (e.g., an invertible spacer sequence) adjacent to 5 (S/W) 15 (VHDB) barcode sets. The enhancer libraries were packaged into AAV-PHP.eB and delivered intravenously. Enhancers were detected in the cortex after conversion of total RNA to cDNA and amplification by PCR. FIG. 1E provides a chart showing the inter-animal specificity scores for PV vs. SST mice. PV vs. Vglut. and PV vs. WT mice for both the DLX and E2 enhancer. The larger the ratio, the more specific the enhancer is for PV cells vs. the comparison line. The system enables specificity scoring between Cre lines. The values show the relative inversion rates for DLX or E2 enhancers between several pairs of mouse lines. The relative inversion rate is calculated based on the following formula: (# of unique inverted enhancer barcodes in mouse line A/# of total unique enhancer barcodes mouse line A)/(# of unique inverted enhancer barcodes in mouse line B/# of total unique enhancer barcodes mouse line B). FIGS. 1F and 1G provide histograms showing results from an experiment undertaken to simulate a library of 1000 enhancers. Barcodes were randomly assigned to one of 1000 unique pools. Mean inversion rate within each pool was assessed and shown as a distribution. The inversion rate in each pool was highly consistent. The histograms show the distribution of the mean percentage/density of reads inverted in each pool with the DLX (FIG. 1F) or E2 (FIG. 1G) enhancer-barcoded AAVs as assessed in the indicated transgenic mouse Cre line. The enhancers were detected in the cortex after conversion of total RNA to cDNA and amplification by PCR. Adult mice were injected with a vector containing a DLX (interneuron specific) or E2 (PV neuron specific) enhancer driven reporter AAV containing a Cre-invertible element (e.g., an invertible spacer sequence) adjacent to 5 (S/W) 15 (VHDB) barcode sets. The enhancer libraries were packaged into AAV-PHP.eB and delivered intravenously. In FIGS. 1F and 1G, the rightmost peaks in each histogram from right-to-left represent PVCre and SstCre, respectively.

FIGS. 2A-2C provide schematics and bar graphs showing that the screening vector design minimized interference between co-administered enhancers. FIG. 2A provides a bar graph showing results from an experiment where the DLX-reporter and PCP2-reporter vectors were delivered alone or codelivered to ACTBFLPe mice using AAV-PHP.eB. The graph shows RT-PCR results relative to GAPDH. FIG. 2B provides a schematic showing an overview of the screening vector system. Codelivered genomes (top) can form concatemers in vivo (middle). which may result in cis effects of the enhancers on the promoters within the other AAV genomes. The screening vector system used FLP expression and strategically placed FRT sites to isolate each enhancer-barcode (BC) unit as an independently acting mini-circle. FIG. 2C provides a bar graph relating to an experiment where the DLX and PCP2 screening AAV vectors were delivered alone or co-delivered to ACTBFLPe mice using AAV-PHP.eB. The graph shows RT-PCR results relative to GAPDH.

FIGS. 3A and 3B provide schematics illustrating how AAV vectors can be built with novel AAV capsids and gene regulatory elements designed to work together.

FIG. 4 provides a schematic showing an overview of workflow for the cis regulatory element screening system.

FIG. 5 provides a schematic showing an overview of the design of the screening vector (shown as an AAV genome) for use in cis regulatory element screening. In FIG. 5, ITR represents “inverted terminal repeat.” “FRT” represents “the flippase recognition target ‘FRT3’,” which is an engineered recombination target site derived from the wild-type FRT site, “WPRE” represents “woodchuck hepatitis virus (WHP) posttranscriptional regulatory element,” “W” represents a 5′ portion of WPRE, “pA” represents a “polyadenylation signal,” “3′-UTR” represents “three prime untranslated region,” “pro” represents a “promoter,” “BC” represents “barcode,” “lox” represents “locus of X (cross)-over,” “CNS” represents “central nervous system,” “AAV” represents “adeno-associated virus,” “TSS” represents “transcription start site,” and “Cre” represents “cyclization recombinase.”

FIG. 6 provides an overview of the cis regulatory element screening system design. In FIG. 6, ITR represents “inverted terminal repeat,” “FRT” represents “the flippase recognition target ‘FRT3’,” which is an engineered recombination target site derived from the wild-type FRT site, “WPRE” represents “woodchuck hepatitis virus (WHP) posttranscriptional regulatory element,” “pA” represents a “polyadenylation signal,” “3′-UTR” represents “three prime untranslated region,” “pro” represents a “promoter,” “BC” represents “barcode,” “lox” represents “locus of X (cross)-over,” “AAV” represents “adeno-associated virus,” and “Cre” represents “cyclization recombinase.”

FIG. 7 provides a schematic depicting the use of a Cre invertible element (e.g., an invertible spacer sequence) in the screening vectors to assess expression specificity in target cell populations in total organ RNA samples. In FIG. 7 “pro” represents “promoter,” “BC” represents “barcode,” “Cre” represents “cyclization recombinase” and “lox” represents “locus of X (cross)-over.”

FIGS. 8A and 8B provide a schematic and a bar graph showing validation of the Cre invertible element (e.g., an invertible spacer sequence) by transient transfection of test plasmids in 293T/17 cells. FIG. 8A provides a schematic of the vector design. The p20 plasmid and p21 plasmid differed by the starting orientation of the spacer sequence (Sp1) between the mutant lox sites (loxJT15 and loxJTZ17). The inversion of the TI sequence containing Sp1 and the inverted regions of the lox sites was detected by qRT-qPCR using primers complementary to the regions flanked by the arrows. FIG. 8B provides a bar graph. The inverted or non-inverted transcripts detected from the GFP transgene cassette over the reads from the mScarlet cassette were measured by qRT-qPCR. In the absence of Cre there were more than 10.000×more non-inverted or inverted transcripts expressed from the p20 or the p21 plasmids. In contrast, in the presence of Cre, the ratio of inverted to non-inverted T1 sequences was nearly equivalent, regardless of starting orientation. For each pair of bars, the left bar represents the expression of non-inverted T1 and the right bar represents the expression of inverted T1. In FIG. 8A “CAG” represents the CAB promoter (cytomegalovirus (CMB) early enhancer element, the promoter, the first exon, and the first intron of the chicken beta-actin gene, and the splice acceptor of the rabbit beta-globin gene).

FIG. 9 provides a schematic showing the highly diverse and readable barcode (BC) for facilitating approximate single transduction event (i.e., single-cell) data. Data readouts from individual sequences or small numbers of barcodes are prone to artifacts. There can be about 2.65 million barcodes per enhancer. The barcodes were readable without requiring next generation sequencing to generate a lookup table. The barcodes provided a method for near single-cell/single-cell transduction event resolution from bulk data. The barcodes dramatically improved data reliability. A string of 15 (S/W) was sufficient to enable the generation of 484 barcodes differing by at least 3 bps, having no S or W runs longer than 3, and a GC content between 40-60%. Each enhancer was associated with a specific string of S/W. 15+4 bp was used, but the length can be determined by the desired diversity (5 to more than 30 nucleotides). The VHDB sequence length can also be extended to achieve greater diversity. FIG. 9 discloses SEQ ID NOS 64-66, respectively, in order of appearance.

FIG. 10 provides schematics and images showing co-administration of AAV vectors with different cis transcriptional regulatory elements resulted in cross-talk. Mice were injected with a vector containing a DLX (interneuron specific) driven RFP reporter alone (top panels), a PCP2 (Purkinje neuron specific) driven GFP reporter (middle panels), or both vectors (bottom panels). All AAV vectors were packaged into AAV-PHP.eB and administered intravenously to adult mice.

FIG. 11 provides a schematic showing how the screening vectors eliminate crosstalk caused by concatenation of AAV genomes in vivo. Flippase generated mini-circles that separated enhancers for improved signal-to-noise by allowing the cis regulatory elements to no longer be in cis. In FIG. 11. ITR represents “inverted terminal repeat.” “FRT” represents “the flippase recognition target FRT3′.” which is an engineered recombination target site derived from the wild-type FRT site. “WPRE” represents “woodchuck hepatitis virus (WHP) posttranscriptional regulatory element.” “pA” represents a “polyadenylation signal.” “pro” represents a “promoter.” “BC” represents “barcode.” “lox” represents “locus of X (cross)-over.” “AAV” represents “adeno-associated virus.” “Cre” represents “cyclization recombinase.” and “FLP” represents “flippase.”

FIG. 12 provides a schematic showing how flippase can be used to split concatenated screening vector AAV genomes back into individual units in vivo. In FIG. 12 “RFP” represents “red fluorescent protein” and “GFP” represents “green fluorescent protein.” Separating concatemers in vivo ensures that barcoded reporters act independently of other genomes. Recombinases were leveraged to isolate individual genomes as minicircles. The recombinases generate mini-circles. The screening vectors leveraged these mini-circles to break apart concatemers. Transcription was specifically read out from the mini-circles. The FLP-frt system was chosen for mini-circle generation to maintain compatibility with the Cre-based specificity scoring.

FIGS. 13A and 13B provide a schematic and a bar graph showing incorporating a ribozyme in the 3′ untranslated region (3′UTR) of a test vector dramatically reduced the amount of detected mRNA. The mRNA degradation elements minimized expression from single or concatenated genomes in their initial configuration prior to flippase recombination. FIG. 13A provides a schematic showing the design of the dual transgene test vector construct. The test degradation elements of none (negative control). AU-rich element (ARE), or T3H47 ribozyme were designed to reduce mScarlet expression. The plasmid also expresses GFP as an internal control. FIG. 13B provides a bar graph showing relative RT-qPCR levels from cells individually transfected with the vectors shown in FIG. 13A.

FIGS. 14A-14C provide schematics, scatter plots, and bar graphs showing the evaluation of the screening vectors in HEK293T cells. FLP expression increased mScarlet mRNA by >100× in vitro. Therefore, the screening vectors provided transgene expression from rAAVs that was dependent on mini-circle formation. FIG. 14A provides a schematic showing an mScarlet reporter gene in the screening vector, which is expressed in human 293T/17, cells or any suitable mammalian cell (e.g., mouse cells), only after exposure to FLPo (mouse codon-optimized FLP recombinase that is a fusion between the SV40 nuclear localization signal and a thermostable version of the Saccharomyces cerivisiae site-specific recombinase FLP). The vectors include an mRNA degradation element (T3H47) downstream of the mScarlet gene and the FRT. FIG. 14B provides scatter plots showing mScarlet and TagBFP-FLPo fluorescence 3 days post transduction, as measured by flow cytometry. Cells were transduced with 20K or 100K vector genomes (vg)/cell of AAV: mScarlet and 0) or 100K vg/cell of AAV: TagBFP-FLPo. FIG. 14C provides a bar graph showing expression of mScarlet from mini-Circle relative to GAPDH 3-4 days post transduction measured by RT-qPCR. In FIG. 14C each set of three bars corresponds from left-to-right to “no FLP,” “+CAG-TagBFP-FLPo 100K.” and “+TRE-TagBFP-FLPo 100K.” respectively. In FIG. 14A, “Enh” represents “enhancer.”

FIG. 15 provides a schematic providing an overview of a use of the screening vector to evaluate enhancer candidates in Cre x Flp mice. In FIG. 15, ITR represents “inverted terminal repeat.” “FRT” represents “the flippase recognition target ‘FRT3’,” which is an engineered recombination target site derived from the wild-type FRT site. “WPRE” represents “woodchuck hepatitis virus (WHP) posttranscriptional regulatory element.” “pA” represents a “polyadenylation signal,” “pro” represents a “promoter,” “BC” represents “barcode.” “lox” represents “locus of X (cross)-over.” “AAV” represents “adeno-associated virus,” “Cre” represents “cyclization recombinase.” “FLP” represents “flippase.” “F1” represents “forward primer1.” “R1” represents “reverse primer 1,” and “NGS” represents “next generation sequencing.”

FIG. 16 provides a representative screening vector nucleotide sequence with annotations. In FIG. 16, “ori” represents “origin of replication,” “AmpR” represents “ampicillin resistance gene.” bGHpA represents “bovine growth hormone polyadenylation signal.” “hGH poly(a)” represents “human growth hormone polyadenylation signal (polyA).” “ITR” represents “inverted terminal repeat.” “WPRE” represents “a Woodchuck Hepatitis Virus Posttrascriptional Regulatory Element (WPRE).” “FRT3” represents “flippase recognition target 3,” “mDlx” represents a promoter sequence, and “NLS” represents “nuclear localization signal.” FIG. 16 discloses the nucleotide sequence as SEQ ID NO: 5 and the amino acid sequences as SEQ ID NOS 67-68, respectively, in order of appearance.

### DETAILED DESCRIPTION OF THE INVENTION

The invention features compositions and methods that are useful for screening gene regulatory elements for cell type-specific expression in vivo.

The invention is based, at least in part, upon the generation of a high-throughput screening method capable of simultaneously evaluating the in vivo activity and specificity of hundreds or thousands of cis regulatory elements (e.g., enhancers) in the context of a recombinant adeno-associated viral (AAV) vector. Each cis regulatory element is associated with a highly diverse set of unique, readable expressed barcodes. In embodiments, to assess cis regulatory element specificity, the method leverages cell type-specific Cre transgenic lines to invert, or tag, the screening vector adjacent to the expressed mRNA barcode. By measuring the inversion ratio associated with each barcode, the on- and off-target expression of candidate enhancers can be assessed in bulk tissue RNA, approaching single-cell resolution without the low recovery and low cell number constraints associated with other single-cell RNA sequencing based approaches. Finally, among other things, this method minimizes the cross talk that occurs between enhancers in cotransduced cells by breaking up AAV genome concatemers into individual AAV genome mini-circles using FLP-mediated recombination (FLPout). The methods described herein ensure that barcode expression is driven by the intended enhancer rather than from multiple enhancers acting in cis on concatenated genomes. Together, the advances incorporated into the enhancer/cis regulatory element (CRE) screening system represent a powerful technology for testing a large number of CREs in vivo, which makes the screening vectors and methods valuable for evaluating libraries of gene regulatory activity across specific cell types in vivo.

The methods described herein include several innovative features including a broadly applicable Cre-based specificity readout, a method to virtually eliminate cross-vector interference due to concatemerization of AAV genomes, and a highly diverse barcode that enables both bulk and near single-cell expression readouts. Successful identification and validation of enhancers across the central nervous system or a protein thereof (e.g., the cerebral cortex) can be transformative for the clinical and basic research community enabling new methods for the treatment and study of diseases, disorders, development, and processes of the central nervous system.

### Screening Vectors

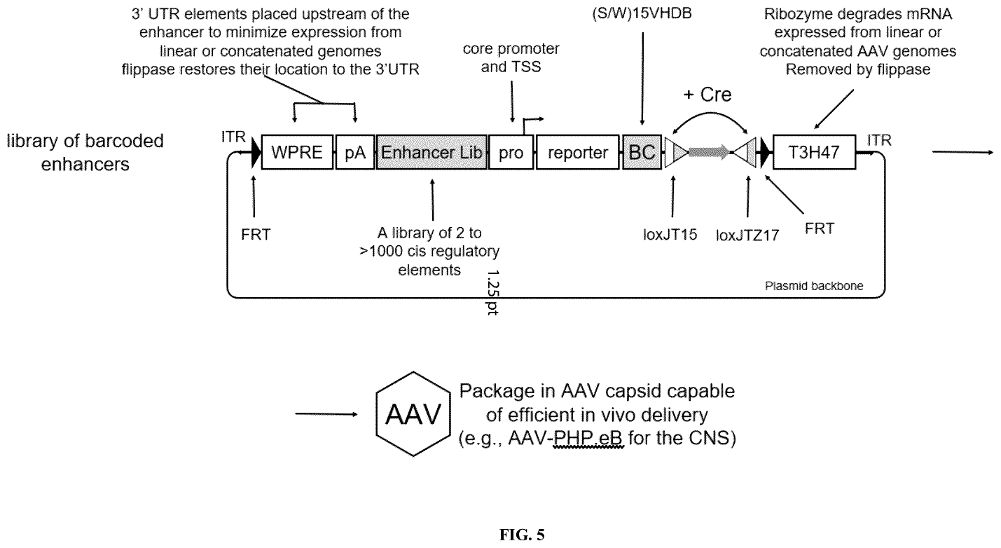

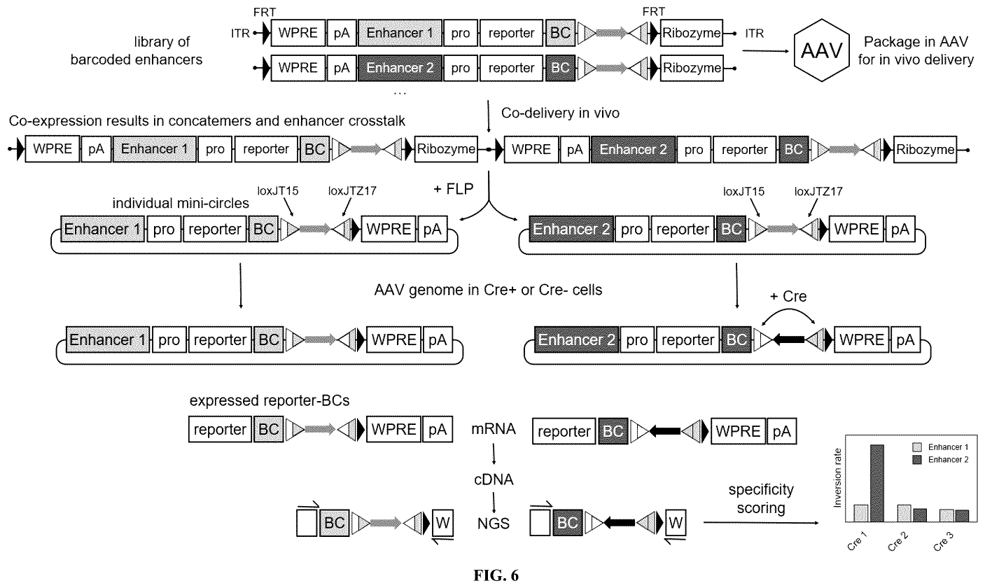

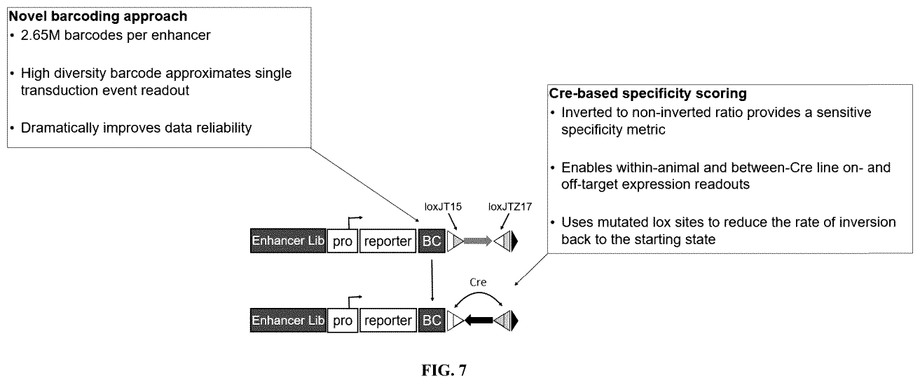

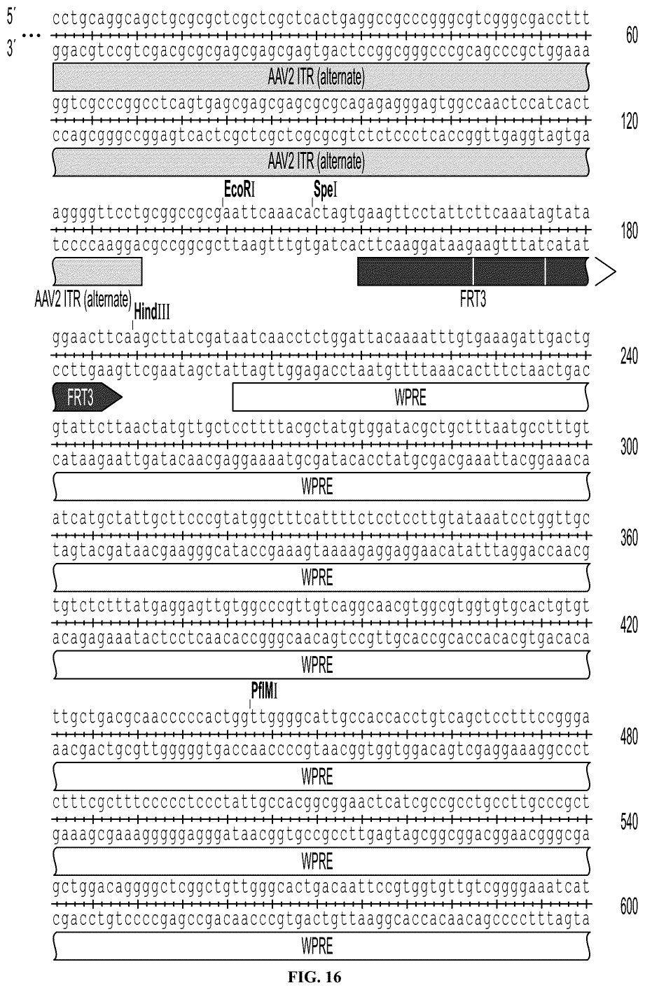

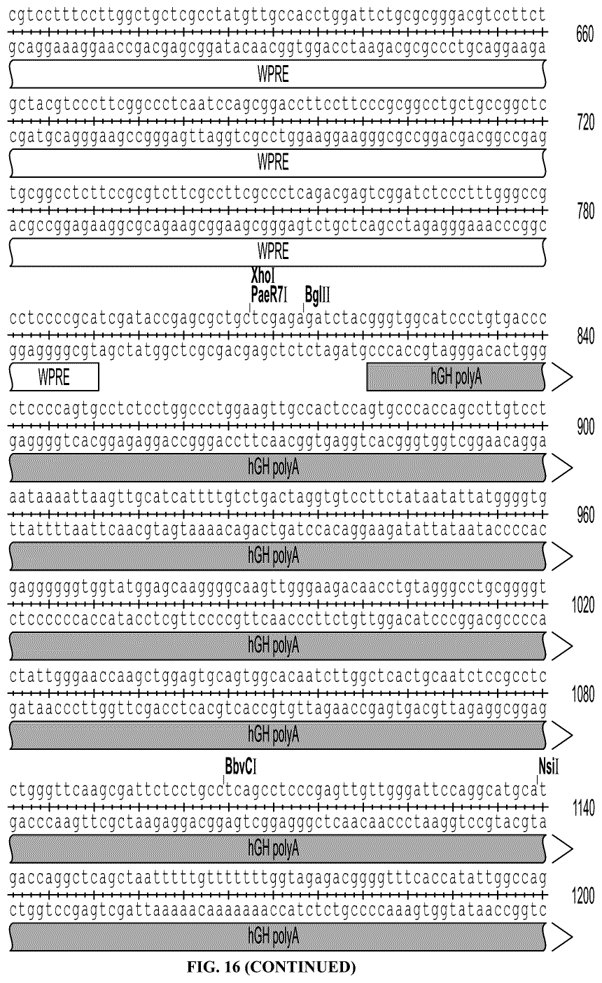

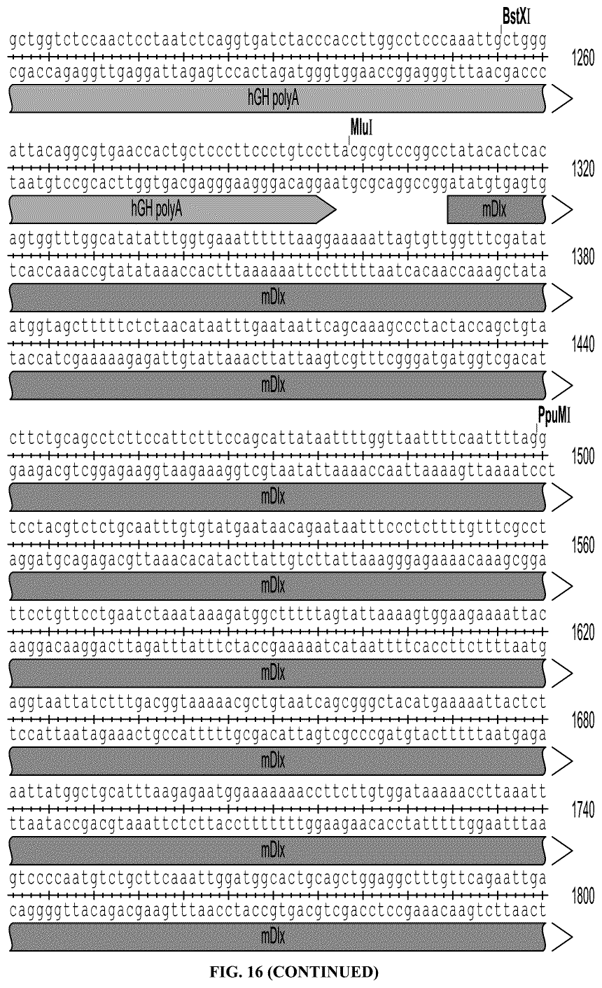

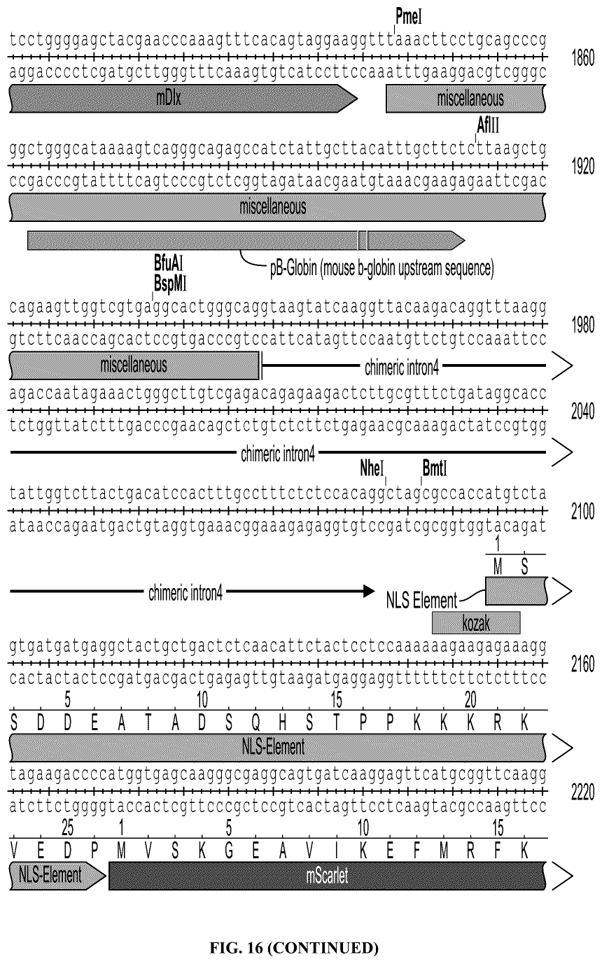

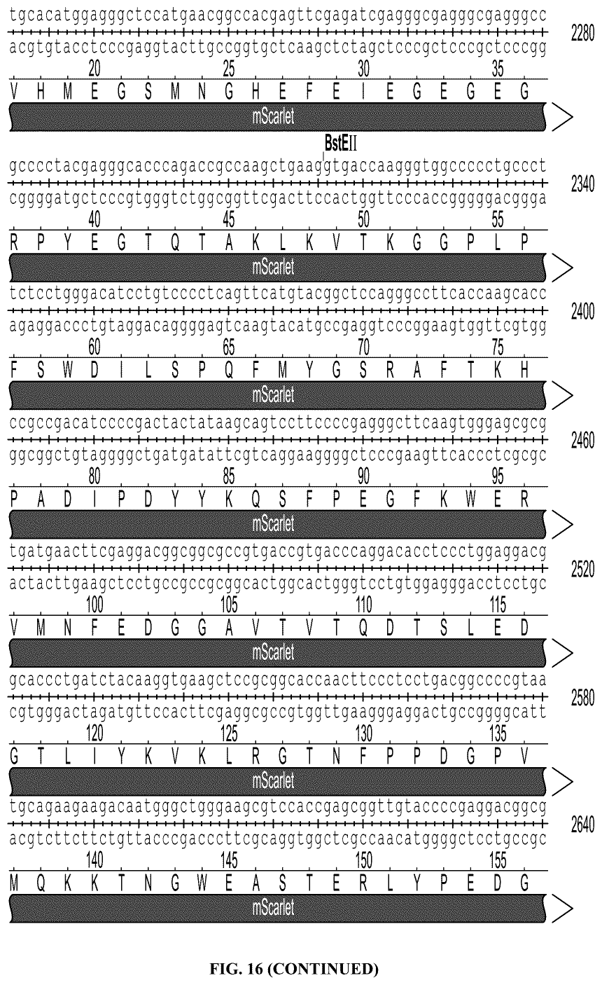

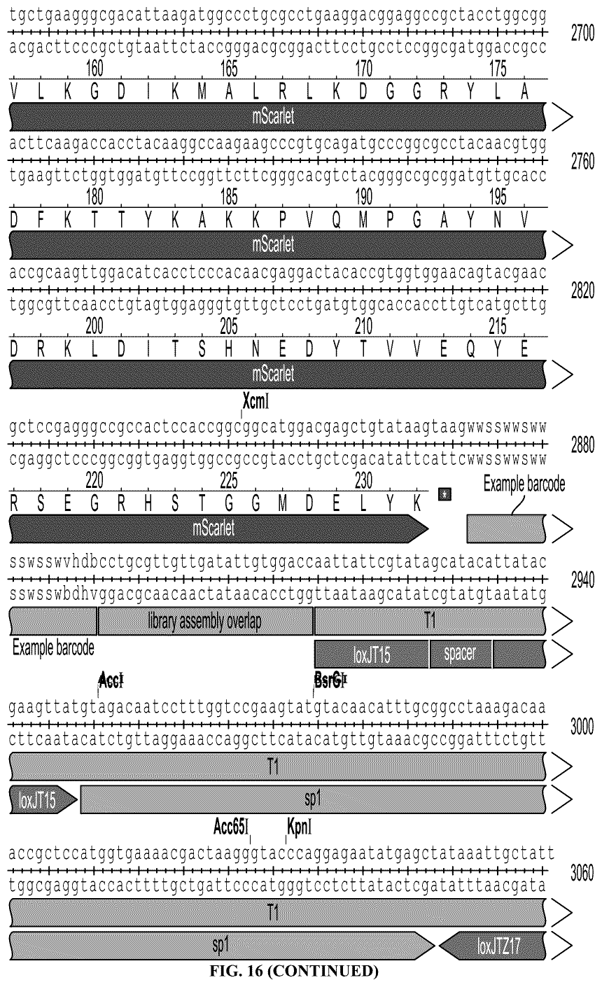

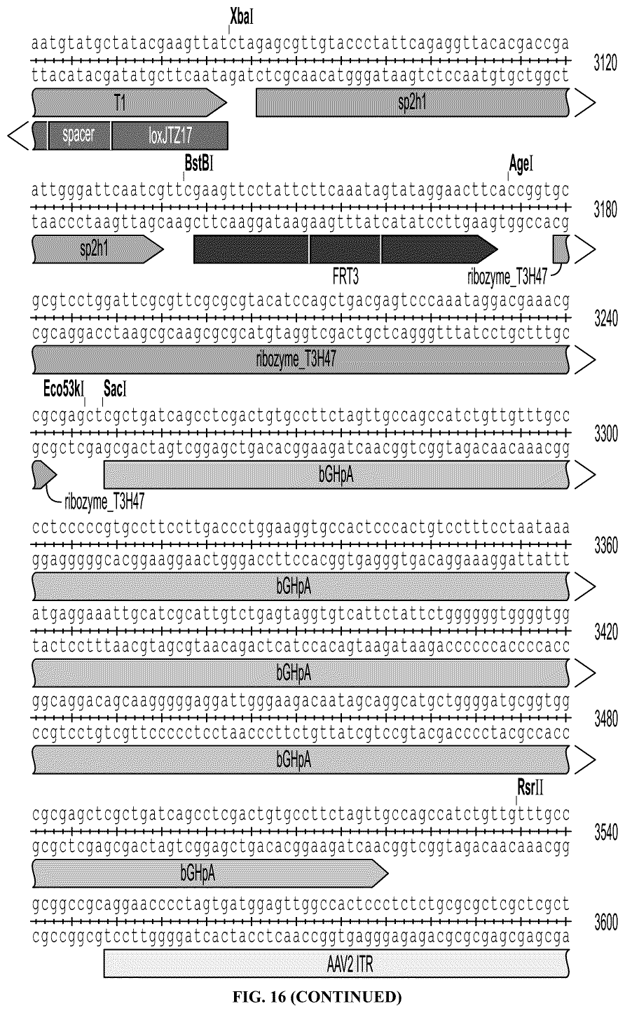

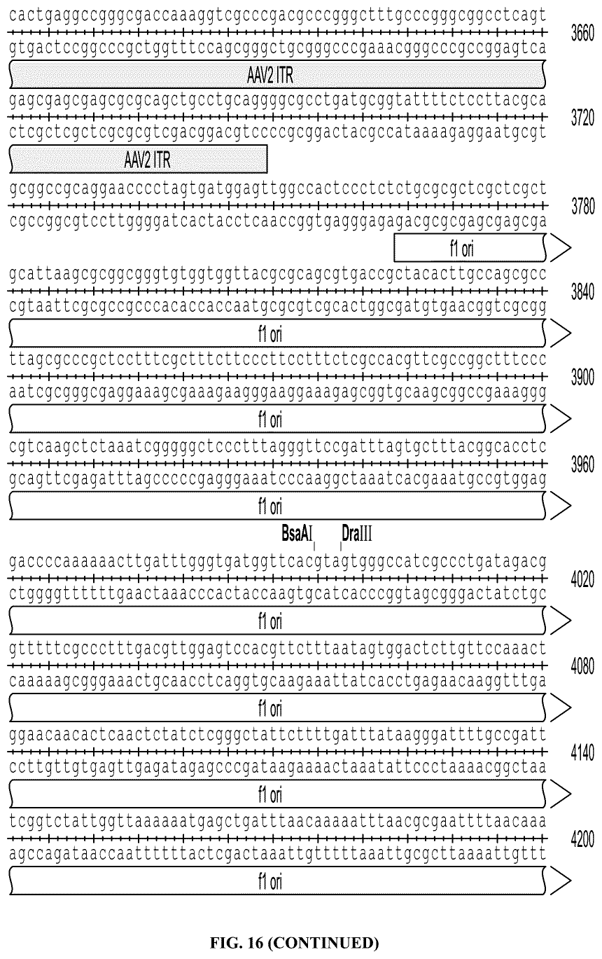

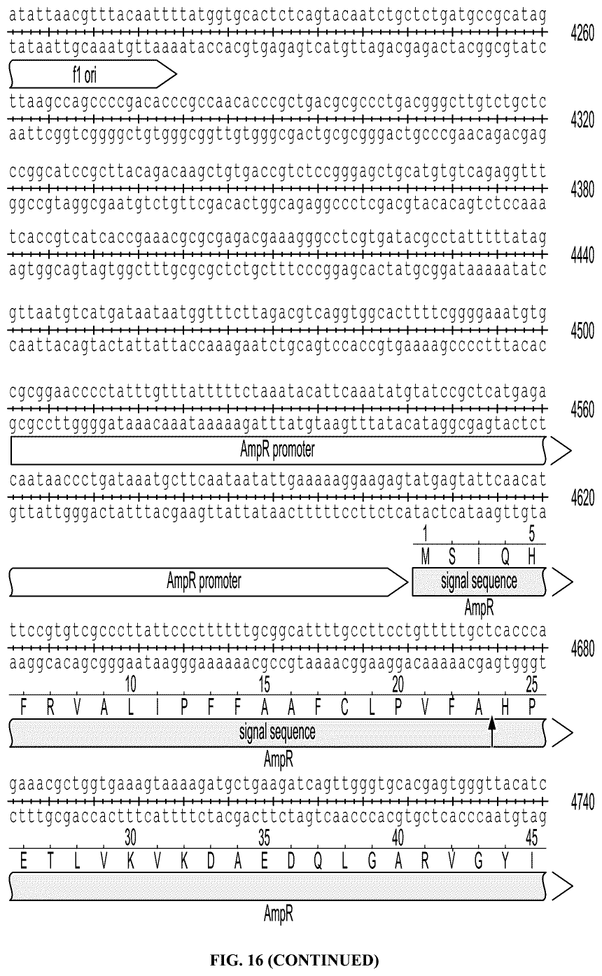

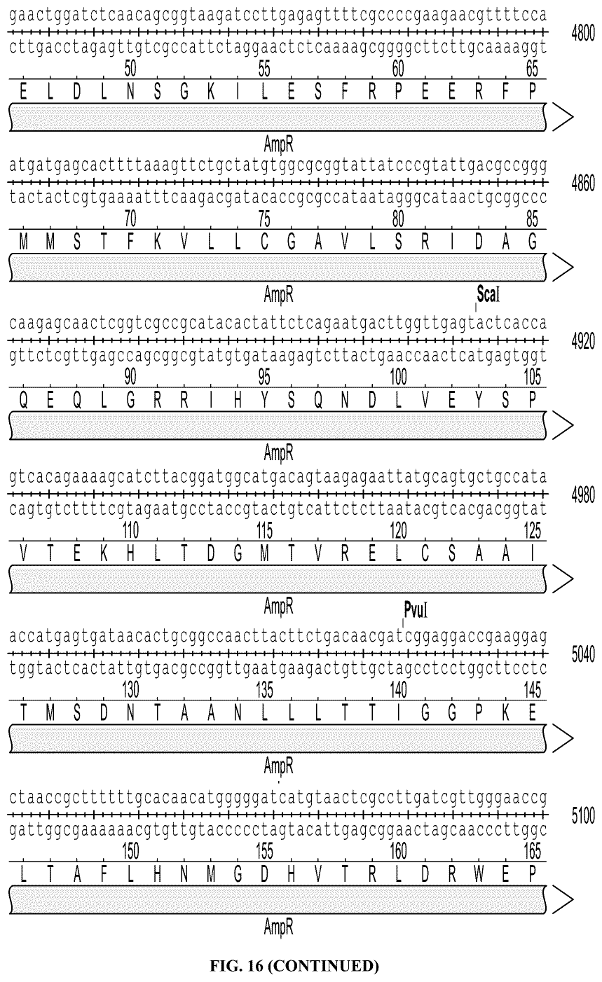

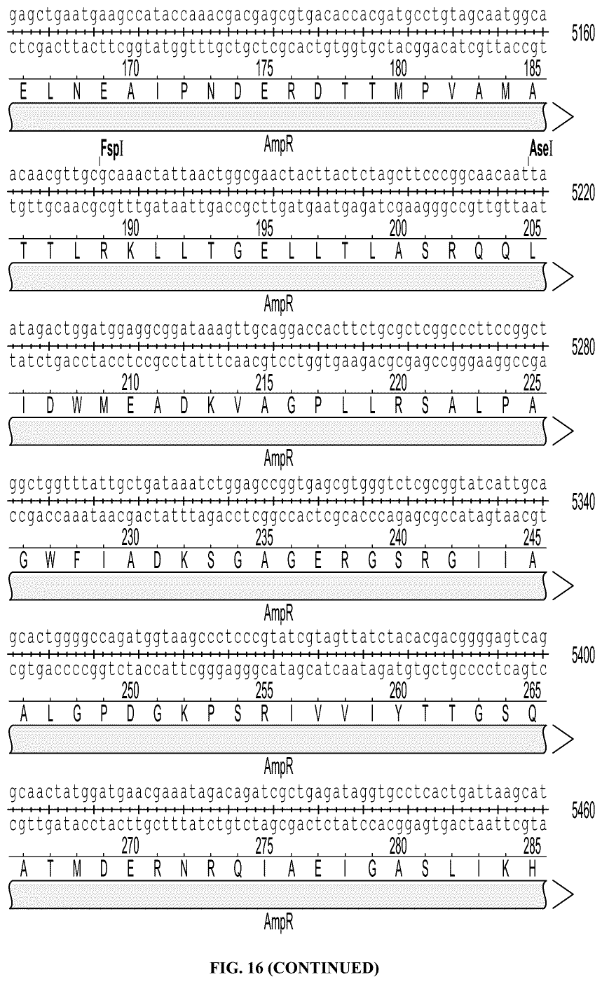

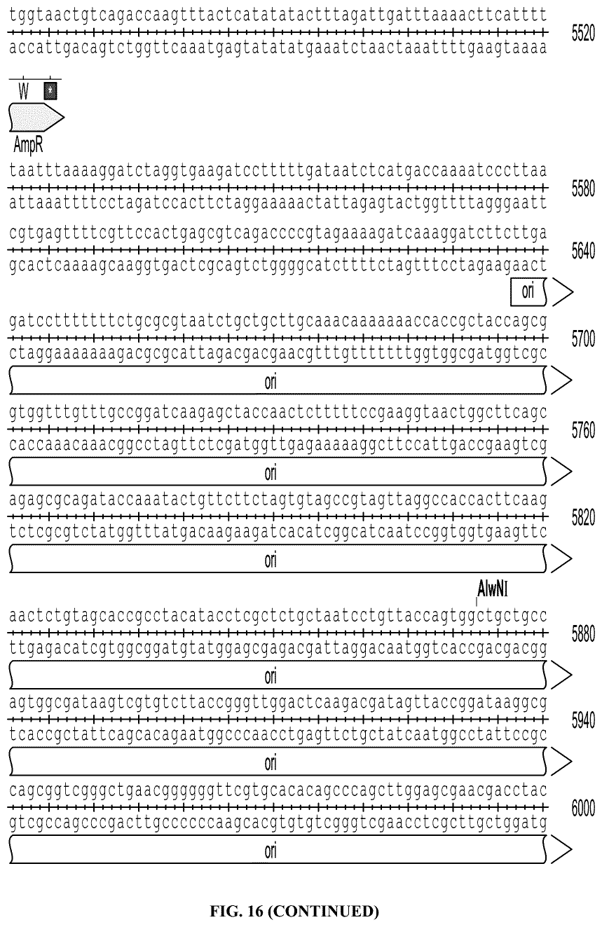

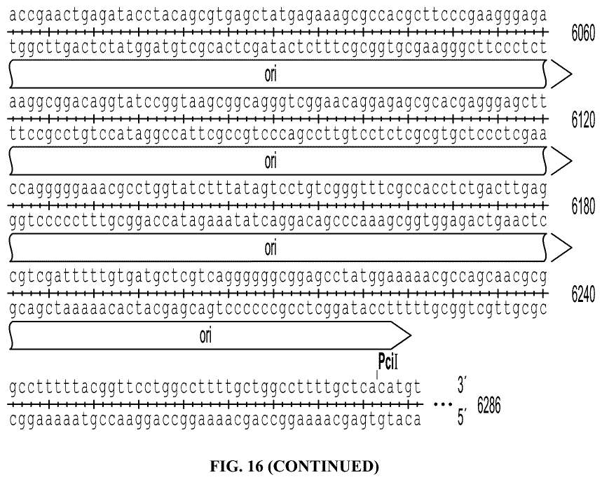

In various aspects, the invention provides vectors for screening gene regulatory elements for cell type-specific expression in vivo. A schematic presentation of an embodiment of a screening vector is provided in FIG. 5. In embodiments, the screening vectors are AAV vectors, as described further below. A representative screening vector nucleotide sequence is provided below (FIG. 16 provides a schematic of the sequence with annotations).

(SEQ ID NO: 5)
CCTGCAGGCAGCTGCGCGCTCGCTCGCTCACTGAGGCCGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTGCGGCCGCGAATTCAAACACTAGTGAAGTTCCTATTCTTCAAATAGTATAGGAACTTCAAGC
TTATCGATAATCAACCTCTGGATTACAAAATTTGTGAAAGATTGACTGGTATTCTTAACTATGT
TGCTCCTTTTACGCTATGTGGATACGCTGCTTTAATGCCTTTGTATCATGCTATTGCTTCCCGT
ATGGCTTTCATTTTCTCCTCCTTGTATAAATCCTGGTTGCTGTCTCTTTATGAGGAGTTGTGGC
CCGTTGTCAGGCAACGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCCCTATTGCCACGGCG
GAACTCATCGCCGCCTGCCTTGCCCGCTGCTGGACAGGGGCTCGGCTGTTGGGCACTGACAATT
CCGTGGTGTTGTCGGGGAAATCATCGTCCTTTCCTTGGCTGCTCGCCTATGTTGCCACCTGGAT
TCTGCGCGGGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGCCTTCGCCCTCAGACGAGTCGGATCT
CCCTTTGGGCCGCCTCCCCGCATCGATACCGAGCGCTGCTCGAGAGATCTACGGGTGGCATCCC
TGTGACCCCTCCCCAGTGCCTCTCCTGGCCCTGGAAGTTGCCACTCCAGTGCCCACCAGCCTTG
TCCTAATAAAATTAAGTTGCATCATTTTGTCTGACTAGGTGTCCTTCTATAATATTATGGGGTG
GAGGGGGGTGGTATGGAGCAAGGGGCAAGTTGGGAAGACAACCTGTAGGGCCTGCGGGGTCTAT
TGGGAACCAAGCTGGAGTGCAGTGGCACAATCTTGGCTCACTGCAATCTCCGCCTCCTGGGTTC
AAGCGATTCTCCTGCCTCAGCCTCCCGAGTTGTTGGGATTCCAGGCATGCATGACCAGGCTCAG
CTAATTTTTGTTTTTTTGGTAGAGACGGGGTTTCACCATATTGGCCAGGCTGGTCTCCAACTCC
TAATCTCAGGTGATCTACCCACCTTGGCCTCCCAAATTGCTGGGATTACAGGCGTGAACCACTG
CTCCCTTCCCTGTCCTTACGCGTCCGGCCTATACACTCACAGTGGTTTGGCATATATTTGGTGA
AATTTTTTAAGGAAAAATTAGTGTTGGTTTCGATATATGGTAGCTTTTTCTCTAACATAATTTG
AATAATTCAGCAAAGCCCTACTACCAGCTGTACTTCTGCAGCCTCTTCCATTCTTTCCAGCATT
ATAATTTTGGTTAATTTTCAATTTTAGGTCCTACGTCTCTGCAATTTGTGTATGAATAACAGAA
TAATTTCCCTCTTTTGTTTCGCCTTTCCTGTTCCTGAATCTAAATAAAGATGGCTTTTTAGTAT
TAAAAGTGGAAGAAAATTACAGGTAATTATCTTTGACGGTAAAAACGCTGTAATCAGCGGGCTA
CATGAAAAATTACTCTAATTATGGCTGCATTTAAGAGAATGGAAAAAAACCTTCTTGTGGATAA
AAACCTTAAATTGTCCCCAATGTCTGCTTCAAATTGGATGGCACTGCAGCTGGAGGCTTTGTTC
AGAATTGATCCTGGGGAGCTACGAACCCAAAGTTTCACAGTAGGAAGGTTTAAACTTCCTGCAG
CCCGGGCTGGGCATAAAAGTCAGGGCAGAGCCATCTATTGCTTACATTTGCTTCTCTTAAGCTG
CAGAAGTTGGTCGTGAGGCACTGGGCAGGTAAGTATCAAGGTTACAAGACAGGTTTAAGGAGAC
CAATAGAAACTGGGCTTGTCGAGACAGAGAAGACTCTTGCGTTTCTGATAGGCACCTATTGGTC
TTACTGACATCCACTTTGCCTTTCTCTCCACAGGCTAGCGCCACCATGTCTAGTGATGATGAGG
CTACTGCTGACTCTCAACATTCTACTCCTCCAAAAAAGAAGAGAAAGGTAGAAGACCCCATGGT
GAGCAAGGGCGAGGCAGTGATCAAGGAGTTCATGCGGTTCAAGGTGCACATGGAGGGCTCCATG
AACGGCCACGAGTTCGAGATCGAGGGCGAGGGCGAGGGCCGCCCCTACGAGGGCACCCAGACCG
CCAAGCTGAAGGTGACCAAGGGTGGCCCCCTGCCCTTCTCCTGGGACATCCTGTCCCCTCAGTT
CATGTACGGCTCCAGGGCCTTCACCAAGCACCCCGCCGACATCCCCGACTACTATAAGCAGTCC
TTCCCCGAGGGCTTCAAGTGGGAGCGCGTGATGAACTTCGAGGACGGCGGCGCCGTGACCGTGA
CCCAGGACACCTCCCTGGAGGACGGCACCCTGATCTACAAGGTGAAGCTCCGCGGCACCAACTT
CCCTCCTGACGGCCCCGTAATGCAGAAGAAGACAATGGGCTGGGAAGCGTCCACCGAGCGGTTG
TACCCCGAGGACGGCGTGCTGAAGGGCGACATTAAGATGGCCCTGCGCCTGAAGGACGGAGGCC
GCTACCTGGCGGACTTCAAGACCACCTACAAGGCCAAGAAGCCCGTGCAGATGCCCGGCGCCTA
CAACGTGGACCGCAAGTTGGACATCACCTCCCACAACGAGGACTACACCGTGGTGGAACAGTAC
GAACGCTCCGAGGGCCGCCACTCCACCGGCGGCATGGACGAGCTGTATAAGTAAGWWSSWWSWW
SSWSSWVHDBCCTGCGTTGTTGATATTGTGGACCAATTATTCGTATAGCATACATTATACGAAG
TTATGTAGACAATCCTTTGGTCCGAAGTATGTACAACATTTGCGGCCTAAAGACAAACCGCTCC
ATGGTGAAAACGACTAAGGGTACCCAGGAGAATATGAGCTATAAATTGCTATAATGTATGCTAT
ACGAAGTTATCTAGAGCGTTGTACCCTATTCAGAGGTTACACGACCGAATTGGGATTCAATCGT
TCGAAGTTCCTATTCTTCAAATAGTATAGGAACTTCACCGGTGCGCGTCCTGGATTCGCGTTCG
CGCGTACATCCAGCTGACGAGTCCCAAATAGGACGAAACGCGCGAGCTCGCTGATCAGCCTCGA
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGGCTTCTGAGGCGGAAAGAACCAGCTGGGGCTC
AGCGCTAGTGTGCGGACCGAGCGGCCGCAGGAACCCCTAGTGATGGAGTTGGCCACTCCCTCTC
TGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGCTGCCTGCAGGGGCGCCTGATGCGGTATTTTCTC
CTTACGCATCTGTGCGGTATTTCACACCGCATACGTCAAAGCAACCATAGTACGCGCCCTGTAG
CGGCGCATTAAGCGCGGCGGGTGTGGTGGTTACGCGCAGCGTGACCGCTACACTTGCCAGCGCC
TTAGCGCCCGCTCCTTTCGCTTTCTTCCCTTCCTTTCTCGCCACGTTCGCCGGCTTTCCCCGTC
AAGCTCTAAATCGGGGGCTCCCTTTAGGGTTCCGATTTAGTGCTTTACGGCACCTCGACCCCAA
AAAACTTGATTTGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACT
CTATCTCGGGCTATTCTTTTGATTTATAAGGGATTTTGCCGATTTCGGTCTATTGGTTAAAAAA
TGAGCTGATTTAACAAAAATTTAACGCGAATTTTAACAAAATATTAACGTTTACAATTTTATGG
TGCACTCTCAGTACAATCTGCTCTGATGCCGCATAGTTAAGCCAGCCCCGACACCCGCCAACAC
CCGCTGACGCGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAGACGAAAGGGC
CTCGTGATACGCCTATTTTTATAGGTTAATGTCATGATAATAATGGTTTCTTAGACGTCAGGTG
GCACTTTTCGGGGAAATGTGCGCGGAACCCCTATTTGTTTATTTTTCTAAATACATTCAAATAT
GTATCCGCTCATGAGACAATAACCCTGATAAATGCTTCAATAATATTGAAAAAGGAAGAGTATG
AGTATTCAACATTTCCGTGTCGCCCTTATTCCCTTTTTTGCGGCATTTTGCCTTCCTGTTTTTG
CTCACCCAGAAACGCTGGTGAAAGTAAAAGATGCTGAAGATCAGTTGGGTGCACGAGTGGGTTA
CATCGAACTGGATCTCAACAGCGGTAAGATCCTTGAGAGTTTTCGCCCCGAAGAACGTTTTCCA
ATGATGAGCACTTTTAAAGTTCTGCTATGTGGCGCGGTATTATCCCGTATTGACGCCGGGCAAG
AGCAACTCGGTCGCCGCATACACTATTCTCAGAATGACTTGGTTGAGTACTCACCAGTCACAGA
AAAGCATCTTACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACCGCTTTTTTGC
ACAACATGGGGGATCATGTAACTCGCCTTGATCGTTGGGAACCGGAGCTGAATGAAGCCATACC
AAACGACGAGCGTGACACCACGATGCCTGTAGCAATGGCAACAACGTTGCGCAAACTATTAACT
GGCGAACTACTTACTCTAGCTTCCCGGCAACAATTAATAGACTGGATGGAGGCGGATAAAGTTG
CAGGACCACTTCTGCGCTCGGCCCTTCCGGCTGGCTGGTTTATTGCTGATAAATCTGGAGCCGG
TGAGCGTGGGTCTCGCGGTATCATTGCAGCACTGGGGCCAGATGGTAAGCCCTCCCGTATCGTA
GTTATCTACACGACGGGGAGTCAGGCAACTATGGATGAACGAAATAGACAGATCGCTGAGATAG
GTGCCTCACTGATTAAGCATTGGTAACTGTCAGACCAAGTTTACTCATATATACTTTAGATTGA
TTTAAAACTTCATTTTTAATTTAAAAGGATCTAGGTGAAGATCCTTTTTGATAATCTCATGACC
AAAATCCCTTAACGTGAGTTTTCGTTCCACTGAGCGTCAGACCCCGTAGAAAAGATCAAAGGAT
CTTCTTGAGATCCTTTTTTTCTGCGCGTAATCTGCTGCTTGCAAACAAAAAAACCACCGCTACC
AGCGGTGGTTTGTTTGCCGGATCAAGAGCTACCAACTCTTTTTCCGAAGGTAACTGGCTTCAGC
AGAGCGCAGATACCAAATACTGTTCTTCTAGTGTAGCCGTAGTTAGGCCACCACTTCAAGAACT
CTGTAGCACCGCCTACATACCTCGCTCTGCTAATCCTGTTACCAGTGGCTGCTGCCAGTGGCGA
TAAGTCGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAGATACC
TACAGCGTGAGCTATGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAACGCCTGGTATCTT
TATAGTCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGCTGGCC
TTTTGCTCACATGT.

### Recombinase Recognition Sites and Inverted Spacer

The screening vectors contain a polynucleotide sequence containing a set of recombinase recognition sites (e.g., FRT3 sequences) defining a region of the screening vector that contains a cis regulatory element (e.g., an enhancer sequence), a barcode sequence, and an invertible spacer sequence, where the invertible spacer sequence is within a region defined by a second set of recombinase recognition sites (e.g., loxJT15 and loxJTZ17). Typically, the invertible spacer is the only polynucleotide sequence contained within the region defined by the second of recombinase recognition sites. The first set of recombinase recognition sites and the second set of recombinase recognition sites are recognized by distinct recombinases (e.g., flippase (FLP) and cyclization recombinase (Cre), respectively). Further non-limiting examples of recombinases include Dre and VCre.

The recombinase recognition sites can be selected from any of those recombinase recognition sites known in the art. Non-limiting examples of recombinase recognition sites include flippase recognition target (FRT) sequences and locos of X (cross)-over (lox) sequences. FRT and lox sequences are known in the art and it is within the skill of a practitioner to select appropriate FRT and lox sequences for use in the screening vectors (see, e.g., Tahimic, et al., “Cre/loxP, Flp/FRT systems and pluripotent stem cell lines,” Topics in Current Genetics 23:189-209 (2013): Thomson, J. G., Rucker, E. B. & Piedrahita, J. A. Mutational analysis of loxP sites for efficient Cre-mediated insertion into genomic DNA. Genesis 36, 162-167 (2003): Turan S. Galla M, Ernst E, Qiao J, Voelkel C, Schiedlmeier B, et al. (March 2011). “Recombinase-mediated cassette exchange (RMCE): traditional concepts and current challenges”. Journal of Molecular Biology. 407 (2): 193-221. Doi: 10.1016/j.jmb.2011.01.004. PMID 21241707; and Liu, et al. “Rapid pathway prototyping and engineering using in vitro and in vivo synthetic genome SCRaMblE-in methods,” Nature Communications, 9:1936 (2018), the disclosures of each of which are incorporated herein by reference in their entirety for all purposes). In some instances, the recombinase recognition sites are mutant recombinase recognition sites (e.g., loxJT15 or loxJTZ17). In embodiments, mutant recombinase recognition sites prevent double recombination events: for example, in the case of two mutant lox sites, Cre will catalyze an inversion of a polynucleotide sequence flanked by the lox sites but will be prevented from inverting the polynucleotide sequence again to place the polynucleotide sequence in its initial configuration.

Representative FRT sequence includes those sequences with at least about 85%, 90%, 95%, 99%, or 100% sequence identity to the following nucleic acid sequence, where a spacer is indicated by lowercase letters and the arms (recognition regions) flanking the spacer are in uppercase letters: GAAGTTCCTATTCtctagaaaGtATAGGAACTTC (SEQ ID NO: 6). In embodiments, the FRT sequence contains 1, 2, 3, 4, 5, 10, 20, or 25 nucleotide alterations relative to the sequence, optionally wherein the alterations are in one the spacer and/or in one or more of the recognition regions. Non-limiting examples of FRT sequences include those described in Shultz, et al., “A Genome-Wide Analysis of FRT-like Sequences in the Human Genome,” PLOS One, 6: e18077 (2011), the disclosure of which is incorporated herein by reference in its entirety for all purposes. Non-limiting examples of FRT sequences include the following, where a spacer is indicated by lowercase letters and the arms (recognition regions) flanking the spacer are in uppercase letters:

FRT1:
(SEQ ID NO: 7)
GAAGTTCCTATTCtctagataGTATAGGAACTTC;
FRT2:
(SEQ ID NO: 8)
GAAGTTCCTATTCtctacttaGTATAGGAACTTC;
FRT3:
(SEQ ID NO: 9)
GAAGTTCCTATTCttcaaataGTATAGGAACTTC;
FRT4:
(SEQ ID NO: 10)
GAAGTTCCTATTCtctagaagGTATAGGAACTTC;
FRT5:
(SEQ ID NO: 11)
GAAGTTCCTATTCttcaaaagGTATAGGAACTTC;
FRT13:
(SEQ ID NO: 12)
GAAGTTCCTATTCtcatataaGTATAGGAACTTC;
FRT14:
(SEQ ID NO: 13)
GAAGTTCCTATTCtatcagaaGTATAGGAACTTC;
FRT545:
(SEQ ID NO: 14)
GAAGTTCCTATTCtctaaaaaGTATAGGAACTTC;
mFRT11:
(SEQ ID NO: 15)
GAAGTTCCTATAGtttctagaCTATAGGAACTTC;
mFRT11-71:
(SEQ ID NO: 16)
GAAGTTTCTATAGtttctagaCTATAGAAACTTC;
and
mFRT71
(SEQ ID NO: 17)
GAAGTTTCTATTCtctagaaaGTATAGAAACTTC.

Representative Lox sequence includes those sequences with at least about 85%, 90%, 95%, 99%, or 100% sequence identity to the following nucleic acid sequence or an exemplary Lox nucleic acid sequence listed in Table 1 or Table 2 below, where a spacer is indicated by lowercase letters and the arms (recognition regions) flanking the spacer are in uppercase letters: ATAACTTCGTATAnnntannnTATACGAAGTTAT (SEQ ID NO: 18). In embodiments, the Lox sequence contains 1, 2, 3, 4, 5, 10, 20, or 25 nucleotide alterations relative to the sequence, optionally wherein the alterations are in one the spacer and/or in one or more of the recognition regions.

TABLE 1
Representative Lox sequences, where uppercase indicates wild-type bases,
lowercase indicates mutation bases, underline indicates spacer region, LE
indicates “left element,” RE indicates “right element,” and WT indicates
“wild type.”

| | Mutation | | SEQ |
|---|---|---|---|
| Lox mutant | element | Sequence | ID NO: |
| loxP | WT | ATAACTTCGTATAGCATACATTATACGAAGTTAT | 19 |
| lox71 | LE | taccgTTCGTATAGCATACATTATACGAAGTTAT | 20 |
| lox66 | RE | ATAACTTCGTATAGCATACATTATACGAAcggta | 21 |
| loxJT15 | LE | AattaTTCGTATAGCATACATTATACGAAGTTAT | 22 |
| loxJT15 right | RE | ATAACTTCGTATAGCATACATTATACGAAtaatT | 23 |
| loxJT510 | LE | taAcgTTCGTATAGCATACATTATACGAAGTTAT | 24 |
| loxJT510 right | RE | ATAACTTCGTATAGCATACATTATACGAAcgTta | 25 |
| loxJTZ17 left | LE | ATAAaTTgcTATAGCATACATTATACGAAGTTAT | 26 |
| loxJTZ17 | RE | ATAACTTCGTATAGCATACATTATAgcAAtTTAT | 27 |

TABLE 2
Representative Lox sequences.

| | 13 bp | 8 bp | 13 bp |
|---|---|---|---|
| | Recognition | Spacer | Recognition |
| Name | Region | Region | Region |
| Wild- | ATAACTTCGTATA | ATGTATGC | TATACGAAGTTAT |
| Type | (SEQ ID NO: 28) | | (SEQ ID NO: 29) |
| lox 511 | ATAACTTCGTATA | ATGTATaC | TATACGAAGTTAT |
| | (SEQ ID NO: 28) | | (SEQ ID NO: 29) |
| lox 5171 | ATAACTTCGTATA | ATGTgTaC | TATACGAAGTTAT |
| | (SEQ ID NO: 28) | | (SEQ ID NO: 29) |
| lox 2272 | ATAACTTCGTATA | AaGTATcC | TATACGAAGTTAT |
| | (SEQ ID NO: 28) | | (SEQ ID NO: 29) |
| M2 | ATAACTTCGTATA | AgaaAcca | TATACGAAGTTAT |
| | (SEQ ID NO: 28) | | (SEQ ID NO: 29) |
| M3 | ATAACTTCGTATA | taaTACCA | TATACGAAGTTAT |
| | (SEQ ID NO: 28) | | (SEQ ID NO: 29) |
| M7 | ATAACTTCGTATA | AgaTAGAA | TATACGAAGTTAT |
| | (SEQ ID NO: 28) | | (SEQ ID NO: 29) |
| M11 | ATAACTTCGTATA | cgaTAcca | TATACGAAGTTAT |
| | (SEQ ID NO: 28) | | (SEQ ID NO: 29) |
| lox 71 | TACCGTTCGTATA | NNNTANNN | TATACGAAGTTAT |
| | (SEQ ID NO: 30) | | (SEQ ID NO: 29) |
| lox 66 | ATAACTTCGTATA | NNNTANNN | TATACGAACGGTA |
| | (SEQ ID NO: 28) | | (SEQ ID NO: 31) |
| loxPsym | ATAACTTCGTATA | atgtacat | TATACGAACGGTA |
| | (SEQ ID NO: 28) | | (SEQ ID NO: 31) |

In embodiments, the first set of recombinase recognition sites are recognized by a recombinase that, when brought into contact with the screening vector, creates a mini-circle comprising the region defined within the two recombinase recognition sites (see FIG. 6). In further embodiments, the second set of recombinase recognition sites are recognized by a recombinase that, when brought into contact with the screening vector, inverts the nucleotide sequence contained within the recombinase recognition sites.

The sequence of the invertible spacer sequence is non-limiting. The spacer can be about, at least about, or no more than 5, 10, 15, 20, 25, 30, 35, 40, 45, 50, 55, 60, 65, 70, 75, 80, 85, 90, 85, 100, 200, 300, 400, 500, 600, 700, 800, 900, 1000, or more base pairs in length. Typically, the sequence of the invertible spacer sequence is not palindromic (i.e., read as the same sequence in both directions).

### Promoters

The screening vectors further comprise a promoter within the first set of recombinase recognition sites and upstream of the barcode sequence and the invertible spacer sequence, such that the promoter controls expression of an mRNA transcript transcribed from the barcode sequence and the invertible spacer sequence. In embodiments, the promoter is downstream (i.e., 3′ of) of the cis regulatory element.

Examples of suitable promoters include, but are not limited to the retroviral Rous sarcoma virus (RSV) LTR promoter (optionally with the RSV enhancer), a CAMKIIa promoter, the cytomegalovirus (CMV) promoter (optionally with the CMV enhancer) (see, e.g., Boshart et al (1985) Cell, 41:521-530), a JeT promoter, an SV40 promoter, a dihydrofolate reductase promoter, the β-actin promoter (e.g., chicken β-actin promoter), the MBP (myelin basic protein) promoter, the phosphoglycerol kinase (PGK) promoter, an EF1α promoter, an EFS promoter, a CBA promoter, UBC promoter, GUSB promoter, an NSE promoter, a Synapsin promoter, an MeCP2 (methyl-CPG binding protein 2) promoter, GFAP, a GfABC1D promoter, a CBh promoter and the like. Exemplary promoters include, but are not limited to, the MoMLV LTR, a CK6 promoter, a tyrosine hydroxylase (TH) promoter, a transthyretin promoter (TTR), a PCP2 promoter, a TK promoter, a tetracycline responsive promoter (TRE), an HBV promoter, an hAAT promoter, a LSP promoter, chimeric liver-specific promoters (LSPs), the E2F promoter, the telomerase (hTERT) promoter: the cytomegalovirus enhancer/chicken beta-actin/Rabbit β-globin promoter (CAG promoter: Niwa et al., Gene. 1991, 108 (2): 193-9) and the elongation factor 1-alpha promoter (EF1-alpha) promoter (Kim et al., Gene. 1990, 91 (2): 217-23 and Guo et al., Gene Ther., 1996, 3 (9): 802-10). In some embodiments, the promoter comprises a human β-glucuronidase promoter or a cytomegalovirus enhancer linked to a chicken β-actin (CBA) promoter. In an embodiment, the promoter is a minimal promoter, e.g., a human beta-globin minimal promoter (phβg) and a chimeric intron sequence (Hermeming et al., 2004, J Virol Methods, 122 (1): 73-77). Further examples of promoters include those described in Tornoe. J., et al., “Generation of a synthetic mammalian promoter library by modification of sequences spacing transcription factor binding sites,” Gene, 297:21-32 (2002), the disclosure of which is incorporated herein by reference in its entirety for all purposes.

The promoter can be a constitutive, inducible, or repressible promoter. The promoter can be a heatshock dependent promoters, or an interferon or NFKB responsive promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Inducible promoters and inducible systems are available from a variety of commercial sources, including, without limitation, Invitrogen, Clontech and Ariad. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (see, e.g., WO 98/10088); the ecdysone insect promoter (see, e.g., No et al, Proc. Natl. Acad. Sci. USA, 93:3346-3351 (1996)), the tetracycline-repressible system (see, e.g., Gossen et al, Proc. Natl. Acad. Sci. USA, 89:5547-5551 (1992)), the tetracycline-inducible system (see, e.g., Gossen et al, Science, 268:1766-1769 (1995), and Harvey et al, Curr. Opin. Chem. Biol., 2:512-518 (1998)), the RU486-inducible system (see, e.g., Wang et al, Nat. Biotech., 15:239-243 (1997) and Wang et al, Gene Ther., 4:432-441 (1997)) and the rapamycin-inducible system (see, e.g., Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, vectors of the present invention comprise expression control sequences imparting tissue-specific gene expression capabilities. In some cases, the tissue-specific expression control sequences bind tissue-specific transcription factors that induce transcription in a tissue specific manner. Exemplary tissue-specific regulatory sequences include, but are not limited to, the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter; alpha-fetoprotein (AFP) promoter, bone osteocalcin promoter; bone sialoprotein promoter, CD2 promoter; immunoglobulin heavy chain promoter; T cell receptor α-chain promoter, neuronal such as neuron-specific enolase (NSE) promoter, neurofilament light-chain gene promoter, and the neuron-specific vgf gene promoter. In some embodiments, the expression control sequence allows for specific expression in the central nervous system (CNS) or a subset of one or more neurons or other CNS cells.

### Reporter Gene

The screening vectors may further contain a reporter gene under the control of the promoter and cis regulatory element and upstream (i.e., 5′ of) of the barcode and invertible spacer sequence. The reporter gene can be any heterologous gene detectable by methods available to one of skill in the art. Non-limiting examples of reporter genes include fluorescent proteins, such as green fluorescent protein (GFP), mScarlet, and the like.

### Barcode

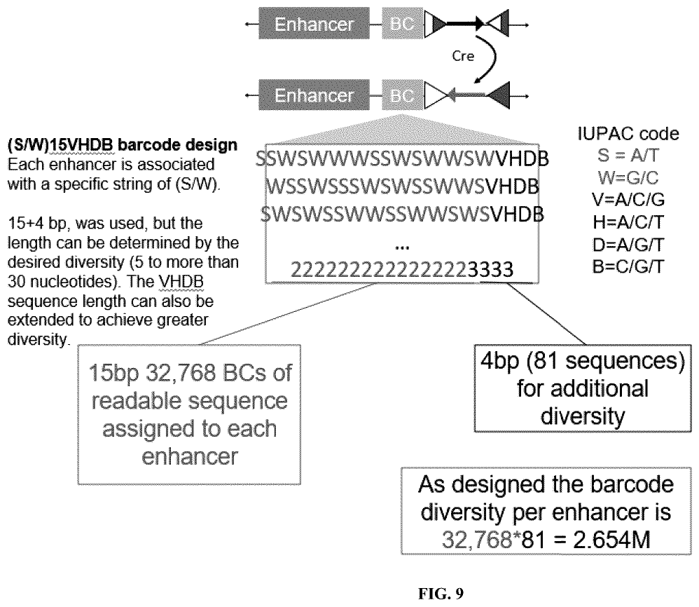

The screening vectors contain a highly diverse and readable barcode (FIG. 9). The barcode contains a readable sequence followed by an additional sequence for increasing sequence diversity. In embodiments, the readable sequence comprises about, at least about, or no more than 5, 10, 15, 20, 25, 30, 35, 40, 45, or 50 base pairs. The readable sequence comprises a series of S's (i.e, A/T) and W's (i.e., G/C). In embodiments, the series of S's and W's does not comprise more than 2, 3, 4, 5, 6, 7, 8, 9, or 10 consecutive S's or W's. In some cases, the series of S's and W's contains one or more distinct position-specific nucleobase(s) at about, at least about, or no more than about 2, 3, 4, 5, 6, 7, 8, 9, or 10 positions that can be used to identify a cis-regulatory element (e.g., an enhancer) associated with the readable sequence. These distinct position-specific nucleobases can have the advantage of allowing for assignment of a barcode to a cis regulatory element even if there is a base-pair mutation or sequencing error. The particular nucleobase(s) can be consecutive or non-consecutive. Each enhancer sequence is associated with a unique readable sequence. In embodiments, the additional sequence comprises about, at least about, or no more than about 1, 2, 3, 4, 5, 6, 7, 8, 9, 10, 15, 20, or 25 base pairs. In various embodiments, the additional sequence contains a sequence defined by one or more of V (A/C/G), H (A/C/m), D (A/G/T), and/or B (C/G/T). In some cases, the additional sequence contains the nucleotide sequence VHDB. A non-limiting example of a barcode sequence is an (S/W)15VHDB sequence containing a series of 15 S's or W's (i.e., (S/W)15) followed by the sequence VHDB.

The barcodes advantageously are readable without requiring NGS to generate a lookup table and provide over 2.65 million unique barcodes per cis regulatory sequence. Such a high number of unique barcodes, as described further below, allows for near single-cell and/or single transduction event resolution of detection of cis regulatory element activity from bulk mRNA gathered from a sample. The high diversity of barcodes also improves data reliability, as they enable assessments of the reproducibility of expression and specificity measurements within each sample. Furthermore, in embodiments, by assessing the distribution of the expression (NGS reads) per barcode, the highly diverse barcode set enables the detection of expression even from rare subpopulations of cells, which may be missed by bulk assessments of enhancer activity. In instances, the individual barcode readouts are also beneficial relative single-cell RNAseq-based screening methods where rare cell populations may only be represented a number of cells insufficient for screening a large library of unique enhancers.

The barcodes in embodiments is upstream or downstream of the invertible spacer sequence. Typically, the barcode is proximal to the invertible spacer sequence and outside of the region defined by the second set of recombinase recognition sites.

### 3′ UTR

In embodiments, the screening vector contains a 3′ UTR sequence within the region defined by the first set of recombinase recognition sites and proximal to and 3′ of the 5′ recombinase recognition site of the first set of recombinase recognition sites. In embodiments, the 3′ UTR sequence is proximal to and/or adjacent to one of the first set of recombinase recognition sites. In some instances, the enhancer and promoter sequences are 3′ of the 3′ UTR sequence (i.e., the recombination sequence and 3′UTR sequence are both 5′ of the enhancer and promoter sequences) in the screening vector.

The 3′ UTR sequence contains elements that, when transcribed as the 3′ portion of an mRNA transcript, increase the stability of the mRNA transcript. Non-limiting examples of elements suitable for inclusion in the 3′ UTR sequence include elements such as a Woodchuck Hepatitis Virus Posttrascriptional Regulatory Element (WPRE), and/or a polyadenylation signal (pA sequence), such as bovine growth hormone polyadenylation signal and/or SV40 polyomavirus simian virus 40 polyadenylation signal. In embodiments, the pA sequence is placed 5′ of the transcriptional regulatory elements (e.g., the enhancer and promoter sequences). The absence of the pA from the mRNA transcribed from the screening vector destabilizes the mRNA. In an embodiment, mini-circle formation, as described below, places the WPRE and pA sequence in their optimal location in the 3′ UTR of the mRNA transcribed under the control of the enhancer and promoter. Therefore, the positioning of the 3′ UTR in the incorrect position (i.e., in a position such that it is not transcribed under the control of the promoter) in the screening vector in its initial configuration (i.e., prior to mini-circle formation, as described below) serves to destabilize mRNA transcribed from the screening vector in the initial configuration.

mRNA Destabilizing Element

In some advantageous embodiments, the screening vectors further contain an mRNA destabilizing element downstream (i.e., 3′ of) of the 3′-most recombinase recognition site of the first set of recombinase recognition sites. In various embodiments, the mRNA destabilizing element is not included in mini-circles formed from the screening vector so that mRNA transcribed from the mini-circles does not contain the mRNA destabilizing element. In embodiments, when the screening vector is in its initial configuration or concatenated with other vectors (i.e., prior to mini-circle formation) the mRNA transcript transcribed from the vector includes the mRNA destabilizing element.

Non-limiting examples of mRNA destabilizing elements include ribozymes (e.g., T3H36, T3H37, T3H38, T3H39, T3H43, T3H44, T3H45, T3H47, T3H48, T3H49, T3H50, T3H52, or other hammerhead ribozymes) and AU-rich elements (AREs). Non-limiting examples of mRNA destabilizing elements suitable for use in the screening vectors include those disclosed in PCT/US2020/055495. Exemplary ribozymes include those disclosed in Zhong, et al. “A reversible RNA on-switch that controls gene expression of AAV-delivered therapeutics in vivo,” Nat. Biotechnol. 38:169-175 (2020), the disclosure of which is incorporated herein by reference in its entirety for all purposes. In embodiments, the ribozyme is N107, T3H1, T3H16, T3H38, or T3H48. In some embodiments, an mRNA destabilizing element comprises a microRNA site (e.g., a universal microRNA site). Exemplary ribozyme sequences are provided below and further include sequences with about or at least about 85%, 90%, or 95% sequence identity to the below sequences or fragments thereof:

T3H36:
(SEQ ID NO: 32)
GCGCGTCCTGGATTCCACTGCTTCGGCAGGTACATCCAGCTGACGAGTC
CCAAATAGGACGAAACGCGC.
T3H37:
(SEQ ID NO: 33)
GCGCGTCCTGGATTCCACTTTCGAGGTACATCCAGCTGACGAGTCCCAA
ATAGGACGAAACGCGC.
T3H38:
(SEQ ID NO: 34)
GCGCGTCCTGGATTCCACTTCGGGTACATCCAGCTGACGAGTCCCAAAT
AGGACGAAACGCGC.
T3H39:
(SEQ ID NO: 35)
GCGCGTCCTGGATTCCATTCGGTACATCCAGCTGACGAGTCCCAAATAG
GACGAAACGCGC.
T3H43:
(SEQ ID NO: 36)
GCGCGTCCTGGATTCGCATTCGCGTACATCCAGCTGACGAGTCCCAAAT
AGGACGAAACGCGC.
T3H44:
(SEQ ID NO: 37)
GCGCGTCCTGGATTCGCGATTCCGCGTACATCCAGCTGACGAGTCCCAA
ATAGGACGAAACGCGC.
T3H45:
(SEQ ID NO: 38)
GCGCGTCCTGGATTCGCGCATTCGCGCGTACATCCAGCTGACGAGTCCC
AAATAGGACGAAACGCGC.
T3H47:
(SEQ ID NO: 39)
GCGCGTCCTGGATTCGCGTTCGCGCGTACATCCAGCTGACGAGTCCCAA
ATAGGACGAAACGCGC.
T3H48:
(SEQ ID NO: 40)
GCGCGTCCTGGATTCGCGGAAACGCGTACATCCAGCTGACGAGTCCCAA
ATAGGACGAAACGCGC.
T3H49:
(SEQ ID NO: 41)
GCGCGTCCTGGATTCGCGTCACCGCGTACATCCAGCTGACGAGTCCCAA
ATAGGACGAAACGCGC.
T3H50:
(SEQ ID NO: 42)
GCGCGTCCTGGATTCGCGAGAGGAGGCCGCGTACATCCAGCTGACGAGT
CCCAAATAGGACGAAACGCGC.
T3H52:
(SEQ ID NO: 43)
GCGCGTCCTGGATTCGGCCAGAGGAGGCGGCCGTACATCCAGCTGACGA
GTCCCAAATAGGACGAAACGCGC.

### Modified Polynucleotides

In some embodiments of any of the aspects, a nucleic acid sequence as described herein is chemically modified to enhance stability or other beneficial characteristics. The nucleic acids described herein may be synthesized and/or modified by methods such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages nucleic acids having modified backbones include, among others, those that do not have a phosphorus atom in the backbone.

Modified nucleic acids that do not have a phosphorus atom in their internucleoside backbone can also be considered to be oligonucleosides. In some embodiments, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2- [known as a methylene (methylimino) or MMI backbone], —CH2-O—N(CH3)-CH2-, —CH2-N(CH3)-N(CH3)-CH2- and —N(CH3)-CH2-CH2- [wherein the native phosphodiester backbone is represented as —O—P—O—CH2-].

In other nucleic acid mimetics, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The nucleic acid can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et ah, (2005) Nucleic Acids Research 33(1):439-447; Mook, O R. et ak, (2007) Mol. Cane. Ther. 6(3):833-843; Grunweller, A. et ah, (2003) Nucleic Acids Research 31(12):3185-3193).

Modified nucleic acids can also contain one or more substituted sugar moieties. The nucleic acids described herein can include one of the following at the 2′ position: OH; F; O-, S-, or N-alkyl; O-, S-, or N-alkenyl; O-, S- or N-alkynyl; or O-alkyl-O-alkyl, where the alkyl, alkenyl and alkynyl may be substituted or unsubstituted C1 to CIO alkyl or C2 to CIO alkenyl and alkynyl. Exemplary suitable modifications include O[(CH2)nO] mCH3, O(CH2)nOCH3, O(CH2)nNH2, O(CH2) nCH3, O(CH2)nONH2, and O(CH2)nON[(CH2)nCH3)]2, where n and m are from 1 to about 10. In some embodiments, nucleic acids include one of the following at the 2′ position: C1 to CIO lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid, or a group for improving the pharmacodynamic properties of a nucleic acid, and other substituents having similar properties. In some embodiments, the modification includes a 2′ methoxyethoxy (2′-O—CH2CH2OCH3, also known as 2′-O-(2-methoxyethyl) or 2′-MOE) (Martin et al, Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O-CH2-O-CH2-N(CH2)2).

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. “Unmodified” or “natural” nucleobases include the purine bases adenine (A) and guanine (G), and the pyrimidine bases thymine (T), cytosine (C) and uracil (U). Modified nucleobases can include other synthetic and natural nucleobases including but not limited to as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments, modified nucleobases can include d5SICS and dNAM, which are a non-limiting example of unnatural nucleobases that can be used separately or together as base pairs (see e.g., Leconte et. al. J. Am. Chem. Soc. 2008, 130, 7, 2336-2343; Malyshev et. al. PNAS. 2012, 109 (30) 12005-12010). In some embodiments, oligonucleotide tags (e.g., Oligopaint) comprise any modified nucleobases known in the art, i.e., any nucleobase that is modified from an unmodified and/or natural nucleobase.

The preparation of the modified nucleic acids, backbones, and nucleobases described above are known in the art.

Another modification of a nucleic acid featured in the disclosure involves chemically linking to a polynucleotide one or more ligands, moieties or conjugates that enhance the activity, cellular distribution, pharmacokinetic properties, or cellular uptake of the polynucleotide. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4: 1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et ak, EMBO J, 1991, 10: 1111-1118; Kabanov et al., LEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

### Adeno-Associated Virus (AAV)

AAV is a small (25 nm), nonenveloped virus that contains a linear single-stranded DNA genome packaged into the viral capsid. AAV belongs to the family Parvoviridae and is of the genus Dependovirus. Productive infection by AAV occurs only in the presence of either an adenovirus or herpesvirus helper virus. In the absence of helper virus, AAV (serotype 2) can establish latency after transduction into a cell by specific but rare integration into chromosome 19q13.4. Accordingly, AAV is the only mammalian DNA virus known to be capable of site-specific integration. (Daya, S. and Berns, K. I., 2008, Clin. Microbiol. Rev., 21(4):583-593). There are two stages to the AAV life cycle after successful infection: a lytic stage and a lysogenic stage. In the presence of adenovirus or herpesvirus helper virus, the lytic stage persists. During this period, AAV undergoes productive infection characterized by genome replication, viral gene expression, and virion production. The adenoviral genes that provide helper functions for AAV gene expression include E1a, E1b, E2a, E4, and VA RNA. While adenovirus and herpesvirus provide different sets of genes for helper function, they both regulate cellular gene expression and provide a permissive intracellular milieu for a productive AAV infection. Herpesvirus aids in AAV gene expression by providing viral DNA polymerase and helicase as well as the early functions necessary for HSV transcription.

In the absence of adenovirus or herpesvirus, AAV replication is limited; viral gene expression is repressed; and the AAV genome can establish latency by integrating into a 4-kb region on chromosome 19 (q13.4), called AAVS1. The AAVS1 locus is near several muscle-specific genes, TNNT1 and TNNI3. The AAVS1 region itself is an upstream part of the gene MBS85 whose product has been shown to be involved in actin organization. Tissue culture experiments suggest that the AAVS1 locus is a safe integration site.

AAV has attracted considerable interest as a vector for use in polynucleotide delivery to subjects due to a number of desirable features. Chief amongst these is the virus's lack of pathogenicity. AAV can also infect non-dividing cells and has the ability to stably integrate into the host cell genome at a specific site (designated AAVS1) in the human chromosome 19. A desired gene together with a promoter to drive transcription of the gene can be inserted between the inverted terminal repeats (ITRs) that aid in concatemer formation in the nucleus after the single-stranded vector DNA is converted by host cell DNA polymerase complexes into double-stranded DNA. Non-integrating AAV-based polynucleotide therapy vectors typically form episomal concatemers in the host cell nucleus. In non-dividing cells, these concatemers remain intact for the life of the host cell. In dividing cells, non-integrating AAV DNA is lost through cell division, since the episomal DNA is not replicated along with the host cell DNA. As a viral vector, AAV can be used to deliver myriad polynucleotides to a subject and/or a population of cells or different cell types.

Recombinant AAV (rAAV) for Delivery of Screening Vectors

The disclosure provides for recombinant adeno-associated virus (rAAV) particles (alternatively, “AAV vectors”) containing the screening vectors. In embodiments, the screening vectors are rAAV genomes.

AAVs are well suited for use as vectors and vehicles for gene transfer cells. AAVs provide safe, long-term expression in a cell (e.g., a nerve cell). AAV vectors have been highly successful in fulfilling all of the features desired for a delivery vehicle, such as the ability to attach to and enter the target cell, successful transfer to the nucleus, the ability to be expressed in the nucleus for a sustained period of time, and a general lack of pathogenicity and toxicity. Recombinant AAV (rAAV) is advantageous as a delivery vector, particularly for delivery to the central nervous system, as it is focally injectable; it exhibits stable expression over time; and it is both non-pathogenic and non-integrative into the genome of the cell into which it is transduced. Twelve human serotypes of AAV (AAV serotype 1 (AAV-1) to AAV-12) and more than 100 serotypes from nonhuman primates have been reported to date. (Daya, S. and Berns, K. I., 2008, Clin. Microbiol. Rev., 21(4):583-593). In addition, rAAV has been approved by the FDA for use as a vector in at least 38 protocols for several different human clinical trials. AAV's lack of pathogenicity, persistence and its many available serotypes have increased the potential of the virus as a delivery vehicle for a gene therapy application in accordance with the described compositions and methods.

In embodiments, the screening vectors can be encapsidated by AAV-PHP.B (see, e.g., Deverman, et al. “Cre-dependent selection yields AAV variants for widespread gene transfer to the adult brain,” Nat Biotechnol. 2016 February; 34(2):204-209. PMCID: PMC5088052, the disclosure of which is incorporated herein by reference in its entirety for all purposes), an AAV-PHP.eB (described in Deverman B E, Pravdo P L, Simpson B P, Kumar S R, Chan K Y, Banerjee A, Wu W-L, Yang B, Huber N, Pasca S P, Gradinaru V. Cre-dependent selection yields AAV variants for widespread gene transfer to the adult brain. Nat Biotechnol. 2016 February; 34(2):204-209. PMCID: PMC5088052; and Chan K Y, Jang M J, Yoo B B, Greenbaum A, Ravi N, Wu W-L, Sánchez-Guardado L, Lois C, Mazmanian S K, Deverman B E, Gradinaru V. Engineered AAVs for efficient noninvasive gene delivery to the central and peripheral nervous systems. Nat Neurosci. 2017 August; 20(8):1172-1179. PMCID: PMC5529245), AAVF (described in Hanlon K S, Meltzer J C, Buzhdygan T, Cheng M J, Sena-Esteves M, Bennett R E, Sullivan T P, Razmpour R, Gong Y, Ng C, Nammour J, Maiz D, Dujardin S, Ramirez S H, Hudry E, Maguire C A. Selection of an Efficient AAV Vector for Robust CNS Transgene Expression. Mol Ther Methods Clin Dev. 2019 Dec. 13; 15:320-332. PMCID: PMC6881693, the disclosure of which is incorporated herein by reference in its entirety for all purposes), AAV-PHP.B4-B8, AAV-PHP.C1-C3 (Kumar, S. R. et al. Multiplexed Cre-dependent selection yields systemic AAVs for targeting distinct brain cell types. Nat Methods 17, 541-550 (2020), 9P31) or other capsids with similar properties (Nonnenmacher, M. et al. Rapid Evolution of Blood-Brain Barrier-Penetrating AAV Capsids by RNA-Driven Biopanning. Mol Ther—Methods Clin Dev (2020) doi:10.1016/j.omtm.2020.12.006), or CAP-B10 or CAP-B22 (Goertsen, D. et al. AAV capsid variants with brain-wide transgene expression and decreased liver targeting after intravenous delivery in mouse and marmoset. Nat Neurosci 1-10 (2021) doi:10.1038/s41593-021-00969-4). Further non-limiting examples of AAV capsids suitable for encapsidation of the screening vectors of the disclosure include those described in PCT/US2019/044796, PCT/US2020/027708, PCT/US2020/044487, or PCT/US2020/015972, the disclosures of each of which are incorporated herein by reference in their entireties for all purposes.

In some instances, the screening vector is encapsidated by a blood-brain barrier crossing AAV capsid. In various embodiments, the methods of the invention involve delivering an enhancer library broadly to a host using an intravenously administered AAV capsid encapsidating the screening vectors. In some cases, the screening vectors are encapsidated by and delivered to a cell using the AAV-PHP.eB capsid. The AAV capsids can be combined with the screening vectors to allow for the evaluation of specificity and strength of each or a subset of a library of enhancers across multiple cell populations in vivo. In other embodiments, the screening vector could be encapsidated in a capsid suitable for efficient, broad expression after direct delivery into the brain or other target organ.

Recombinant AAV (rAAV) vectors have been constructed with genomes that do not encode the replication (Rep) proteins and that lack the cis-active, 38 base pair integration efficiency element (IEE), which is required for frequent site-specific integration. The inverted terminal repeats (ITRs) are retained because they are the cis signals required for packaging. Thus, current screening vectors delivered using AAV capsids (i.e., as AAV vectors) persist primarily as extrachromosomal elements.

AAV-2-based rAAV vectors can transduce muscle, liver, brain, retina, and lungs, requiring several days to weeks for optimal expression. The efficiency of rAAV transduction is dependent on the efficiency at each step of AAV infection, i.e., virus binding, entry, trafficking, nuclear entry, uncoating, and second-strand synthesis.

Recombinant AAV vectors can be made using standard and practiced techniques in the art and employing commercially available reagents. In some embodiments, plasmid vectors may encode all or some of the well-known replication (rep), capsid (cap) and adeno-helper components. The rep component comprises four overlapping genes encoding Rep proteins required for the AAV life cycle (e.g., Rep78, Rep68, Rep52 and Rep40). The cap component comprises overlapping nucleotide sequences of capsid proteins VP1, VP2 and VP3, which interact together to form a capsid of an icosahedral symmetry. A second plasmid that encodes helper components and provides helper function for the AAV vector may also be co-transfected into cells. Non-limiting examples of helper components include the adenoviral genes E2A, E4orf6, and VA RNAs for viral replication.

In an embodiment, a method of making rAAVs for the products, compositions, and uses described herein involves culturing cells that comprise an rAAV polynucleotide expression vector (e.g., a polynucleotide containing a screening vector); culturing the cells to allow for expression of the polynucleotides to produce the rAAVs within the cell, and separating or isolating the rAAVs from cells in the cell culture and/or from the cell culture medium. Such methods are known and practiced by those having skill in the art. The rAAVs can be purified from the cells and cell culture medium to any desired degree of purity using conventional techniques.

Recombinant AAV vectors, which have a genome of small size (about 5 kb), can be engineered to package and contain larger genomes (transgenes), e.g., those that are greater than 4.7 kb. By way of example, two approaches developed to package larger amounts of genetic material (genes, polynucleotides, nucleic acid) include split AAV vectors and fragment AAV (fAAV) genome reassembly (Hirsch, M. L. et al., 2010, Mol Ther 18(1):6-8; Hirsch, M. L. et al., 2016, Methods Mol Biol. 1382:21-39).

An advantage and benefit of the vectors, compositions and methods described herein is their use in the identification of enhancer elements (cis-acting elements) that are capable of specifically restricting gene expression to a defined population of cells.

### Cell-Specific AAV Capsids

The rational design of AAV vectors that display selective tissue/organ targeting has broadened the applications of AAV as vector/vehicle for polynucleotide delivery to cells. Both direct and indirect targeting approaches have been used to enhance AAV vector cell targeting specificity and retargeting. By way of example, in direct targeting, AAV vector targeting to certain cell types is mediated by small peptides or ligands that have been directly inserted into the viral capsid sequence. This approach has been successfully employed to target endothelial cells. Direct targeting requires detailed knowledge of the capsid structure such that peptides or ligands are positioned at sites that are exposed to the capsid surface; the insertion does not significantly affect capsid structure and assembly; and the native tropism is ablated to maximize targeting to a specific cell type. In indirect targeting, AAV vector targeting is mediated by an associating molecule that interacts with both the viral surface and the specific cell surface receptor. Such associating molecules for AAV vectors may include bispecific antibodies and biotin. The advantages of indirect targeting are that different adaptors can be coupled to the capsid without resulting in significant changes in the capsid structure, and the native tropism can be easily ablated. A disadvantage of using adaptors for targeting involves a potential for decreased stability of the capsid-adaptor complex in vivo.

In addition, AAV vectors may be produced that comprise capsids that allow for the increased transduction of cells and gene transfer to the central nervous system and the brain via the vasculature (Chan, K. Y. et al., 2017, Nat. Neurosci., 20(8):1172-1179). Such vectors facilitate robust transduction of neuronal cells, including interneurons. In embodiments, AAV vectors contain an AAVF, AAV-PHP.B4, AAV-PHP.B5, AAV-PHP.C1, 9P31, or an AAV-PHP.eB capsid.

### Delivery of Recombinant Adeno-Associated Viral Vectors

For direct delivery to the brain, rAAV vectors may be administered by open neurosurgical procedure or by focal injection in order to bypass the blood-brain barrier, to temporally and spatially restrict transgene expression, and to target specific areas of the brain, e.g., interneuron cells and brain tissue comprising these cells.

Systemic rAAV delivery (by intravenous injection) provides a non-invasive alternative for broad gene delivery to the nervous system. Several groups have developed rAAV capsids that enhance gene transfer to the CNS and certain tissues and cell populations after intravenous delivery. By way of example, AAV-AS capsid18 utilizes a polyalanine N-terminal extension to the AAV9.4719 VP2 capsid protein to provide higher neuronal transduction, particularly in the striatum. The AAV-BR1 capsid20, based on AAV2, may be useful for more efficient and selective transduction of brain endothelial cells. Another AAV capsid, AAV-PHP.B, comprises a capsid that transduces the majority of neurons and astrocytes across many regions of the adult mouse brain and spinal cord after intravenous injection.

Other modes of rAAV vector administration may include lipid-mediated vector delivery, hydrodynamic delivery, and a gene gun.

The virus vectors and compositions thereof as described herein may be used to screen libraries of cis regulatory elements to identify cis regulatory elements that have specificity or particular activity levels in particular cell types.

### Screening Assays

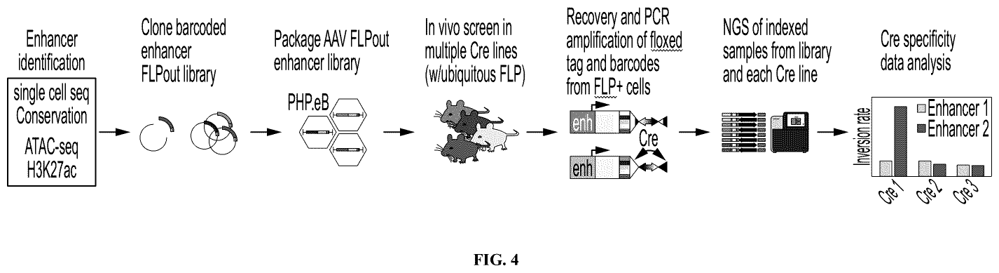

In various aspects, the present disclosure provides methods for screening cis regulatory elements using the screening vectors (e.g., AAV vectors) of the invention. Schematics showing embodiments of the screening methods are provided in FIGS. 4, 6, 11, and 15.

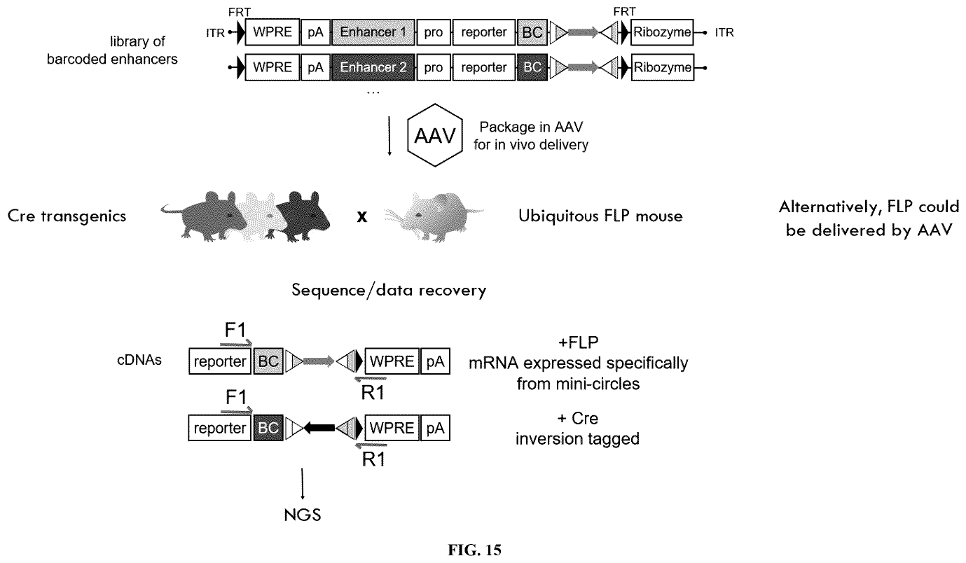

In embodiments, the screening methods involve preparing libraries of cis regulatory elements (e.g., enhancer elements) using the screening vectors provided herein (see, e.g., FIG. 15). The screening vectors are packaged in AAV capsids and then delivered to Cre transgenic mice that express FLP, or an alternative pairing of appropriate recombinases. The recombinases can be selected so that they do not interfere with one another's activity or interfere with the proper functioning of the screening vectors in the screening method. The FLP can be introduced to the mice through any suitable method, such as through cross-breeding or using a vector (e.g., an AAV vector). An FLP gene can be introduced to the mice concurrently with the screening vectors. Typically, FLP (flippase) is expressed in all cells in which the enhancers are screened. After infecting the mice with the screening AAV vectors, total mRNA is isolated from cells, tissues, and/or organs of interest and sequenced. In embodiments, barcodes and their associated invertible spacers are sequenced and analyzed to measure activity of the cis regulatory elements in the Cre transgenic mouse. The methods of the invention can advantageously provide a highly sensitive screening approach that is scalable across as much tissue (and as much RNA) as is necessary to quantitatively assess expression and specificity, even in rare cell types. In embodiments, the methods of the invention do not rely on single-cell sequencing of polynucleotides (e.g., single-cell RNA sequencing) In embodiments, the methods of the invention do not involves single-cell sequencing of polynucleotides. In some instances, the methods provide for near single-cell level specificity and expression scoring. Advantageously, expression specificity scoring can be done from bulk RNA without any need for single-cell isolation.

In embodiments, the methods of the invention involve measuring specificity and/or expression of one or more cis regulatory elements in about, or at least about, 10 cells, 100 cells, 10,000 cells, 1e5 cells, 1e6 cells, 1e7 cells, 1e8 cells, 1e9 cells, or more.

In embodiments, the methods of the present disclosure provide multiple complementary metrics of specificity: 1) the total number of unique barcodes sequenced (inverted and total); 2) the number of inverted and total barcode reads assigned to each cis regulatory element; and/or 3) the ratio of inverted to total (inversion rate), which can be calculated form the number of unique barcodes or the number of barcode reads. In embodiments the methods of the present invention provide for: 1) determining cis regulatory element specificity from bulk RNA samples, even in rare cell populations; 2) inter- and intra-animal specificity scores; and/or 3) specificity scores relative to reference elements (e.g., CAG, hSYN, and/or DLX).

In various embodiments, the methods of the invention involve scoring of the specificity of one or more cis regulatory elements in individual samples (e.g., in a cell type and/or subject) and/or between samples (e.g., between different cell types and/or subjects) using a ratio of inversion rates for the one or more cis regulatory elements (i.e., inversion ratios or relative inversion rates). In embodiments, the methods of the invention involve calculating a ratio of inversion rates (i.e., an inversion ratio or relative inversion rate) for one or more enhancers in a sample (e.g., between different cell types in a subject) or between samples (e.g., between different subjects or cell types). In embodiments, an enhancer is considered as more active in a sample than a second enhancer if the inversion ratio of the first enhancer to the second enhancer is about or at least about 2, 3, 4, 5, 10, 20, 30, 40, 50, 60, 70, 80, 90, 100, 200, 300, 400, 500, 600, 700, 800, 900, 1000.

Methods for preparation of Cre mice are known in the art, and various Cre mice are available to one of skill in the art (see, e.g., Kim, et al., “Mouse Cre-LoxP system: general principles to determine tissue-specific roles of target genes,” Lab Anim Res, 34:147-159 (2018), the disclosure of which is incorporated herein by reference in its entirety for all purposes.) In embodiments, the screening method is used to screen about, at least about, or no more than about 1, 2, 3, 4, 5, 10, 20, 30, 40, 50, 75, 100, 200, 300, 400, 500, 600, 700, 800, 900, 1000, 2000, 3000, 4000, 5000, 6000, 7000, 8000, or 10000 cis regulatory elements (e.g., enhancers).

The screening method can be used to screen cis regulatory elements for cell type-specific expression in any tissue, organ, organoid, virtual organ, or cells (e.g., a cell population comprising one or more cell types). The tissue, organ, organoid, or cells can be derived from a subject (e.g., an animal, mammal, primate, or human). In embodiments, the screening method can be used to screen cis regulatory elements for cell-specific expression in a community of cells comprising about or at least about 2, 3, 4, 5, 10, 20, 30, 40, 50, 75, 100, 200, 300, 400, 500, 1000, 10000, or more cell types. In some embodiments, the tissue or organ forms part of the central nervous system. Non-limiting examples of organs, tissues, or cell types include bone marrow, cardiac neurons, eye neurons, ear neurons, heart cells, immune cells, kidney cells, liver cells, the retina, a kidney, the brain, the cortex, the cerebellum, the gut, motor neurons, pain neurons, parvalbumin (PV) interneurons, peripheral neurons, proprioceptive neurons, somatostatin (SST) expressing neurons, sympathetic neurons, and vesicular glutamate transport (Vglut) neurons.

In embodiments, the screening method can be used to screen cis regulatory elements for cell type-specific expression during various developmental stages of a cell, cell community, organoid, or subject, and/or during various disease states (e.g., inflammation).

Further non-limiting examples of cell types include Exocrine secretory epithelial cells (e.g., Brunner's gland cells in duodenum, Insulated goblet cells of respiratory and digestive tracts, Stomach cells (e.g., Foveolar cells, Chief cells, and Parietal cells), Pancreatic acinar cells, Paneth cells of small intestine, Type II pneumocyte of lung, and Club cells of lung), Barrier cells (e.g., Type I pneumocytes (lung), Gall bladder epithelial cells, Centroacinar cells (pancreas), Intercalated duct cells (pancreas), and Intestinal brush border cells (with microvilli)), Hormone-secreting cells (e.g., Enteroendocrine cells (e.g., K cells, L cells, I cells, G cells, Enterochromaffin cells, Enterochromaffin-like cells, N cells, S cells, D cells, and Mo cells (or M cell)), Thyroid gland cells (e.g., Thyroid epithelial cells, Parafollicular cells), Parathyroid gland cells (e.g., Parathyroid chief cells, and Oxyphil cells), Pancreatic islets (islets of Langerhans) (e.g., Alpha cells, Beta cells, Delta cells, Epsilon cells, and PP cells (gamma cells)), Exocrine secretory epithelial cells (e.g., Salivary gland mucous cells, Salivary gland serous cells, Von Ebner's gland cells in tongue, Mammary gland cells, Lacrimal gland cells, Ceruminous gland cells in ear, Eccrine sweat gland dark cells, Eccrine sweat gland clear cells, Apocrine sweat gland cells, Gland of Moll cells in eyelid, Sebaceous gland cells, and Bowman's gland cells in nose), Hormone-secreting cells (e.g., Anterior/Intermediate pituitary cells, (e.g., Corticotropes, Gonadotropes, Lactotropes, Melanotropes, Somatotropes, and Thyrotropes), Magnocellular neurosecretory cells, secrete oxytocin and vasopressin, Parvocellular neurosecretory cells, and Chromaffin cells (adrenal gland)), Epithelial cells (e.g., Keratinocytes (differentiating epidermal cell), Epidermal basal cells (stem cell). Melanocytes, Trichocytes (e.g., Medullary hair shaft cells, Cortical hair shaft cells, Cuticular hair shaft cells, Huxley's layer hair root sheath cells, Henle's layer hair root sheath cells, and Outer root sheath hair cells), Surface epithelial cells (e.g., of cornea, tongue, mouth, nasal cavity, distal anal canal, distal urethra, and distal vagina), basal cells (stem cells) (e.g., of cornea, tongue, mouth, nasal cavity, distal anal canal, distal urethra, and distal vagina), Intercalated duct cells (salivary glands), Striated duct cells (salivary glands), Lactiferous duct cells (mammary glands), and Ameloblasts), Oral cells (e.g., Odontoblasts, and Cementoblasts), Nervous system cells (e.g., Sensory transducer cells (e.g., Auditory inner hair cells of organ of Corti, Auditory outer hair cells of organ of Corti, Basal cells of olfactory epithelium (stem cell for olfactory neurons), Cold-sensitive primary sensory neurons, Heat-sensitive primary sensory neurons, Merkel cells of epidermis, Olfactory receptor neurons, Pain-sensitive primary sensory neurons, Photoreceptor cells of retina in eye (e.g., Photoreceptor rod cells, Photoreceptor blue-sensitive cone cells of eye, Photoreceptor green-sensitive cone cells of eye, and Photoreceptor red-sensitive cone cells of eye) Proprioceptive primary sensory neurons, Touch-sensitive primary sensory neurons, Chemoreceptor glomus cells of carotid body cell (blood pH sensor), Outer hair cells of vestibular system of ear (acceleration and gravity), Inner hair cells of vestibular system of ear (acceleration and gravity), and Taste receptor cells of taste bud), Autonomic neuron cells (e.g., Cholinergic neurons (various types), Adrenergic neural cells (various types), and Peptidergic neural cells (various types)), Sense organ and peripheral neuron supporting cells (e.g., Inner pillar cells of organ of Corti, Outer pillar cells of organ of Corti, Inner phalangeal cells of organ of Corti, Outer phalangeal cells of organ of Corti, Border cells of organ of Corti, Hensen's cells of organ of Corti, Vestibular apparatus supporting cells, Taste bud supporting cells, Olfactory epithelium supporting cells, Olfactory ensheathing cells, Schwann cells, and Satellite glial cells, Enteric glial cells), Central nervous system neurons and glial cells (e.g., Neuron cells (e.g., Interneurons (e.g., Basket cells, Cartwheel cells, Stellate cells, Golgi cells, Granule cells, Lugaro cells, Unipolar brush cells, Martinotti cells, Chandelier cells, Cajal-Retzius cells, Double-bouquet cells, Neurogliaform cells, Retina horizontal cells, Amacrine cells (e.g., Starburst amacrine cells), and Spinal interneurons (e.g., Renshaw cells)), and Principal cells (e.g., Spindle neurons, Fork neurons, Pyramidal cells (e.g., Place cells, Grid cells, Speed cells, Head direction cells, and Betz cells), Stellate cells (e.g., Boundary cells), Bushy cells, Purkinje cells, and Medium spiny neurons)), Astrocytes, Oligodendrocytes, Ependymal cells (e.g., Tanycytes), and Pituicytes), Lens cells (e.g., Anterior lens epithelial cells and Crystallin-containing lens fiber cells), Central nervous system neurons or glial cells, Cells derived primarily from mesoderm, Metabolism and storage cells (e.g., Adipocytes, such as White fat cells or Brown fat cells, and Liver lipocytes), Secretory cells (e.g., Cells of the Adrenal cortex (e.g., Cells of the Zona glomerulosa produce mineralocorticoids, Cells of the Zona fasciculata produce glucocorticoids, and Cells of the Zona reticularis produce androgens), Theca interna cell of ovarian follicle secreting estrogen, Corpus luteum cell of ruptured ovarian follicle secreting progesterone (e.g., Granulosa lutein cells and Theca lutein cells), Leydig cell of testes secreting testosterone, Seminal vesicle cell, Prostate gland cells, Bulbourethral gland cells, Bartholin's gland cells, Gland of Littre cells, Uterus endometrium cells (carbohydrate secretion), Juxtaglomerular cells, Macula densa cells of kidney, Peripolar cells of kidney, and Mesangial cell of kidney), Barrier cells, Urinary system cells (e.g. Parietal epithelial cells, Podocytes, Proximal tubule brush border cells, Loop of Henle thin segment cells, Kidney distal tubule cells, Kidney collecting duct cells (e.g., Principal cells and Intercalated cells), and Transitional epithelium cells (lining urinary bladder)), Reproductive system cells (e.g., Duct cells (of seminal vesicle, prostate gland, etc.), Efferent ducts cells, Epididymal principal cells, and Epididymal basal cells), Circulatory system cells (e.g., Endothelial cells), Extracellular matrix cells (e.g., Planum semilunatum epithelial cells of vestibular system of ear (proteoglycan secretion), Organ of Corti interdental epithelial cells (secreting tectorial membrane covering hair cells), Loose connective tissue fibroblasts, Corneal fibroblasts (corneal keratocytes), Tendon fibroblasts, Bone marrow reticular tissue fibroblasts, Other nonepithelial fibroblasts, Pericytes (e.g., Hepatic stellate cells (Ito cell)). Nucleus pulposus cells of intervertebral disc, Hyaline cartilage chondrocyte, Fibrocartilage chondrocyte, Elastic cartilage chondrocytes, Osteoblasts/osteocytes, Osteoprogenitor cell (stem cell of osteoblasts), Hyalocyte of vitreous body of eye, Stellate cells of perilymphatic space of ear, and Pancreatic stellate cells), Contractile cells (e.g., Skeletal muscle cells, Red skeletal muscle cells (slow twitch), White skeletal muscle cells (fast twitch), Intermediate skeletal muscle cells, Nuclear bag cells of muscle spindle, Nuclear chain cells of muscle spindle, and Myosatellite cells (stem cell)), Cardiac muscle cells (e.g., SA node cells and Purkinje fiber cells), Smooth muscle cells (various types)., Myoepithelial cells of iris, and Myoepithelial cells of exocrine glands), Blood and immune system cells (e.g., Erythrocytes and precursor erythroblasts, Megakaryocytes, Platelets, Monocytes, Connective tissue macrophage (various types), Epidermal Langerhans cell, Osteoclasts, Dendritic cells, Microglial cells, Neutrophil granulocytes and precursors (myeloblast, promyelocyte, myelocyte, metamyelocyte), Eosinophil granulocytes and precursors, Basophil granulocytes and precursors, Mast cells, Helper T cells, Regulatory T cells, Cytotoxic T cells, Natural killer T cells, B cells, Plasma cells, Natural killer cells, and Hematopoietic stem cells and committed progenitors for the blood and immune system (various types)), Germ cells (e.g., Oogonium/Oocytes, Spermatids, Spermatocytes, Spermatogonium cells (stem cell for spermatocyte), and Spermatozoon), Nurse cells (e.g., Granulosa cells, Sertoli cells, and Epithelial reticular cells), and Interstitial cells (e.g., Interstitial kidney cells). In embodiments, the cells are part of the cardiovascular system (e.g., heart and lungs), digestive system (e.g., salivary glands, esophagus, stomach, liver, gall bladder, pancreas, intestines, colon, rectum, and anus), endocrine system (e.g., hypothalamus, pituitary gland, pineal body or pineal gland, thyroid, parathyroids, and adrenals), excretory system (e.g., kidneys, ureters, bladder, and urethra), lymphatic system, integumentary system (e.g., skin, hair, and nails), muscular system, nervous system (e.g., brain, spinal cord, and nerves), reproductive system (e.g., sex organs such as ovaries, fallopian tubes, uterus, vulva, vagina, testes, vas deferens, seminal vesicles, prostate, and penis), and/or skeletal system (e.g., bones, cartilage, ligaments, and tendons).

In some cases, the cells are from the nervous system, brain, cerebrum, cerebral hemispheres, diencephalon, the brainstem, midbrain, pons, medulla oblongata, cerebellum, the spinal cord, the ventricular system, choroid plexus, peripheral nervous system, see also: list of nerves of the human body, nerves, cranial nerves, spinal nerves, ganglia, enteric nervous system, sensory organs, sensory system, eye, cornea, iris, ciliary body, lens, retina, ear, outer ear, earlobe, eardrum, middle ear, ossicles, inner ear, cochlea, vestibule of the ear, semicircular canals, olfactory epithelium, tongue, taste buds, integumentary system, mammary glands, skin, subcutaneous tissue, immune system, muscular system, musculoskeletal system, bone, human skeleton, joints, ligaments, muscular system, tendons, digestive system, mouth, teeth, tongue, salivary glands, parotid glands, submandibular glands, sublingual glands, pharynx, esophagus, stomach, small intestine, duodenum, jejunum, ileum, large intestine, liver, gallbladder, mesentery, pancreas, anal canal and anus, blood cells, respiratory system, nasal cavity, pharynx, larynx, trachea, bronchi, lungs, diaphragm, urinary system, kidneys, ureter, bladder, urethra, reproductive organs, female reproductive system, internal reproductive organs, ovaries, fallopian tubes, uterus, vagina, external reproductive organs, vulva, clitoris, placenta, male reproductive system, internal reproductive organs, testes, epididymis, vas deferens, seminal vesicles, prostate, bulbourethral glands, external reproductive organs, penis, scrotum, endocrine system, pituitary gland, pineal gland, thyroid gland, parathyroid glands, adrenal glands, pancreas, circulatory system, heart, patent foramen ovale, arteries, veins, capillaries, lymphatic system, lymphatic vessel, lymph node, bone marrow, thymus, spleen, gut-associated lymphoid tissue, tonsils, or interstitium. Further non-limiting examples of cell types include those described in PCT/US2019/064616, the disclosure of which is incorporated herein by reference in its entirety for all purposes. Neurons are polarized cells with defined regions consisting of the cell body, an axon, and dendrites, although some types of neurons lack axons or dendrites. Their purpose is to receive, conduct, and transmit impulses in the nervous system. Neurons can be classified a number of different ways: anatomical, physiological, and developmental. Anatomical classes are defined first by the location of the neuron in the nervous system. Neurons are further distinguished from each other by features which include dendritic and axon morphology. Anatomical features also include synaptic connectivity (e.g., inputs and outputs) and molecular phenotype (e.g., the particular neurotransmitters, receptors, and ion channels expressed by a neuron). Neurons can be classified by their physiological properties. This includes their general function (e.g., sensory, motor, interneuron). Functions can also include whether the neuron is a relay neuron or a local interneuron or whether it is involved in sensory processing or correction of motor responses. Physiological actions can also include the firing properties of the neuron (e.g., bursting, tonic, quiescent). Developmental classifications of neurons are based upon the lineage that the cell derives from. The number of neurons in a particular class can vary over orders of magnitude from individual neurons in some classes to millions of neurons in other classes.

In some instances, the cells are located in a specific layer or layers of the cerebral cortex, for example layer(s) I, II, III, IV, V, and/or VI. Layer I is the molecular layer, which contains very few neurons; layer II is the external granular layer; layer III is the external pyramidal layer; layer IV is the internal granular layer; layer V is the internal pyramidal layer; and layer VI is the multiform, or fusiform layer. In some embodiments of any of the aspects, the at least one GRE exhibits cell-type specificity for cells (e.g., SST interneurons) in layer IV and V of the cerebral cortex. Non-limiting examples of cells include cerebral cortex cells, such as pyramidal neurons; glial cells; Cajal-Retzius cells; subpial granular layer cells; spiny stellate cells; small pyramidal neurons; stellate neurons; medium-size pyramidal neurons; non-pyramidal neurons (e.g., with vertically oriented intracortical axons); large pyramidal neurons; giant pyramidal cells (e.g., Betz cells); small spindle-like pyramidal neurons; and multiform neurons; or GABAergic rosehip neurons. In embodiments, the neuron is an excitatory or inhibitory neuron, such as a glutamatergic excitator neuron cell type. In some embodiments, the cell is a neuron that produces a specific neurotransmitter, including but not limited to arginine, aspartate, glutamate, gamma-aminobutyric acid, glycine, D-serine, acetylcholine, dopamine, norepinephrine (noradrenaline), epinephrine (adrenaline), serotonin (5-hydroxytryptamine), histamine, phenethylamine, N-methylphenethylamine, tyramine, octopamine, synephrine, tryptamine, N-methyltryptamine, anandamide, 2-arachidonoylglycerol, 2-arachidonyl glyceryl ether, N-arachidonoyl dopamine, virodhamine, adenosine, adenosine triphosphate, or nicotinamide adenine dinucleotide. In some cases, the neuron produces a specific neuropeptide, including but not limited to Bradykinin, Corticotropin releasing hormone, Urocortin, Galanin, Galanin-like peptide, Gastrin, Cholecystokinin, Neuropeptide Y, Pancreatic polypeptide, Peptide YY, Enkephalin, Dynorphin, Endorphin, Endomorphin, Nociceptin/orphanin FQ, Orexin A, Orexin B, Kisspeptin, Neuropeptide FF, Prolactin-releasing peptide, Pyroglutamylated RFamide peptide, Secretin, Motilin, Glucagon, Glucagon-like peptide-1, Glucagon-like peptide-2, Vasoactive intestinal peptide, Growth hormone-releasing hormone, Pituitary adenylate cyclase-activating peptide, Somatostatin, Neurokinin A, Neurokinin B, Substance P, Neuropeptide K, Agouti-related peptide, N-Acetylaspartylglutamate, Cocaine- and amphetamine-regulated transcript, Bombesin, Gastrin releasing peptide, Gonadotropin-releasing hormone, or Melanin-concentrating hormone. In some instances, the neuron produces a specific gasotransmitter (i.e., a gaseous signaling molecule), including but not limited to Nitric oxide, Carbon monoxide, or Hydrogen sulfide.

The screening vectors and screening methods are applicable across brain regions or with specific cell populations defined by connectivity (e.g., anterograde or retrograde Cre delivery), and can be adapted in embodiments to screen for various transcriptional or post-transcriptional regulatory elements. Further, the screening vectors allow for scaling the tissue sampling to read out activity in rare cell populations. The ability to assess expression in rare cell populations is also enabled by the use of unique barcode readouts. By assessing the distribution of expression strength (NGS reads) from individual barcodes (many of which represent expression from single cells), it is possible to detect expression that might be missed by bulk assessments.

Not intending to be bound by theory, when multiple screening vectors containing screening vectors are introduced to a cell, the screening vectors concatamerize. This concatamerization leads to cross-talk between the different enhancers. In embodiments, these concatemers are eliminated or reduced in a cell when a recombinase (e.g., FLP/flippase) contacts the first set of recombinase recognition sites (e.g., FRT3 sequences) and produces mini-circles (see FIG. 6). Typically, mini-circle formation does not cause any recombination between an enhancer and a barcode and/or invertible spacer. Mini-circle formation can eliminate crosstalk or interference between cis regulatory elements coadministered to a cell using the screening vectors. In embodiments, cells can express high levels of transcripts from the screening vectors and driven by a cis regulatory element only after minicircle formation. In embodiments, the method provides for improved signal-to-noise relative to a method that does not involve mini-circle formation. Detection of cDNAs can be performed using mini-circle specific PCR primers.

Further, in embodiments, when the mini-circles are formed, the 3′UTR is properly positioned to stabilize mRNA transcribed from the screening vector and/or an mRNA destabilizing element included in mRNA transcribed from the screening vectors in their initial configuration is removed and not included in the mRNA transcribed from the mini-circles.

In embodiments, when the screening vector is introduced into a cell expressing an appropriate recombinase (e.g., Cre), contacting the screening vectors with the recombinase results in the inversion of the invertible spacer. When this inversion is detected in mRNA sequenced from cells infected by the screening vectors, it indicates that the cells expressing the recombinase are cells in which the enhancer has activity.

In embodiments, sensitivity of the screen is improved by selectively amplifying and sequencing only those barcodes associated with an inverted invertible spacer (floxed tag) and comparing them across Cre lines. Such increased sensitivity can allow for detection of the inverted spacers in rare populations. Another strategy for increasing screen sensitivity involves splitting the library into 2, 3, 4, 5, 6, 7, 8, 9, 10, 15, 20 or more smaller libraries. Smaller libraries can be more sensitive since each individual enhancer will represent a larger fraction of the library and will therefore be screened through more cells.

While not intending to be bound by theory, enhancers that show activity and an elevated rate of inversion as compared to WT animals in broadly expressing Cre populations could result from expression within and outside of the Cre target population or highly specific expression within a subpopulation of the broad Cre+ population. Advantageously, the screening method can distinguish between these profiles by assessing the specificity not only within animals, but also across Cre lines. Therefore, the screen can generate a detailed set of information that can be used to choose enhancers for individual characterization or for use in controlling expression of a gene of interest in a target cell population.

### Polynucleotide Sequencing

In particular embodiments, transcripts produced under the control of an enhancer are measured by a sequencing-based technique (e.g., next-generation sequencing). The sequencing allows for detection of inversions associated with a transcript as well as a barcode associated with the transcript.

Preparation of a library for sequencing may involve an amplification step. Amplification may involve thermocycling (e.g., PCR) or isothermal amplification (such as through the methods NEAR, RNA-Seq, RPA or LAMP). Amplification can refer to any method employing a primer and a polymerase capable of replicating a target sequence with reasonable fidelity. Amplification may be carried out by natural or recombinant DNA polymerases, such as TaqGold™, T7 DNA polymerase, Klenow fragment of E. coli DNA polymerase, and reverse transcriptase. A preferred amplification method is PCR In some embodiments, isolated RNA is contacted with a reverse transcriptase to produce cDNA for sequencing and/or PCR amplification.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illuminam sequencing), sequencing with mass spectrometry, and tunneling currents DNA sequencing.

### Hardware and Software

The present invention also provides a computer system useful in analyzing data associated with screening libraries of cis regulatory elements, analyzing sequence data, and/or characterizing cis regulatory element activities.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Compositions

Provided also are compositions for use in screening libraries of cis regulatory elements. In an embodiment, the composition includes an AAV vector or virus particle, such as one containing a screening vector, as described herein and an acceptable carrier, excipient, or diluent.

The screening vectors and/or AAV vectors may be contained in any appropriate amount in any suitable carrier substance, and is/are generally present in an amount of 0.01-95% by weight of the total weight of the composition. The composition may be provided in a form that is suitable for a parenteral (e.g., subcutaneous, intravenous, intramuscular, or intraperitoneal) administration route, such that the agent, such as a vector described herein, is systemically delivered. In an embodiment, systemic injection of an rAAV vector as described herein allows for the characterization of specificity of expression associated with cis regulatory elements across brain regions, organs, or tissues. In some instances, a reporter product is also encoded by the vector. The compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Compositions may be formulated to release the vectors substantially immediately upon administration or at any predetermined time or time after administration. The latter types of compositions are generally known as controlled release formulations, which include (i) compositions that create a substantially constant concentration of the agent within the body over an extended period of time; (ii) compositions that after a predetermined lag time create a substantially constant concentration of the drug within the body over an extended period of time; (iii) compositions that sustain action during a predetermined time period by maintaining a relatively constant, effective level in the body with concomitant minimization of undesirable side effects associated with fluctuations in the plasma level of the active substance (sawtooth kinetic pattern); (iv) compositions that localize action by, e.g., spatial placement of a controlled release composition adjacent to or in contact with a target site or location, e.g., in a region of a tissue or organ; (v) compositions that allow for convenient dosing, such that doses are administered, for example, once every one, two, or several weeks; and (vi) compositions that target a specific tissue or cell type using carriers, chemical derivatives, or specifically designed vectors (e.g., comprising a certain capsid composition) to deliver the vector.

The composition may be administered systemically, for example, in an acceptable buffer such as physiological saline. In an embodiment, systemic injection of an rAAV vector as described herein allows for the characterization of specificity of expression associated with enhancers across brain regions, tissues, the central nervous system, or an organ(s).

Routes of administration include, for example, intracranial, parenteral, subcutaneous (s.c.), intravenous (i.v.), intraperitoneal (i.p.), intramuscular (i.m.), or intradermal administration. The amount of the vector to be administered can vary depending upon the requirements of a given screen. Generally, amounts will be in the range of those used for other viral vector-based agents employed in the delivery of polynucleotides to cells. In embodiments, about, at least about, and/or no more than about 1×10e5, 1×10e6, 1×10e7, 1×10e8, 1×10e9, 1×10e10, 1×10e11, 1×10e12, 1×10e13, 1×10e14, or 1×10e15 vector genomes are delivered to a subject (e.g., a mouse) to screen a library of enhancers. A composition is administered at a level that is effective in meeting the objectives of a screen.

The composition may be in the form of a solution, a suspension, an emulsion, an infusion device, or a delivery device for implantation, or it may be presented as a dry powder to be reconstituted with water or another suitable vehicle before use. Apart from the screening vector, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising screening vectors is formulated for intravenous delivery. As noted above, the compositions according to the described embodiments may be in a form suitable for sterile injection. To prepare such a composition, the suitable therapeutic(s) are dissolved or suspended in a parenterally acceptable liquid vehicle. Acceptable vehicles and solvents that may be employed include water, water adjusted to a suitable pH by addition of an appropriate amount of hydrochloric acid, sodium hydroxide or a suitable buffer, 1,3-butanediol, Ringer's solution, isotonic sodium chloride solution and dextrose solution. The aqueous formulation may also contain one or more preservatives (e.g., methyl, ethyl, or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Kits

Also provided are kits for screening cis regulatory for cell type-specific expression in vivo. In one embodiment, the kit provides a composition containing an effective amount of screening vectors or viral particles as described herein, optionally containing a library of cis regulatory elements (e.g., enhancers) to be screened. In some embodiments, the kit provides screening vectors suitable for preparation of libraries of cis regulatory elements to be screened.

In some embodiments, the kit comprises a sterile container which contains the composition; such containers can be boxes, ampoules, bottles, vials, tubes, bags, pouches, blister-packs, or other suitable container forms known in the art. The containers can be made of plastic, glass, laminated paper, metal foil, or other materials suitable for holding medicaments.

The kit can include instructions for use of the screening vectors to screen cis regulatory elements and/or to prepare libraries of cis regulatory elements to be screened. In embodiments, the instructions describe how to analyze data produced from a screen undertaken using the screening vectors. The instructions may be printed directly on the container (when present), or as a label applied to the container, or as a separate sheet, pamphlet, card, computer-readable medium, or folder supplied in or with the container.

The practice of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Development of a Vector for Large-Scale Screening to Identify Cell Type-Specific Enhancers in Mice

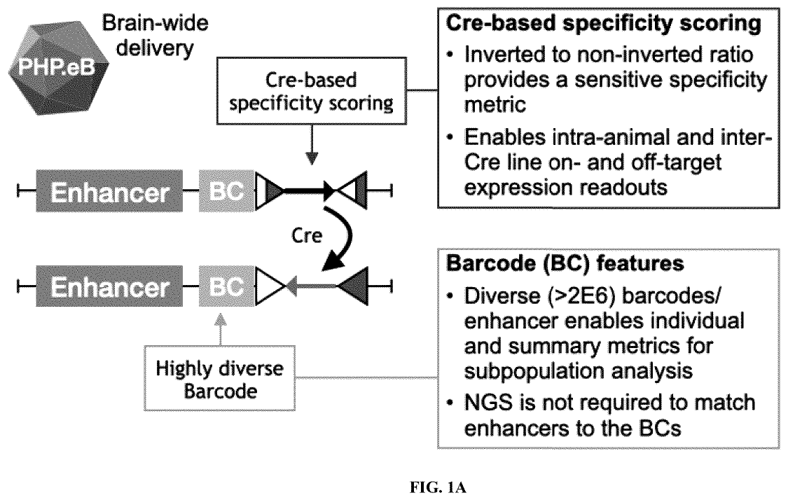

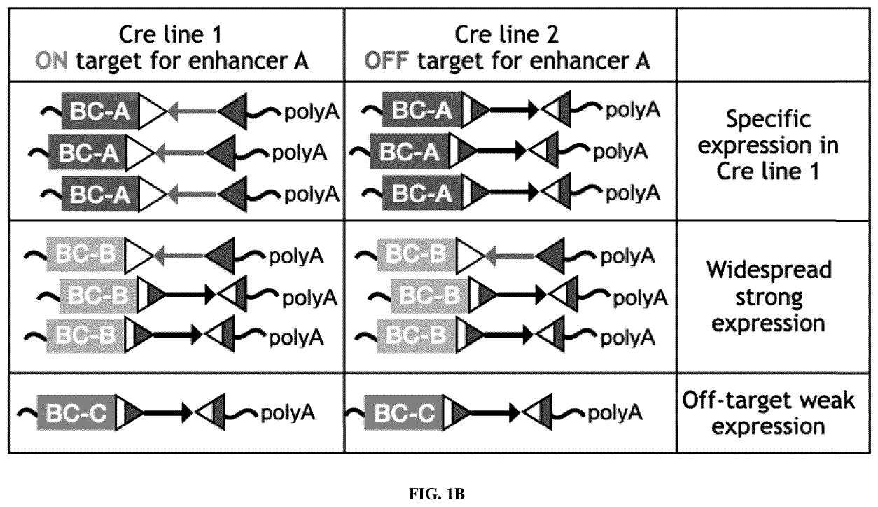

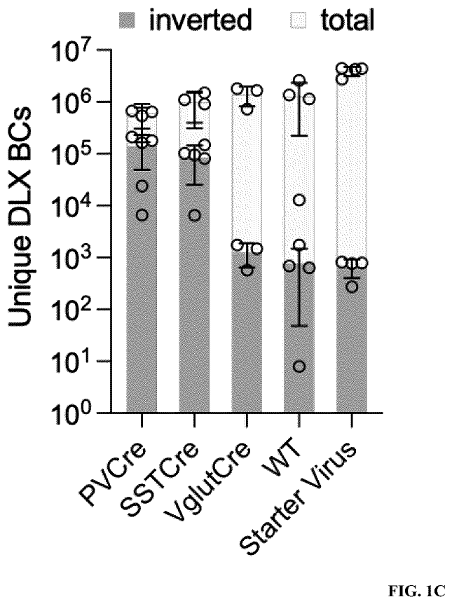

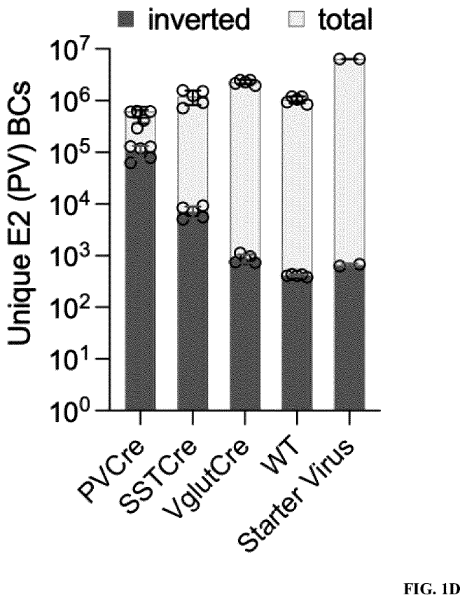

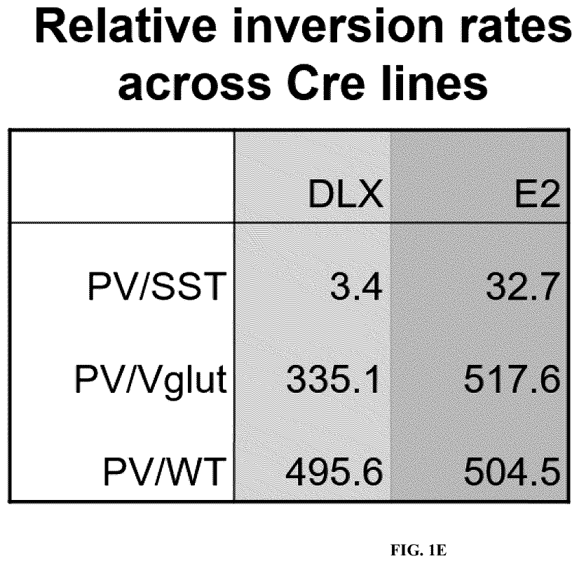

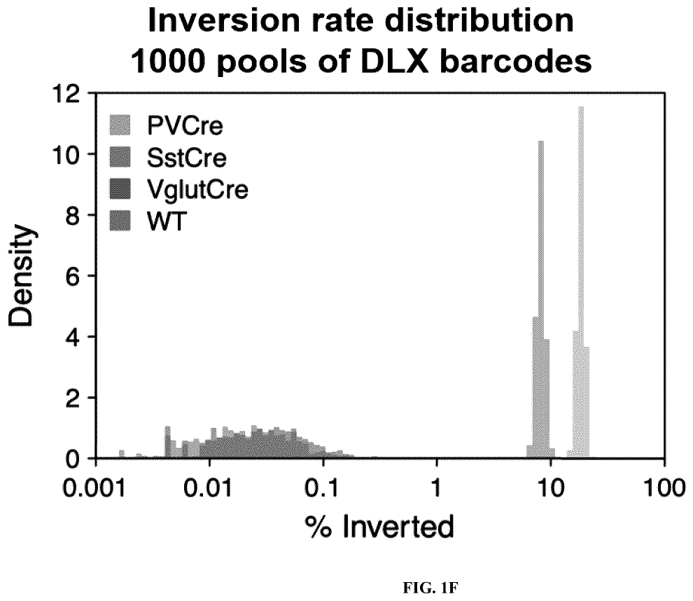

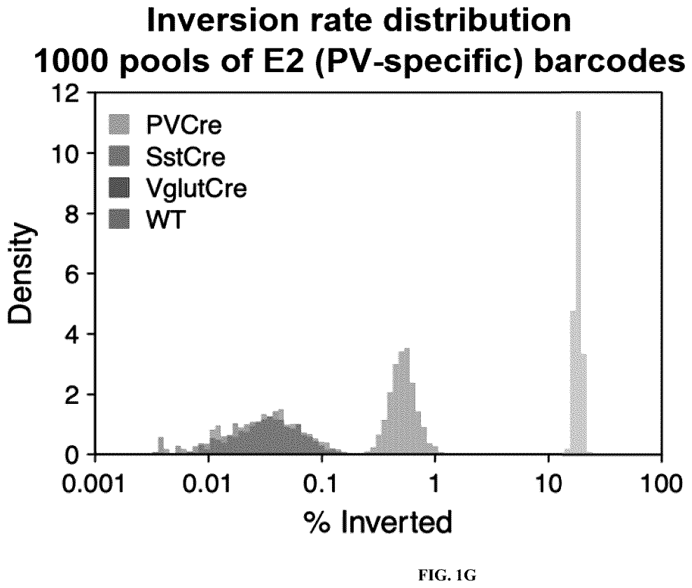

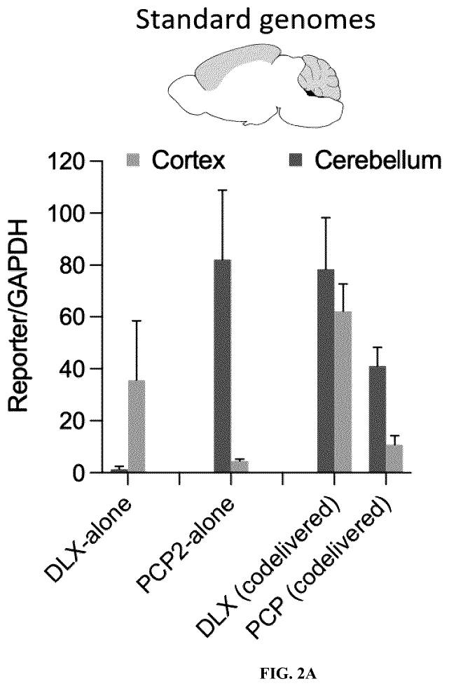

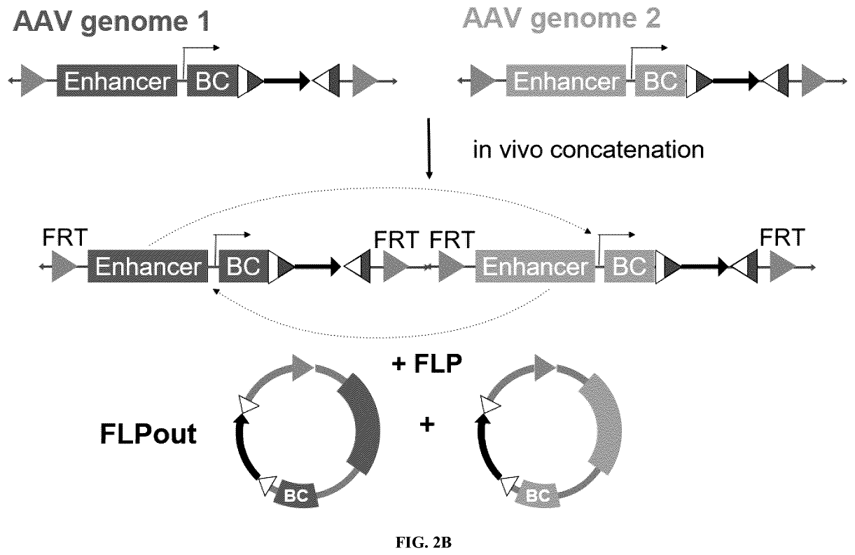

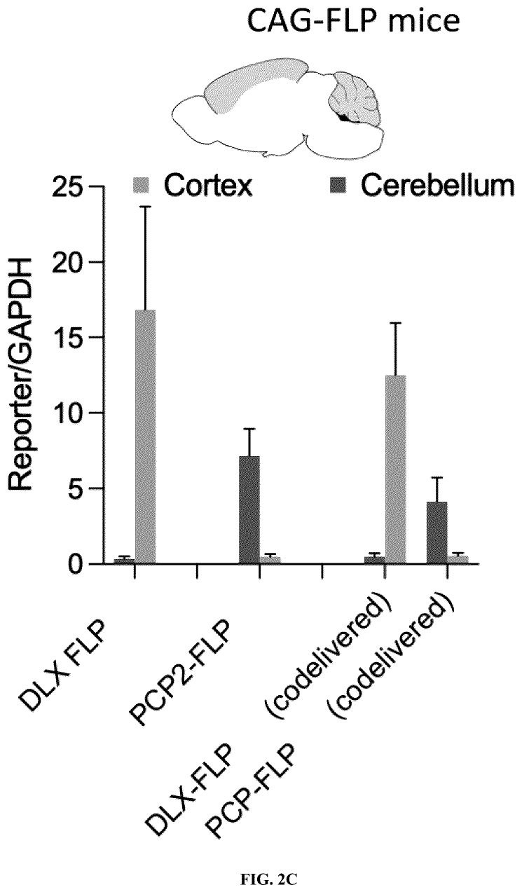

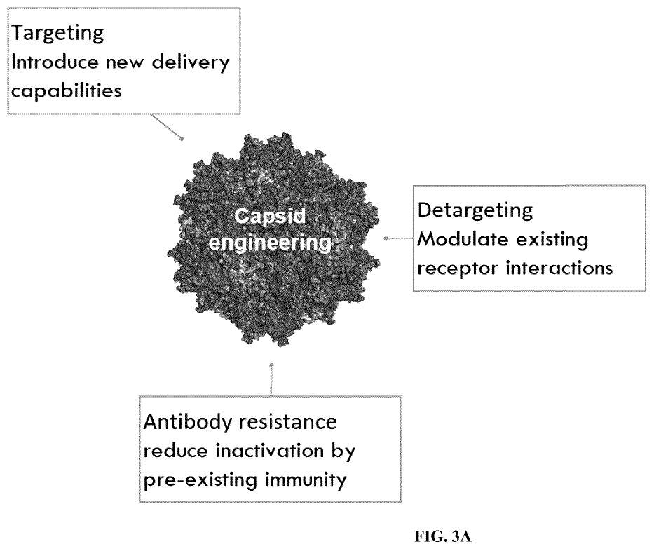

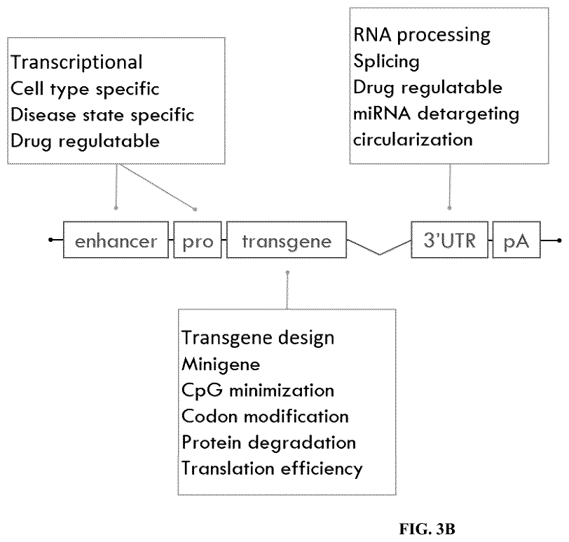

Experiments were undertaken to develop a high-throughput enhancer screening vector and method (FIGS. 2B, 4-7, 11, 14A, and 15) to evaluate the expression and specificity of hundreds to thousands of cis regulatory elements simultaneously in adult mice (FIGS. 1A-1G and 2A-2C). In various embodiments, the screening platform can be used to identify cis regulatory elements (e.g., enhancers) that may be used to control the expression of a gene delivered to a cell using an AAV vector (see, e.g., FIGS. 3A and 3B). For example, in some instances, a capsid that broadly delivers genes throughout the central nervous system (CNS) is combined with specific regulatory elements to restrict expression to the target cell types.

The screening vector and method developed included several key features. First, the vector, which is an adeno-associated virus (AAV) vector, includes a highly reproducible and quantitative readout. The vector links each enhancer to millions of unique, highly diverse barcodes (FIG. 9) to enable quantitative readouts of activity. The barcodes enables single transduction level readouts of expression and specificity scores for each enhancer in a mixed library. The barcode design allowed for over 2.6 million unique and readable barcodes to be associated with each enhancer. Next generation sequencing was not required to match the barcodes with the enhancers.

Second, the vector allowed for specificity assessments from bulk RNA. Single-cell isolation was not required. The vector leveraged the Cre-lox system (a Cre-invertible element with mutant lox sites) to read out enhancer specificity in any cell type that can be made to selectively express Cre without the limitations associated with isolating single cells. The Cre-lox system allowed for tagging mRNA transcripts expressed in target cell populations expressing Cre. The system also enabled the scoring of the specificity of an enhancer for on- and off-target cell types by measuring the inversion ratio in individual samples as well as between samples with Cre expressed in different cell types, all from bulk RNA samples.

Third, the vector produced a high signal-to-noise readout by minimizing enhancer crosstalk that can occur as a result of AAV genome concatemerization. This was done using recombinase-mediated mini-circle formation, which increased signal-to-noise by separating individual enhancer-reporter-BC sequences. The vector minimized RNA stability from the AAV genome in the initial state (i.e., not as minicircles) or as concatemers by including an RNA degradation signal transcribed from the vectors in their initial state or as concatemers but not from minicircles formed following recombination. The vector located the WPRE and poly adenylation sequences upstream of the enhancer in the initial state or concatenated state to further destabilize transcripts formed prior to mini-circle formation, where mini-circle formation placed the elements into position to function properly in the 3′UTR of a transcript produced under the control of an enhancer.

Finally, the vector enabled brain-wide assessment of enhancer specificity through the use of systemic AAV-PHP.eB administration to deliver the enhancer library to the majority of neurons throughout the central nervous system (CNS).

Therefore, the screening vector addressed the following design parameters: 1) evaluation in context: screening in vivo using IV AAV-PHP.eB; 2) Specificity assessment: highly diverse barcode with near single-cell specificity readout allowed for individual read out of on and off target expression and specificity from individual cells; 3) Quantitative expression assessment even in rare cell types: measurements were taken using efficient bulk RNA recovery scalable to millions of cells allowing for high detection efficiency and quantitative readouts of activity and specificity even in rare cell populations; and 4) High signal-to-noise: individual AAV genomes were isolated allowing for the various genomes to act independent of one another.

A barcoding strategy (FIG. 9) was developed to allow for quantitative assessment of the transcriptional activity of individual enhancers within a library of hundreds to thousands of co-administered AAVs. The screening vectors were constructed so that each enhancer was coupled to its own highly diverse set of barcodes (>2 million unique barcodes per enhancer; minimum Hamming distance of 3 vs. any other barcode set) (FIG. 1A). Because the barcodes were predetermined (i.e., decodable/readable), next-generation sequencing of the virus library was not necessary in order to develop an enhancer-barcode look-up table. The diverse barcoding strategy dramatically improved data quality and reduced bias. The barcoding strategy enabled quantitative assessments of the number of (1) reads per enhancer, (3) unique barcodes, (2) reads per individual barcode, and the distribution of reads per individual barcodes.

The screening vectors allowed for determination of enhancer specificity using a Cre-lox strategy (FIGS. 1A and 1B). The vectors were designed to allow for an enhancer library encoded by the screening vectors to be delivered systemically to a panel of transgenic mice expressing Cre in each target cell population. The vectors contained a floxed, Cre-invertible tag adjacent to the barcode to enable expression assessments in both Cre⁺ and Cre⁻ cells within each sample using next-generation sequencing (NGS). The design of the vectors allowed for specificity (on- and off-target expression) to be scored through both intra-animal measurements (the ratio of inverted vs total enhancer barcodes in each sample) and across Cre lines (the relative inversion ratio for each enhancer across mice).

An overview of the design of the vector is provided in FIG. 5.

### Example 2: The Screening Vectors Allowed for Quantitative Scoring of the Expression and Specificity of Enhancers in Cre Populations

Experiments were undertaken to evaluate the ability of the screening vectors to facilitate specific quantitative scoring of transcript expression associated with enhancers in various Cre populations.

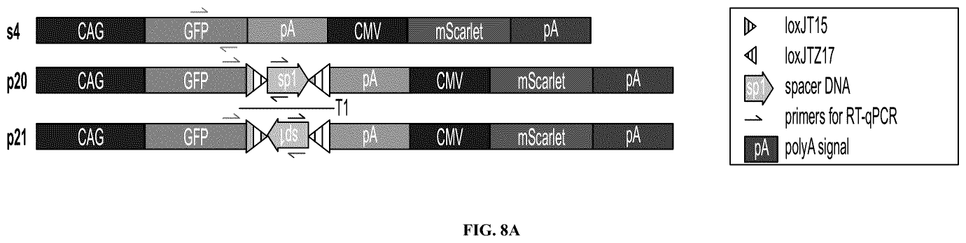

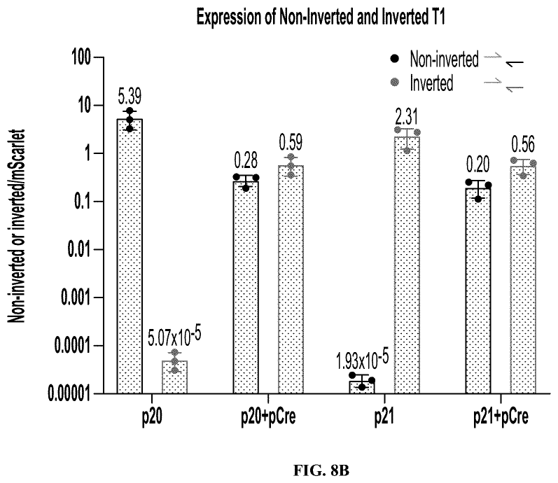

First, experiments were undertaken to determine whether the orientation of the invertible spacer sequence of the screening vectors introduced any bias (FIGS. 8A and 8B). It was determined that the orientation of the invertible spacers did not introduce any bias (FIG. 8B).

Next, an experiment was undertaken to assess the Cre-based specificity scoring both within a Cre-line and across animals using two well characterized enhancers. The experiment was designed to assess whether or not the screening vectors were capable of differentiating the specificity of the two enhancers by evaluating them in several no and off target cell types. The two well-characterized enhancers were DLX, which is broadly interneuron specific being expressed across all interneurons, and E2, which is specific to PV interneurons (PvIN), in three Cre lines and WT mice using a mix of 5 barcode sets per enhancer (more than 13M total possible barcodes for each enhancer) (FIGS. 1C-1G). The vectors containing DLX were expected to have high inversion rates in the two interneuron-specific Cre lines PV-Cre and SST-Cre and the vectors containing the E2 enhancer were expected to have high inversion rates in PV-Cre lines. The data confirmed that the vectors were able to provide specificity scores that match individual enhancer characterization data. More than 4×10e6 unique barcodes were detected in each vector library, and 1×10e5 to over 1×10e6 uniquely barcoded mRNAs from the brains of WT or Cre-expressing mice, indicating successful library production, successful vector delivery, and successful detection of barcoded library transcriptions (FIGS. 1C and 1D).

Then, to assess the ability to use Cre-mediated inversion as a readout of specificity, the ratio of Cre-tagged barcodes to total barcodes for both enhancers in three Cre lines and in WT mice was measured. The inversion ratio of the DLX enhancer barcodes was more than 300-fold higher in both PV-Cre and SST-Cre mice than in Vglut2-Cre (glutamatergic neuron-specific) or WT (Cre) mice, while the E2 enhancer was more than 500-fold higher in PV-Cre than Vglut2-Cre and more than 30-fold higher in PV-Cre than SST-Cre (FIG. 1E). These results were consistent with expectations based on the individually characterized activity of these enhancers, and convincingly demonstrated that the barcoding and Cre-based enhancer specificity readouts were quantitative and performed as expected.

To simulate a library of thousands of enhancers using these data, individual enhancer barcodes were randomly assigned to one of 1000 pools and then the mean specificity score (inversion rate) was assessed for each pool individually. The experiment simulated a 1000 enhancer library experiment. Individual enhancer barcodes were computationally pooled into 1000 defined subsets and then the specificity scores for each subset was individually determined. The distribution of the inversion rate for each enhancer in each Cre line was highly consistent across the 1000 pools, showing a tight distribution in on-target Cre lines (FIGS. 1F and 1G), showing that the system provided reliable, quantitative data in a library format.

Assessing the DLX enhancer in the Cre-based specificity screening using the screening vectors indicated that Cre inversion was detected in on-target cells (PV- and SST-Cre) at a rate that was 300 to 600 times higher than in off-target cells (Vglut1-Cre) (FIG. 1F). The assay was also sensitive to the lower specificity of the PV-specific E2 enhancer in PV cells vs SST cells (30 times higher in PV cells), which, while not intending to be bound by theory, likely resulted from low level expression from the E2 enhancer in a subset of SST cells (FIG. 1G). Indeed, the analysis showed that E2 associated barcodes were inverted at a lower but detectable rate in the SST-Cre mice, consistent with individual validation data (FIG. 1G). Expression was detected from 1×10e5 to over 1×10e6 unique barcodes across the three Cre lines and WT mice.

The above data demonstrate that the vectors can be used in methods to quantitatively score the expression and specificity of enhancers in Cre expressing populations. These data further demonstrate that the screening vectors facilitate gathering of quantitative data in a library format, and that the use of this approach to quantitatively score the expression and specificity of enhancers in each Cre population.

Critically, these results suggest that the Cre-based specificity scoring was sensitive enough to detect enhancers that are specific to subpopulations of cells within a broader class of cells defined by Cre expression. These results demonstrate that the screening vectors can be used to detect expression from hundreds of enhancers and assess their specificity.

### Example 3: The Screening Vectors Reduced Cross-Talk Between Screening Vectors Delivered to the Same Cell and Containing Different Enhancers

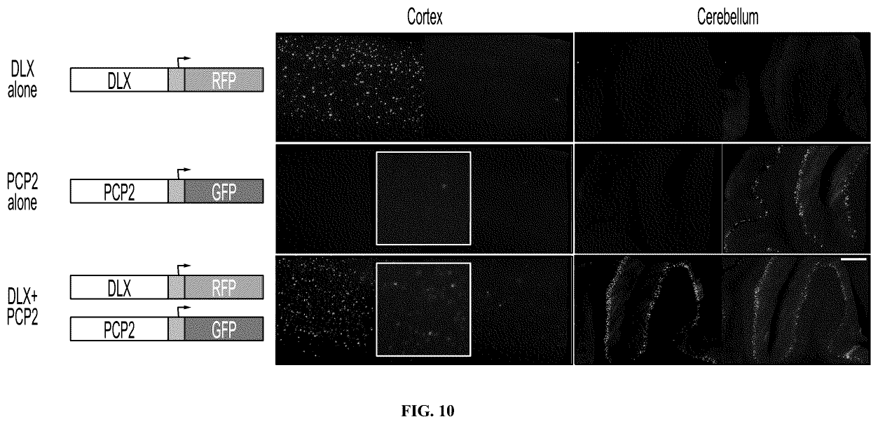

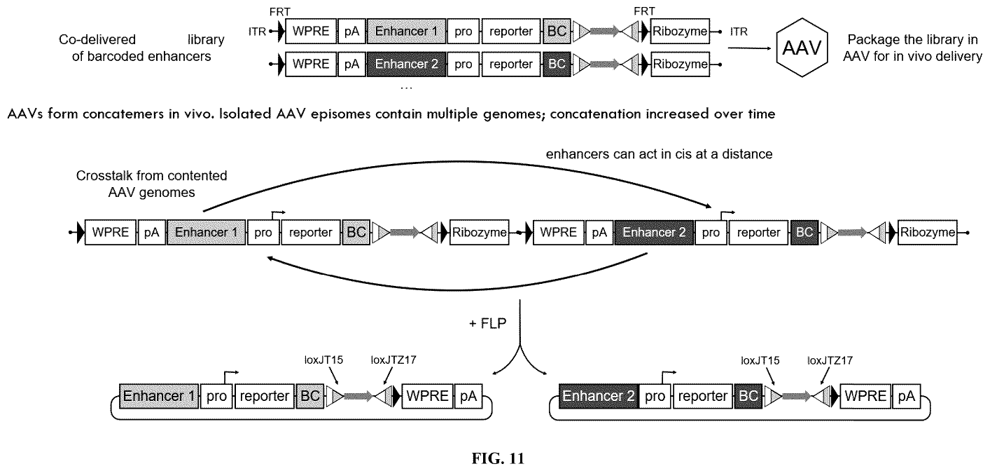

When AAV genomes are co-delivered to the same animal, there can be crosstalk between the genomes. For example, when an AAV genome containing a DLX-driven reporter was co-administered with an AAV genome containing a Purkinje cell-specific regulatory element (PCP2) using AAV-PHP.eB, there was interference (or crosstalk) between the genomes that caused unexpected expression of the DLX-driven reporter in Purkinje cells, and expression of the PCP2-driven reporter in cortical inhibitory neurons (FIGS. 2A and 10). Not intending to be bound by theory, it was hypothesize that the crosstalk occurred because AAV genomes that co-transduce cells can concatamerize in vivo (FIG. 11). Once concatenated, enhancers on different genomes can act in cis and affect the transcription of the gene(s) expressed from other AAV genome within a concatemer. This crosstalk also occurred with AAVs directly administered to the CNS.

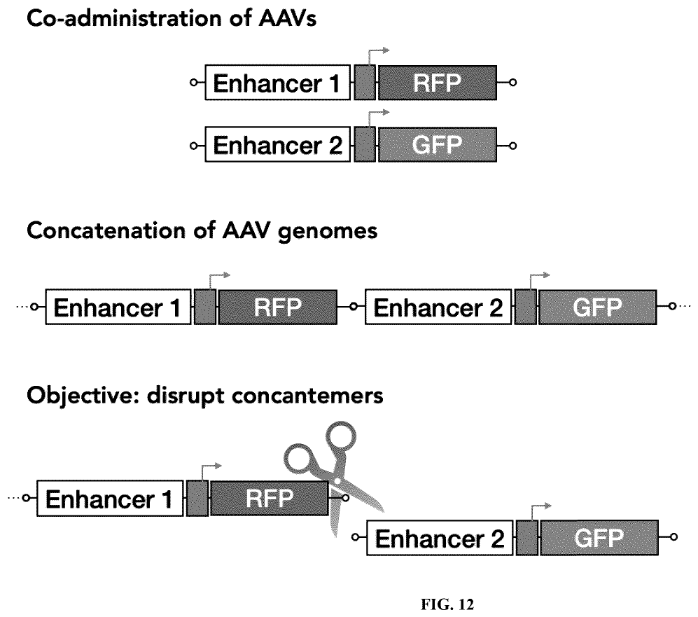

Therefore, the screening vectors were designed to include a system to minimize this crosstalk. The screening vectors were designed so that each individual AAV genome could be excised out from concatemers via Flp recombinase (flippase) to form individual DNA mini-circles (FIGS. 2B, 6, 11, 12, 14A, and 2B). To evaluate the ability of the vectors to reduce crosstalk between enhancers resulting from vector concatenation, DLX-reporter and PCP2-reporter vectors were delivered alone or co-delivered to ACTB-FLP mice using AAV-PHP.eB. Flp recombinase-based cutting of concatenated vectors (AAV genomes) to generate individual mini-circles reduced crosstalk/interference between enhancers delivered to the same cell(s) (FIGS. 2A and 2C). The vector design nearly eliminate crosstalk between DLX and PCP2 regulatory elements (FIG. 2C)

The vectors contained additional elements that strongly reduce expression in their initial or concatenated state (FIGS. 5, 13A, 13B, and 14A-14C), and the detection method was designed to enable selective detection of RNAs from minicircles. As described above, the vectors were designed so that a stabilizing 3′UTR would be added to transcripts produced under the control of the enhancer only following flippase recombination and mini-circle formation (FIGS. 5, 6, 11, and 15).

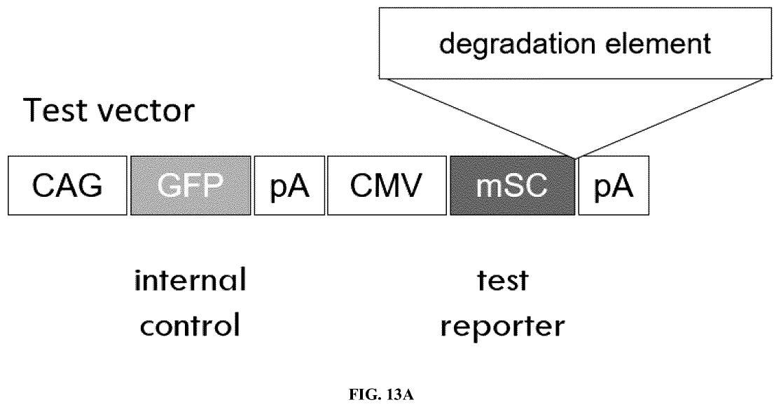

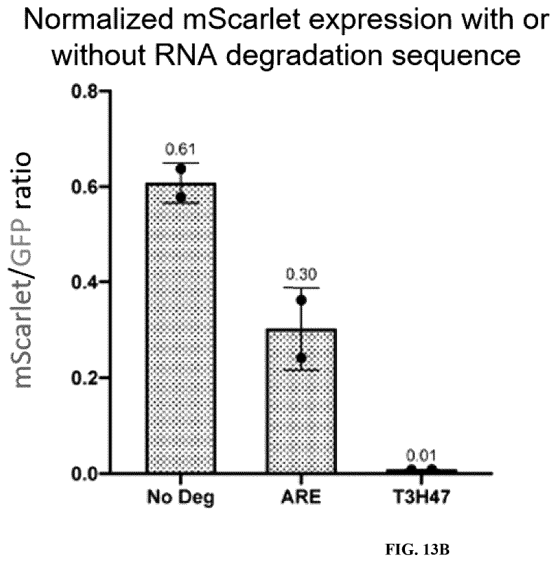

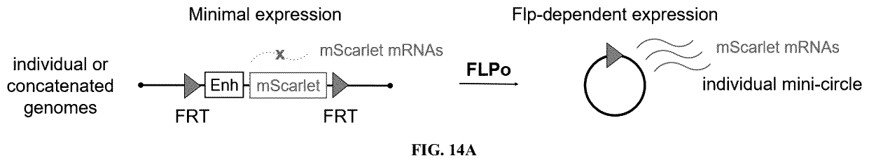

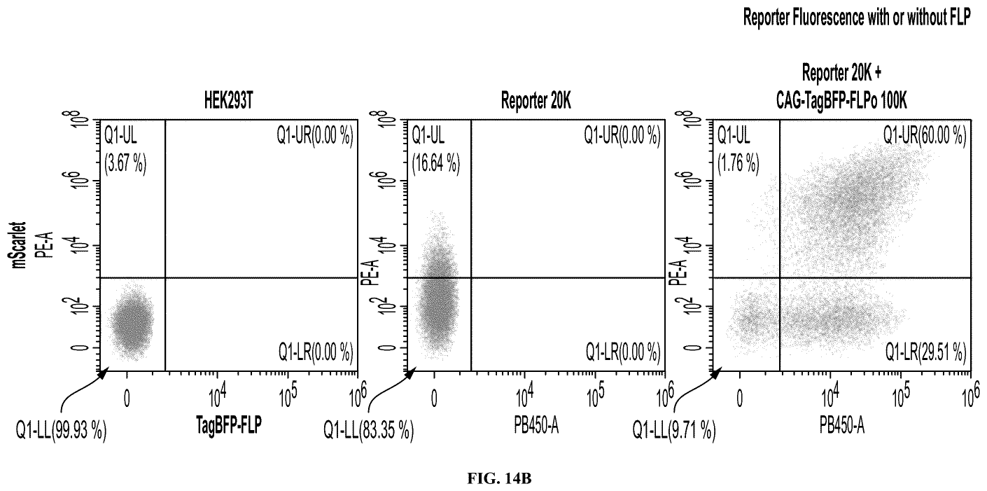

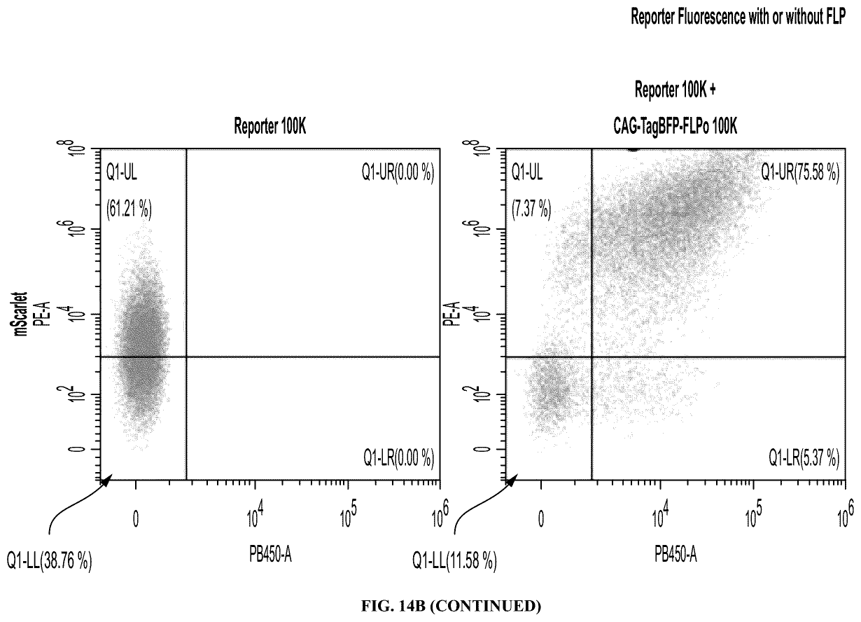

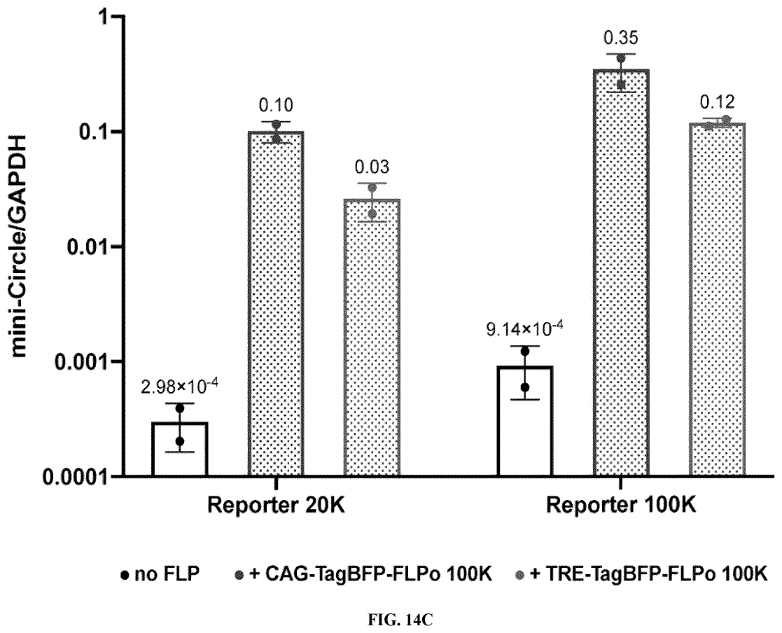

The vectors also included an mRNA degradation element (AU-rich element (ARE) or T3H47 ribozyme; FIG. 13A) produced as part of the transcript produced from the vectors in their native or concatenated state, but that was removed upon mini-circle formation. The mRNA degradation elements destabilize mRNA containing them (FIG. 13B). Experiments were undertaken to demonstrate that the vectors containing the mRNA degradation elements provided for transgene expression from recombinant adeno-associated virus particles (rAAVs) that was dependent on mini-circle formation. A screening vector was prepared containing an mScarlet reporter gene under the control of an enhancer (FIG. 14A) where mini-circle formation would remove an mRNA degradation element from the mScarlet gene transcript. The screening vector was introduced into cells alone or concurrently with a vector encoding FLPo (a codon-optimized flippase gene that encodes a fusion between the SV40 nuclear localization signal and a thermostable version of the Saccharomyces cerevisiae site-specific recombinase FLP). mScarlet fluorescence was measured 3 days post-transduction by flow cytometry (FIG. 14B). FLP expression increased mScarlet mRNA by >100×in vitro.

The above Examples demonstrate that, together, a high-throughput enhancer screening system facilitated by the screening vectors, which leveraged quantitative diverse barcoding, Cre-based specificity scoring, and crosstalk mitigation, represents a broadly useful and scalable technology for gene regulatory element discovery.

### Example 4: Screening a Pooled Library Containing 400 AAV Enhancers

An experiment was undertaken to screen a pooled library of ˜400 enhancers to identify enhancers specific to different cortical interneurons. The library contained 382 novel enhancers, as well as 12 characterized reference regulatory elements (CAG, hSyn, CamKII, mDLX, GRE44, eGHT_017h, eGHT_064h, GfABC1D, S5E2, S5E6, enhancer-less mini-promoter only, enhancer and promoter-less reporter gene only). Each enhancer was assembled with the corresponding barcode pool individually by PCR, pooled, and then assembled into the AAV vector backbone. The assembled DNA library was packaged with PHP.eB and intravenously injected into ACTB-FLP mice and the offsprings of ACTB-FLP mice crossed to a panel of mouse lines expressing Cre in specific interneurons (PV-Cre, SST-Cre, VIP-Cre, Vglut1-Cre). RNA was extracted from the neocortex 3 weeks post injection and converted to cDNA. The barcode-floxed spacer region was PCR amplified from cDNA and sequenced by NGS. Enhancers were identified with different specificities for specific cell types (e.g., neuron subtypes) by assessing: (1) the bulk spacer inversion rate for each enhancer, the relative inversion rate between Cre transgenic lines (e.g., the inversion rate in PV-Cre vs SST-Cre or Vglut1-Cre), (2) the mean enhancer expression strength, (3) the distribution of reads per unique barcodes associated with inverted or non-inverted spacer. The screening vectors facilitated 1) an unbiased test of a subgroup of enhancers and 2) provided an orthogonal measure of whether the expression of the enhancers is adversely affected in pool testing.

The following materials and methods were employed in the above examples.

### Animals

All procedures were performed as approved by the Broad Institute IACUC (0213-06-18 and 0156-03-17-1). C57BL/6J (strain #:000664), ACTB-FLP (B6.Cg-Tg(ACTFLPe)9205Dym/J, strain #:005703), PV-Cre(B6.129P2-Pvalb^{tm1(cre)Arbr}/J, strain #:017320), SST-Cre(STOCK Sst^{tm1(cre)Zjh}/J, strain #:013044), VIP-Cre(STOCK Vip^{tm1(cre)Zjh}/J, strain #:010908), and Vglut1-Cre (B6;129S-Slc17a7^{tm1.1(cre)Hze}/J, strain #: 023527) were obtained from the Jackson Laboratory (JAX). Female ACTB-FLP (homozygous) were crossed with male PV-Cre (homozygous), SST-Cre (homozygous), VIP-Cre (homozygous), or Vglut1-Cre (hemizygous) to yield FLP::PV-Cre, FLP::SST-Cre, FLP::VIP-Cre, or FLP:Vglut1-Cre offsprings, respectively, for enhancer library screening.

### Plasmids

pAAV-EF1a-Cre was from Addgene (#55636). Plasmids constructed were built into an AAV2 genome backbone (pAAV-CAG-NLS-GFP; Addgene #104061). DNA fragments were PCR amplified or synthesized (GenScript or IDT) and cloned into the vector backbone.

### Virus Production

Recombinant AAVs were produced by triple transfection of HEK 293T/17 cells using polyethylenimine (PEI), harvested from the cells and the media 3 days post-transfection, and purified by ultracentrifugation over iodixanol gradients as described in Challis, et al., “Systemic AAV vectors for widespread and targeted gene delivery in rodent,” Nature Protocols, 14:379-414 (2019). For evaluating AAV vectors in HEK cells, AAV vectors in clarified crude lysates were used.

### AAV Titering

To determine AAV titers, 5 μL of each purified virus library was incubated with 100 μL of an endonuclease cocktail consisting of 1000U/mL Turbonuclease (Sigma T4330-50KU) with 1× DNase I reaction buffer (NEB B0303S) in UltraPure DNase/RNase-Free distilled water at 37° C. for one hour. Next, the endonuclease solution was inactivated by adding 5 μL of 0.5M EDTA, pH 8.0 (ThermoFisher Scientific, 15575020) and incubating at room temperature for 5 minutes and then at 70° C. for 10 minutes. To release the encapsidated AAV genomes, 120 μL of a Proteinase K cocktail consisting of 1M NaCl, 1% N-lauroylsarcosine, 100 μg/mL Proteinase K (Qiagen, 19131) in UltraPure DNase/RNase-Free distilled water was added to the mixture and incubated at 56° C. for 2 to 16 hours. The Proteinase K-treated samples were then heat-inactivated at 95° C. for 10 minutes. The released AAV genomes were serial diluted between 460-460,000×in dilution buffer consisting of 1×PCR Buffer (ThermoScientific, N8080129), 2 μg/mL sheared salmon sperm DNA (ThermoScientific, AM9680), and 0.05% Pluronic F68 (ThermoScientific, 24040032) in UltraPure Water (ThermoScientific). 2 μL of the diluted samples were used as input in a ddPCR supermix (Bio-Rad, 1863023). Primers and probes, targeting the ITR or WPRE region, were used for titration, at a final concentration of 900 nM and 250 nM, respectively (Table 1). Droplets were generated using a QX100 Droplet Generator (Bio-Rad) following the manufacturer's protocol. The droplets were transferred to thermocycler and cycled according to the manufacturer's protocol with an annealing/extension of 58° C. for one minute. Finally, droplets were read on a QX100 Droplet Digital System (Bio-Rad) to determine titers.

### Evaluating Expression and Recombination in HEK Cells

HEK 293T/17 cells were cultured in Dulbecco's Modified Eagle Medium with high glucose, sodium pyruvate, GlutaMAX, and Phenol Red (DMEM, Gibco 10569044) supplemented with 5% Fetal Bovine Serum (FBS, Gibco 16000044) and 1×MEM Non-Essential Amino Acids Solution (NEAA, Gibco 11140076). For plasmid transfection, cells were seeded in 12-well or 6-well plates, and transfected with plasmid DNA using Lipofectamine3000 (Thermo Scientific) according to manufacturer's instructions. For virus transduction, cells were grown in 12-well plates with low serum media (DMEM, 2% FBS, 1×NEAA). AAV vectors in crude lysates or purified AAVs were added to cells ˜1 day post seeding. Virus genome/cell was calculated using the number of cells seeded on the plate. Transgene expression was analyzed 2-3 days post transfection or transduction by RT-qPCR or FACS.

Mitigating Enhancer Crosstalk/Interference with FLPout System

Vectors p16 and p38 and screening vectors (p44, p46) were packaged with PHP.eB and intravenously injected into mice via the retro-orbital sinus at 3E11 vg per construct per animal. PHPeB:p16 and PHPeB:p38 were injected into C57BL/6J separately or together to assess level of crosstalk between DLX and PCP2. PHPeB:p44 and PHPeB:p46 were injected into ACTB-FLP mice separately or together to assess mitigation of enhancer crosstalk by the screening vectors. PHPeB:p44 and PHPeB:p46 were also injected separately into C57BL/6J to assess level of spontaneous mini-circle formation in the absence of FLP.

5 weeks post injection, brains were harvested for assessing reporter expression in the cerebrum vs. in the cerebellum by both RT-qPCR and native fluorescence. The two hemispheres were first cut apart along the midline. One hemisphere was directly fixed in 4% PFA-DPBS for 2-3 days at 4° C. and sectioned sagittally using a vibratome. Sections were imaged with a Keyence BZ microscope.

The other hemisphere was used for RNA extraction and RT-qPCR analysis. The cerebellum and the cerebrum tissues were collected into separate tubes. To collect the cerebrum tissue, the thalamus, cerebellum, and brain stem were removed using Graefe forceps (Roboz RS-5136), leaving the cortex, hippocampus, and striatum. These remaining cerebrum tissues were cut into two halves horizontally and collected separately. The mScarlet Cq values in the dorsal half were <1 cycle lower than that in the ventral half (C57BL/6J+p38 or C57BL/6J+p38+p16). Thus the dorsal half of the cerebrum of all mice was used to assess reporter gene expression in the cerebrum.

Cre Based On- and Off-Target Assay Validation with DLX and E2 Enhancers

Construct p36 and p38, containing E2 and DLX enhancers, respectively, were used for Cre inversion assay validation. The constructs contained the features shown in FIG. 1A (i.e., AAV2-ITR-DLX-reporter-BC-Cre-invertable element-WPRE-pA-ITR and AAV2-ITR-PV(E2)-reporter-BC-Cre-invertable element-WPRE-pA-ITR). To model an enhancer screening library, each construct was barcoded with five (S|W)₁₅VHDB barcodes. Oligos containing the (S|W)₁₅VHDB barcodes flanked by sequences overlapping the insertion site on the vector backbone were synthesized by IDT. p36 or p38 was digested with HindIII and EcoRV (New England Biolabs), purified with AmpureXP beads (Beckman Coulter), and assembled with the pooled SW oligos with NEBuilder HiFi DNA Assembly Master Mix (New England Biolabs). Linear DNA was removed with Quick CIP and T5 Exonuclease (New England Biolabs). The final assembled DNA (purified with AmpureXP beads or unpurified) was used directly for transfection to produce AAV vectors using the AAV-PHP.eB capsid.

PHPeB:barcoded-p36 or PHPeB:barcoded-p38 was intravenously injected into C57BL/6J, PV-Cre, SST-Cre, and Vglut1-Cre via the retro-orbital sinus at 3E11 vg per animal (2E11 vg per animal for C57BL/6J injected with PHPeB:barcoded-p38). 5 weeks post injection, brains were harvested and sectioned coronally from the rostral part of the striatum to the rostral edge of the pons in a pre-chilled brain matrix (Zivic Instruments, Inc.) with the ventral side facing up. Isocortex was dissected from each section on a chilled metal plate for total RNA extraction and NGS analysis.

### RNA Extraction and RT-qPCR

RNA from cultured cells (in vitro assays) was extracted using RNeasy Mini Kit (Qiagen) with on-column DNase digestion according to manufacturer's instructions. Mouse tissue (in vivo assay) RNA was extracted using TRIzol Reagent (Invitrogen) and further cleaned up using RNeasy Mini Kit (Qiagen) with on-column DNase digestion, both according to manufacturer's instructions.

cDNA was synthesized using Maxima H Minus Reverse Transcriptase (Thermo Scientific) according to manufacturer's instructions. For in vitro assays, 1-5 pg RNA was converted to cDNA primed by (dT)₂₀ (SEQ ID NO: 44) in 20 μl reactions. For in vivo assays, 5 pg RNA was converted to cDNA primed by (dT)₂₀NV (SEQ ID NO: 45) in 20 μl reactions.

PCR reactions were composed of 1× LightCycler 480 SYBR Green I Master (Roche), 0.5 μM forward primer, 0.5 μM reverse primer, and 1:20 (final) diluted cDNA in a total volume of 20 μl. Real-time qPCR was performed in a C1000 Touch Thermal Cycler (BioRad) with a CFX96 Real-Time System (BioRad) using the following run protocol: 95° C. 5 min, 40 cycles of 95° C. 10 s, 60° C. 10 s, and 72° C. 10 s (with plate read), 95° C. 10 s, 65° C. 1 min, 65° C. to 95° C., increment 0.5° C. per 5 s with plate read. The quantification cycle Cq was determined by BioRad CFX Maestro software using the default settings (baseline subtracted curve fit and single threshold). Mean Cq was used for calculating fold change. 2\hat{ }ΔCq

### Next-Generation Sequencing (NGS) Sample Preparation

Tissue RNA was extracted and 5 μg of RNA per animal was converted to cDNA as described above. A 364 bp region on the viral cDNA containing the barcode and the floxed DNA was enriched and attached with sample indexes and Illumina sequencing adapters by two rounds of PCR using Q5 Hot Start High-Fidelity 2×Master Mix (New England Biolabs).

For PCR round 1, 8 PCR reactions per cDNA sample were performed, each using one of 8 sets of forward and reverse primers (see Table 3). Each primer set contained Illumina read1 or read2 sequences, 0-8 N's, and the binding sites on the viral cDNA. Each PCR reaction was composed of 1× master mix, 0.5 μM forward primer, 0.5 μM reverse primer, and 1:20 (final) diluted cDNA in a total volume of 25 μl. PCR run protocol was as follows: 98° C. 30 s; variable cycles of 98° C. 10 s, 6TC 30 s, and 72° C. 1 min 30 s; 72° C. 5 min. PCR cycle number was determined by qPCR using the same condition except additional Sybr Green in the PCR reaction. PCR products were pooled per cDNA sample and purified with Ampure XP beads (Beckman Coulter) according to the manufacturer's instructions.

PCR round 1 products were attached on Illumina adaptors and dual indexes in PCR round2 using NEBNext Multiplex Oligos for Illumina (New England Biolabs E7600S). Each PCR reaction was composed of 1× master mix, 0.5 μM i5 primer, 0.5 μM i7 primer, and 1:10 (final) PCR round1 product in a total volume of 25 μl. PCR run protocol was as follows: 98° C. 30 s; 7 cycles of 98° C. 10 s, 72° C. 20 s, and 72° C. 2 min; 72° C. 5 min. PCR products were purified with Ampure XP beads (Beckman Coulter) according to the manufacturer's instructions.

To quantify the amount of second round PCR product for NGS an Agilent High Sensitivity DNA Kit (Agilent, 5067-4626) was used with an Agilent 2100 Bioanalyzer system. Second round PCR products were then pooled and diluted to 2-4 nM in 10 mM Tris-HCl, pH 8.5 and sequenced on an Illumina NextSeq 550 following the manufacturer's instructions using a NextSeq 500/550 Mid or High Output Kit (Illumina, 20024904 or 20024907), or on an Illumina NextSeq 1000 following the manufacturer's instructions using NextSeq P2 v3 kits (Illumina, 20046812). Reads were allocated as follows: I1: 8, I2: 8, R1: 150, R2: 0.

TABLE 3
Next-Generation Sequencing (NGS) PCR1 primers

| | |
|---|---|
| NGS PCR1 primer | CTTTCCCTACACGACGCTCTTCCGATCTNNNNNNNNGGCATGGACGAGCT |
| set 1 | GTATAAGTA (SEQ ID NO: 46) |
| NGS PCR1 primer | GGAGTTCAGACGTGTGCTCTTCCGATCTAAGCAGCGTATCCACATAGCG |
| set 1 | (SEQ ID NO: 47) |
| NGS PCR1 primer | CTTTCCCTACACGACGCTCTTCCGATCTNNNNNNNGGCATGGACGAGCTG |
| set 2 | TATAAGTA (SEQ ID NO: 48) |
| NGS PCR1 primer | GGAGTTCAGACGTGTGCTCTTCCGATCTNAAGCAGCGTATCCACATAGCG |
| set 2 | (SEQ ID NO: 49) |
| NGS PCR1 primer | CTTTCCCTACACGACGCTCTTCCGATCTNNNNNNGGCATGGACGAGCTGT |
| set 3 | ATAAGTA (SEQ ID NO: 50) |
| NGS PCR1 primer | GGAGTTCAGACGTGTGCTCTTCCGATCTNNAAGCAGCGTATCCACATAGC |
| set 3 | G (SEQ ID NO: 51) |
| NGS PCR1 primer | CTTTCCCTACACGACGCTCTTCCGATCTNNNNNGGCATGGACGAGCTGTA |
| set 4 | TAAGTA (SEQ ID NO: 52) |
| NGS PCR1 primer | GGAGTTCAGACGTGTGCTCTTCCGATCTNNNAAGCAGCGTATCCACATAG |
| set 4 | CG (SEQ ID NO: 53) |
| NGS PCR1 primer | CTTTCCCTACACGACGCTCTTCCGATCTNNNNGGCATGGACGAGCTGTAT |
| set 5 | AAGTA (SEQ ID NO: 54) |
| NGS PCR1 primer | GGAGTTCAGACGTGTGCTCTTCCGATCTNNNNAAGCAGCGTATCCACATA |
| set 5 | GCG (SEQ ID NO: 55) |
| NGS PCR1 primer | CTTTCCCTACACGACGCTCTTCCGATCTNNNGGCATGGACGAGCTGTATA |
| set 6 | AGTA (SEQ ID NO: 56) |
| NGS PCR1 primer | GGAGTTCAGACGTGTGCTCTTCCGATCTNNNNNAAGCAGCGTATCCACAT |
| set 6 | AGCG (SEQ ID NO: 57) |
| NGS PCR1 primer | CTTTCCCTACACGACGCTCTTCCGATCTNNGGCATGGACGAGCTGTATAA |
| set 7 | GTA (SEQ ID NO: 58) |
| NGS PCR1 primer | GGAGTTCAGACGTGTGCTCTTCCGATCTNNNNNNAAGCAGCGTATCCACA |
| set 7 | TAGCG (SEQ ID NO: 59) |
| NGS PCR1 primer | CTTTCCCTACACGACGCTCTTCCGATCTNGGCATGGACGAGCTGTATAAG |
| set 8 | TA (SEQ ID NO: 60) |
| NGS PCR1 primer | GGAGTTCAGACGTGTGCTCTTCCGATCTNNNNNNNAAGCAGCGTATCCAC |
| set 8 | ATAGCG (SEQ ID NO: 61) |

### Next-Generation Sequencing (NGS) Data Processing

Sequencing data was demultiplexed with bcl2fastq (version v2.20.0.422) using default parameters. The sequence reads (excluding Illumina barcodes) were aligned to a short reference multifasta file of the Forward (corresponding to an uninverted spacer sequence) and Inverted (corresponding to an inverted spacer sequence) sequences:

>Forward
(SEQ ID NO: 62)
agtacgaacgctccgagggccgccactccaccggcggcatggacgagctgtaTaagtaaGATATCNNNNNNNNNNNN
NNNNNNNAAGCTTctgcgttgttgatattgtggacctcgGAATTCAattaTTCGTATAGCATACATTAT
ACGAAGTTATGTAGACAATCCTTTGGTCCGAAGTATGTACAACATTTGCGGCCTAAA
GACAAACCGCTCCATGGTGAAAACGACTAAGGGTACCCAGGAGAATATGAGCTATA
AaTTgcTATAATGTATGCTATACGAAGTTATgaattcatcgataatcaacctctggattacaaaatttgtgaaagatt
gactggtattcttaactatgttgctccttttacgctatgtggatacgctgctttaatgcctttgtatcatgctattgcttcccgtatggc
tttcatttt
>Inverted
(SEQ ID NO: 63)
agtacgaacgctccgagggccgccactccaccggcggcatggacgagctgtaTaagtaaGATATCNNNNNNNNNNNN
NNNNNNNAAGCTTctgcgttgttgatattgtggacctcgGAATTCAattaTTCGTATAGCATACATTAT
AgcAAtTTATAGCTCATATTCTCCTGGGTACCCTTAGTCGTTTTCACCATGGAGCGGTT
TGTCTTTAGGCCGCAAATGTTGTACATACTTCGGACCAAAGGATTGTCTACATAACT
TCGTATAATGTATGCTATACGAAGTTATgaattcatcgataatcaacctctggattacaaaatttgtgaaagattga
ctggtattcttaactatgttgctccttttacgctatgtggatacgctgctttaatgcctttgtatcatgctattgcttcccgtatggctt
tcatttt

Alignment was performed with bowtie2 (version 2.4.1) (Langmead and Salzberg 2012) with the following parameters: --end-to-end --very-sensitive --np 0---n-ceil L,21,0.5 --xeq-N 1 --reorder --score-min L,−0.6, −0.6, −5 8 −3 8. Resulting sam files from bowtie2 were sorted by read and compressed to bam files with samtools (version 1.11-2-g26d7c73, htslib version 1.11-9-g2264113) (Danecek P, Bonfield J K, Liddle J, et al. Twelve years of SAMtools and BCFtools. Gigascience. 2021; 10(2):giab008. doi:10.1093/gigascience/giab008; and Li H, Handsaker B, Wysoker A, et al, “The Sequence Alignment/Map format and SAMtools,” Bioinformatics. 2009; 25:2078-9).

Python (version 3.8.3) scripts and pysam (version 0.15.4) were used to flexibly extract the 19 nucleotide barcode sequences from each amplicon read. Each read was assigned to one of the following bins: Failed, Invalid, or Valid. Failed reads were defined as reads that did not align to the reference sequence, or that had an insertion or deletion in the insertion region (e.g., 18 bases instead of 19 bases). Invalid reads were defined as reads whose 19 bases were successfully extracted, but matched any of the following conditions: 1) Any one base of the 19 bases had a quality score (AKA Phred score, QScore) below 20, i.e., error probability >1/100, 2) Any one base was undetermined, i.e., “N”. Valid reads were defined as reads that did not fit into either the Failed or Invalid bins. The Failed and Invalid reads were collected and analyzed for quality control purposes, and all subsequent analyses were performed on the Valid reads.

Count data for valid reads was aggregated per sequence, per sample, and was stored in a pivot table format, with barcode nucleotide sequences on the rows, and samples (Illumina sample indexes) on the columns. Barcode sequences not detected in samples were assigned a count of 0. The first 15 nucleotides of the barcode sequences were converted to S|W sequences. Barcode sequences with the total read counts <5 across all samples sequenced in the same next-generation sequencing (NGS) run, or with the corresponding S|W sequences not present in the starting pool were removed from further calculation. Total read counts per sample were calculated by taking the sum of all read counts of the sample. Total number of unique barcodes were counted considering inverted and forward as different barcodes.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A vector comprising a polynucleotide comprising a cis regulatory element and a promoter sequence, each within a region of the polynucleotide defined by two recombinase recognition sites, wherein contacting the polynucleotide with a recombinase forms a mini-circle and stabilizes mRNA transcribed from the polynucleotide in a cell.

2. The vector of claim 1, wherein the polynucleotide comprises a viral genome.

3. The vector of claim 1, wherein the two recombinase recognition sites are flippase recognition target (FRT) sites.

4. The vector of claim 1, wherein the polynucleotide further comprises a 3′ untranslated region (UTR) within the region defined by the two recombinase recognition sites and 5′ of the cis regulatory sequence and promoter.

5. The vector of claim 4, wherein mini-circle formation positions the 3′ UTR 3′ of the cis regulatory element and promoter sequence so that the mRNA includes the 3′ UTR.

6. The vector of claim 4, wherein the 3′ UTR stabilizes the mRNA transcribed from the polynucleotide in the cell.

7. The vector of claim 1, wherein the polynucleotide further comprises an mRNA destabilizing element outside of the region defined by the two recombinase recognition sites and 3′ of the cis regulatory element and promoter sequence.

8. The vector of claim 7, wherein the mRNA destabilizing element destabilizes the mRNA prior to mini-circle formation.

9. The vector of claim 1, wherein the vector comprises a barcode comprising the sequence (S/W)₁₅VHDB.

10. The vector of claim 1, wherein the polynucleotide further comprises an invertible spacer sequence transcribed under the control of the promoter, wherein the invertible spacer sequence is disposed between a second set of recombinase recognition sites, which are within the region defined by the region defined within the first two recombinase recognition sites.

11. An isolated recombinant adeno-associated virus (rAAV) particle comprising the polynucleotide of claim 1.

12. A polynucleotide library of cis regulatory sequences, wherein the cis regulatory sequences in the library are each encoded by a vector of claim 1.

13. A composition comprising the vector of claim 1.

14. A method for screening cis regulatory elements for cell type-specific expression, the method comprising: administering to a subject a vector comprising a polynucleotide comprising the following disposed within a region of the polynucleotide defined by two recombinase recognition sites: a cis regulatory element, a promoter sequence, a barcode, and an invertible spacer sequence that is disposed within a second set of recombinase recognition sites.

15. The method of claim 14, wherein contacting the polynucleotide with a recombinase forms a mini-circle and stabilizes mRNA transcribed from the polynucleotide in a cell.

16. The method of claim 14, wherein each cis regulatory element corresponds to a unique readable sequence.

17. The method of claim 14, wherein the method is associated with a reduction in cross-talk between the cis regulatory sequences relative to a method using vectors that do not contain the first set of two recombinase recognition sites.

18. A vector comprising in order from 5′ to 3′: a flippase recognition target (FRT) site, a 3′ UTR, a cis regulatory element, a promoter, a (S/W)₁₅VHDB bar code, a lox site, an invertible spacer sequence, a second lox site, and a second flippase recognition target (FRT) site.

19. A method for screening cis regulatory elements for cell type-specific expression, the method comprising:
a) administering to a rodent one or more vectors of claim 1, wherein the rodent expresses a Cre and a flippase (FLP); and
b) sequencing mRNA from the rodent to detect the barcodes and inversion status of the invertible spacer sequences.

20. A method for screening cis regulatory elements for cell type-specific expression, the method comprising: contacting cells with a vector comprising a polynucleotide comprising the following disposed within a region of the polynucleotide defined by two recombinase recognition sites: a cis regulatory element, a promoter sequence, a barcode, and an invertible spacer sequence that is disposed within a second set of recombinase recognition sites.
